(19) United States
(12) Patent Application Publication
(10) Pub. No.: US 2025/0291382 A1
(43) Pub. Date: Sep. 18, 2025

(54) Nerd of the Rings -- Smart Rings with Optical Sensors for Obtaining Biometric Information

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 19/221,114

(22) Filed: May 28, 2025

Related U.S. Application Data
(63) Continuation-in-part of application No. 19/044,788, filed on Feb. 4, 2025.
Application No. 19/044,788 is a continuation-in-part of application No. 19/008,398, filed on Jan. 2, 2025.
Application No. 19/008,398 is a continuation-in-part of application No. 19/008,344, filed on Jan. 2, 2025.
Application No. 19/008,344 is a continuation-in-part of application No. 19/002,587, filed on Dec. 26, 2024.
Application No. 19/002,587 is a continuation-in-part of application No. 19/002,583, filed on Dec. 26, 2024.
Application No. 19/002,583 is a continuation-in-part of application No. 18/977,825, filed on Dec. 11, 2024.
Application No. 18/977,825 is a continuation-in-part of application No. 18/977,824, filed on Dec. 11, 2024.
Application No. 18/977,824 is a continuation-in-part of application No. 18/885,728, filed on Sep. 15, 2024.
Application No. 18/885,728 is a continuation-in-part of application No. 18/775,128, filed on Jul. 17, 2024.
Application No. 18/977,825 is a continuation-in-part of application No. 18/929,026, filed on Oct. 28, 2024.
Application No. 18/929,026 is a continuation-in-part of application No. 18/885,728, filed on Sep. 15, 2024.
Application No. 18/885,728 is a continuation-in-part of application No. 18/775,128, filed on Jul. 17, 2024.
Application No. 18/775,128 is a continuation-in-part of application No. 18/617,950, filed on Mar. 27, 2024.
Application No. 18/617,950 is a continuation-in-part of application No. 18/121,841, filed on Mar. 15, 2023, now abandoned.
Application No. 18/929,026 is a continuation-in-part of application No. 18/885,728, filed on Sep. 15, 2024.
Application No. 18/885,728 is a continuation-in-part of application No. 18/775,128, filed on Jul. 17, 2024.
Application No. 18/775,128 is a continuation-in-part of application No. 18/121,841, filed on Mar. 15, 2023, now abandoned.
Application No. 18/885,728 is a continuation-in-part of application No. 18/775,128, filed on Jul. 17, 2024.
Application No. 18/775,128 is a continuation-in-part of application No. 18/617,950, filed on Mar. 27, 2024.
Application No. 18/617,950 is a continuation-in-part of application No. 18/121,841, filed on Mar. 15, 2023, now abandoned.
Application No. 18/775,128 is a continuation-in-part of application No. 18/617,950, filed on Mar. 27, 2024.
Application No. 18/617,950 is a continuation-in-part of application No. 18/121,841, filed on Mar. 15, 2023, now abandoned.
Application No. 18/121,841 is a continuation-in-part of application No. 18/121,841, filed on Mar. 15, 2023, now abandoned.
Application No. 18/121,841 is a continuation-in-part of application No. 17/903,746, filed on Sep. 6, 2022, now abandoned.
Application No. 17/903,746 is a continuation-in-part of application No. 17/239,960, filed on Apr. 26, 2021, now abandoned.
Application No. 17/239,960 is a continuation-in-part of application No. 16/737,052, filed on Jan. 8, 2020, now Pat. No. 11,754,542.
Application No. 16/737,052 is a continuation-in-part of application No. 17/239,960, filed on Apr. 26, 2021, now abandoned.
Application No. 17/239,960 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/294,746, filed on Oct. 16, 2016, now Pat. No. 10,627,861.
Application No. 15/294,746 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/944,746, filed on Apr. 3, 2018, now abandoned.
Application No. 15/963,061 is a continuation-in-part of application No. 14/992,073, filed on Jan. 11, 2016, now abandoned.
Application No. 14/992,073 is a continuation-in-part of application No. 14/550,953, filed on Nov. 22, 2014, now abandoned.
Application No. 14/550,953 is a continuation-in-part of application No. 15/431,769, filed on Feb. 14, 2017, now abandoned.
Application No. 15/431,769 is a continuation-in-part of application No. 14/951,475, filed on Nov. 24, 2015, now Pat. No. 10,314,492.
Application No. 14/951,475 is a continuation-in-part of application No. 15/294,746, filed on Oct. 16, 2016, now Pat. No. 10,627,861.
Application No. 15/294,746 is a continuation-in-part of application No. 15/206,215, filed on Jul. 8, 2016, now abandoned.
Application No. 15/206,215 is a continuation-in-part of application No. 14/992,073, filed on Jan. 11, 2016, now abandoned.
Application No. 14/992,073 is a continuation-in-part of application No. 14/330,649, filed on Jul. 14, 2014, now abandoned.
Application No. 14/330,649 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/948,308, filed on Nov. 21, 2015, now abandoned.
Application No. 14/992,073 is a continuation-in-part of application No. 14/562,719, filed on Dec. 7, 2014, now Pat. No. 10,130,277.
Application No. 14/562,719 is a continuation-in-part of application No. 13/616,238, filed on Sep. 14, 2012, now abandoned.
Application No. 14/951,475 is a continuation-in-part of application No. 14/071,112, filed on Nov. 4, 2013, now abandoned.
Application No. 14/071,112 is a continuation-in-part of application No. 13/901,131, filed on May 23, 2013, now Pat. No. 9,536,449.
Application No. 14/948,308 is a continuation-in-part of application No. 14/550,953, filed on Nov. 22, 2014, now abandoned.
Application No. 14/550,953 is a continuation-in-part of application No. 14/449,387, filed on Aug. 1, 2014, now abandoned.
Application No. 14/449,387 is a continuation-in-part of application No. 14/132,292, filed on Dec. 18, 2013, now Pat. No. 9,442,100.
Application No. 14/132,292 is a continuation-in-part of application No. 13/901,099, filed on May 23, 2013, now Pat. No. 9,254,099.
Application No. 14/330,649 is a continuation-in-part of application No. 13/523,739, filed on Jun. 14, 2012, now Pat. No. 9,042,596.
(60) Provisional application No. 61/932,517, filed on Jan. 28, 2014.
(60) Provisional application No. 62/245,311, filed on Oct. 23, 2015.
(60) Provisional application No. 62/311,462, filed on Mar. 22, 2016.
(60) Provisional application No. 62/349,277, filed on Jun. 13, 2016.
(60) Provisional application No. 62/349,277, filed on Jun. 13, 2016.
(60) Provisional application No. 62/349,277, filed on Jun. 13, 2016.
(60) Provisional application No. 62/439,147, filed on Dec. 26, 2016.
(60) Provisional application No. 62/439,147, filed on Dec. 26, 2016.
(60) Provisional application No. 62/549,587, filed on Aug. 24, 2017.
(60) Provisional application No. 62/800,478, filed on Feb. 2, 2019.
(60) Provisional application No. 62/814,713, filed on Mar. 6, 2019.
(60) Provisional application No. 62/814,692, filed on Mar. 6, 2019.
(60) Provisional application No. 62/814,713, filed on Mar. 6, 2019.
(60) Provisional application No. 62/814,692, filed on Mar. 6, 2019.
(60) Provisional application No. 62/857,942, filed on Jun. 6, 2019.
(60) Provisional application No. 62/857,942, filed on Jun. 6, 2019.
(60) Provisional application No. 62/930,013, filed on Nov. 4, 2019.
(60) Provisional application No. 63/171,838, filed on Apr. 7, 2021.
(60) Provisional application No. 63/279,773, filed on Nov. 16, 2021.
(60) Provisional application No. 63/542,077, filed on Oct. 2, 2023.
(60) Provisional application No. 63/542,077, filed on Oct. 2, 2023.

(51) Int. Cl. G06F 1/16; G02B 26/08; G02B 26/10; G06F 3/01

(52) U.S. Cl. CPC G06F 1/163; G02B 26/085; G02B 26/101; G06F 1/1684; G06F 3/015

(57) ABSTRACT

Disclosed herein are smart finger rings with light emitters and light receivers which collect biometric information concerning a person's health. Light from light emitters is received by light receivers after the light has been reflected by and/or transmitted through the person's finger. Several designs disclosed herein address the problem of size variation via innovative compressible, expandable, and/or adjustable-size components. Several designs disclosed herein address the problem of unintentional ring rotation via innovative movable components which change the circumferential locations and/or angles at which light beams from light emitters exit the ring toward a person's finger.

### CROSS-REFERENCE TO RELATED APPLICATIONS

This application is: a continuation in part of U.S. patent application Ser. No. 19/044,788 filed on 2025 Feb. 4; and a continuation in part of U.S. patent application Ser. No. 19/008,398 filed on 2025 Jan. 2.

U.S. patent application Ser. No. 19/044,788 was: a continuation in part of U.S. patent application Ser. No. 19/008,398 filed on 2025 Jan. 2; a continuation in part of U.S. patent application Ser. No. 19/008,344 filed on 2025 Jan. 2; a continuation in part of U.S. patent application Ser. No. 19/002,587 filed on 2024 Dec. 26; a continuation in part of U.S. patent application Ser. No. 19/002,583 filed on 2024 Dec. 26; a continuation in part of U.S. patent application Ser. No. 18/977,825 filed on 2024 Dec. 11; a continuation in part of U.S. patent application Ser. No. 18/977,824 filed on 2024 Dec. 11; a continuation in part of U.S. patent application Ser. No. 18/885,728 filed on 2024 Sep. 15; and a continuation in part of U.S. patent application Ser. No. 18/775,128 filed on 2024 Jul. 17.

U.S. patent application Ser. No. 19/008,398 was a continuation-in-part of U.S. patent application Ser. No. 19/008,344 filed on 2025 Jan. 2. U.S. patent application Ser. No. 19/008,398 was a continuation-in-part of U.S. patent application Ser. No. 19/002,587 filed on 2024 Dec. 26. U.S. patent application Ser. No. 19/008,398 was a continuation-in-part of U.S. patent application Ser. No. 19/002,583 filed on 2024 Dec. 26. U.S. patent application Ser. No. 19/008,398 was a continuation-in-part of U.S. patent application Ser. No. 18/977,825 filed on 2024 Dec. 11.

U.S. patent application Ser. No. 19/008,344 was a continuation-in-part of U.S. patent application Ser. No. 19/002,587 filed on 2024 Dec. 26. U.S. patent application Ser. No. 19/008,344 was a continuation-in-part of U.S. patent application Ser. No. 19/002,583 filed on 2024 Dec. 26. U.S. patent application Ser. No. 19/008,344 was a continuation-in-part of U.S. patent application Ser. No. 18/977,825 filed on 2024 Dec. 11. U.S. patent application Ser. No. 19/008,344 was a continuation-in-part of U.S. patent application Ser. No. 18/977,824 filed on 2024 Dec. 11.

U.S. patent application Ser. No. 18/977,825 was a continuation-in-part of U.S. patent application Ser. No. 18/929,026 filed on 2024 Oct. 28. U.S. patent application Ser. No. 18/977,825 was a continuation-in-part of U.S. patent application Ser. No. 18/885,728 filed on 2024 Sep. 15. U.S. patent application Ser. No. 18/977,825 was a continuation-in-part of U.S. patent application Ser. No. 18/775,128 filed on 2024 Jul. 17. U.S. patent application Ser. No. 18/977,825 was a continuation-in-part of U.S. patent application Ser. No. 18/617,950 filed on 2024 Mar. 27. U.S. patent application Ser. No. 18/977,825 was a continuation-in-part of U.S. patent application Ser. No. 18/121,841 filed on 2023 Mar. 15.

U.S. patent application Ser. No. 18/929,026 was a continuation-in-part of U.S. patent application Ser. No. 18/885,728 filed on 2024 Sep. 15. U.S. patent application Ser. No. 18/929,026 was a continuation-in-part of U.S. patent application Ser. No. 18/775,128 filed on 2024 Jul. 17. U.S. patent application Ser. No. 18/929,026 was a continuation-in-part of U.S. patent application Ser. No. 18/121,841 filed on 2023 Mar. 15. U.S. patent application Ser. No. 18/885,728 was a continuation-in-part of U.S. patent application Ser. No. 18/775,128 filed on 2024 Jul. 17. U.S. patent application Ser. No. 18/885,728 was a continuation-in-part of U.S. patent application Ser. No. 18/617,950 filed on 2024 Mar. 27. U.S. patent application Ser. No. 18/885,728 claimed the priority benefit of U.S. provisional application 63/542,077 filed on 2023 Oct. 2. U.S. patent application Ser. No. 18/885,728 was a continuation-in-part of U.S. patent application Ser. No. 18/121,841 filed on 2023 Mar. 15.

U.S. patent application Ser. No. 18/775,128 was a continuation-in-part of U.S. patent application Ser. No. 18/617,950 filed on 2024 Mar. 27. U.S. patent application Ser. No. 18/775,128 was a continuation-in-part of U.S. patent application Ser. No. 18/121,841 filed on 2023 Mar. 15. U.S. patent application Ser. No. 18/617,950 claimed the priority benefit of U.S. provisional application 63/542,077 filed on 2023 Oct. 2. U.S. patent application Ser. No. 18/617,950 was a continuation-in-part of U.S. patent application Ser. No. 18/121,841 filed on 2023 Mar. 15. U.S. patent application Ser. No. 18/121,841 was a continuation-in-part of U.S. patent application Ser. No. 17/903,746 filed on 2022 Sep. 6. U.S. patent application Ser. No. 18/121,841 was a continuation-in-part of U.S. patent application Ser. No. 17/239,960 filed on 2021 Apr. 26. U.S. patent application Ser. No. 18/121,841 was a continuation-in-part of U.S. patent application Ser. No. 16/737,052 filed on 2020 Jan. 8.

U.S. patent application Ser. No. 17/903,746 was a continuation-in-part of U.S. patent application Ser. No. 16/568,580 filed on 2019 Sep. 12. U.S. patent application Ser. No. 17/903,746 was a continuation-in-part of U.S. patent application Ser. No. 16/737,052 filed on 2020 Jan. 8. U.S. patent application Ser. No. 17/903,746 was a continuation-in-part of U.S. patent application Ser. No. 17/239,960 filed on 2021 Apr. 26. U.S. patent application Ser. No. 17/903,746 claimed the priority benefit of U.S. provisional application 63/279,773 filed on 2021 Nov. 16. U.S. patent application Ser. No. 17/239,960 claimed the priority benefit of U.S. provisional application 63/171,838 filed on 2021 Apr. 7. U.S. patent application Ser. No. 17/239,960 was a continuation-in-part of U.S. patent application Ser. No. 16/737,052 filed on 2020 Jan. 8.

U.S. patent application Ser. No. 16/737,052 claimed the priority benefit of U.S. provisional application 62/930,013 filed on 2019 Nov. 4. U.S. patent application Ser. No. 16/737,052 claimed the priority benefit of U.S. provisional application 62/857,942 filed on 2019 Jun. 6. U.S. patent application Ser. No. 16/737,052 claimed the priority benefit of U.S. provisional application 62/814,713 filed on 2019 Mar. 6. U.S. patent application Ser. No. 16/737,052 claimed the priority benefit of U.S. provisional application 62/814,692 filed on 2019 Mar. 6. U.S. patent application Ser. No. 16/737,052 claimed the priority benefit of U.S. provisional application 62/800,478 filed on 2019 Feb. 2. U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25 which issued as U.S. Pat. No. 10,772,559 on 2020 Sep. 15. U.S. patent application Ser. No. 16/737,052 was a continuation-in-part of U.S. patent application Ser. No. 15/725,330 filed on 2017 Oct. 5 which issued as U.S. Pat. No. 10,607,507 on 2020 Mar. 31. U.S. patent application Ser. No. 16/737,052 was a continuation-in-part of U.S. patent application Ser. No. 15/431,769 filed on 2017 Feb. 14. U.S. patent application Ser. No. 16/737,052 was a continuation-in-part of U.S. patent application Ser. No. 15/294,746 filed on 2016 Oct. 16 which issued as U.S. Pat. No. 10,627,861 on 2020 Apr. 21.

U.S. patent application Ser. No. 16/568,580 claimed the priority benefit of U.S. provisional application 62/857,942 filed on 2019 Jun. 6. U.S. patent application Ser. No. 16/568,580 claimed the priority benefit of U.S. provisional application 62/814,713 filed on 2019 Mar. 6. U.S. patent application Ser. No. 16/568,580 claimed the priority benefit of U.S. provisional application 62/814,692 filed on 2019 Mar. 6. U.S. patent application Ser. No. 16/568,580 was a continuation-in-part of U.S. patent application Ser. No. 15/963,061 filed on 2018 Apr. 25 which issued as U.S. Pat. No. 10,772,559 on 2020 Sep. 15. U.S. patent application Ser. No. 16/568,580 was a continuation-in-part of U.S. patent application Ser. No. 15/725,330 filed on 2017 Oct. 5 which issued as U.S. Pat. No. 10,607,507 on 2020 Mar. 31. U.S. patent application Ser. No. 16/568,580 was a continuation-in-part of U.S. patent application Ser. No. 15/431,769 filed on 2017 Feb. 14. U.S. patent application Ser. No. 16/568,580 was a continuation-in-part of U.S. patent application Ser. No. 15/418,620 filed on 2017 Jan. 27. U.S. patent application Ser. No. 16/568,580 was a continuation-in-part of U.S. patent application Ser. No. 15/294,746 filed on 2016 Oct. 16 which issued as U.S. Pat. No. 10,627,861 on 2020 Apr. 21.

U.S. patent application Ser. No. 15/963,061 was a continuation-in-part of U.S. patent application Ser. No. 14/992,073 filed on 2016 Jan. 11. U.S. patent application Ser. No. 15/963,061 was a continuation-in-part of U.S. patent application Ser. No. 14/550,953 filed on 2014 Nov. 22. U.S. patent application Ser. No. 15/725,330 claimed the priority benefit of U.S. provisional application 62/549,587 filed on 2017 Aug. 24. U.S. patent application Ser. No. 15/725,330 claimed the priority benefit of U.S. provisional application 62/439,147 filed on 2016 Dec. 26. U.S. patent application Ser. No. 15/725,330 was a continuation-in-part of U.S. patent application Ser. No. 15/431,769 filed on 2017 Feb. 14. U.S. patent application Ser. No. 15/725,330 was a continuation-in-part of U.S. patent application Ser. No. 14/951,475 filed on 2015 Nov. 24 which issued as U.S. Pat. No. 10,314,492 on 2019 Jun. 11.

U.S. patent application Ser. No. 15/431,769 claimed the priority benefit of U.S. provisional application 62/439,147 filed on 2016 Dec. 26. U.S. patent application Ser. No. 15/431,769 claimed the priority benefit of U.S. provisional application 62/349,277 filed on 2016 Jun. 13. U.S. patent application Ser. No. 15/431,769 claimed the priority benefit of U.S. provisional application 62/311,462 filed on 2016 Mar. 22. U.S. patent application Ser. No. 15/431,769 was a continuation-in-part of U.S. patent application Ser. No. 15/294,746 filed on 2016 Oct. 16 which issued as U.S. Pat. No. 10,627,861 on 2020 Apr. 21. U.S. patent application Ser. No. 15/431,769 was a continuation-in-part of U.S. patent application Ser. No. 15/206,215 filed on 2016 Jul. 8. U.S. patent application Ser. No. 15/431,769 was a continuation-in-part of U.S. patent application Ser. No. 14/992,073 filed on 2016 Jan. 11. U.S. patent application Ser. No. 15/431,769 was a continuation-in-part of U.S. patent application Ser. No. 14/330,649 filed on 2014 Jul. 14.

U.S. patent application Ser. No. 15/418,620 claimed the priority benefit of U.S. provisional application 62/297,827 filed on 2016 Feb. 20. U.S. patent application Ser. No. 15/418,620 was a continuation-in-part of U.S. patent application Ser. No. 14/951,475 filed on 2015 Nov. 24 which issued as U.S. Pat. No. 10,314,492 on 2019 Jun. 11. U.S. patent application Ser. No. 15/294,746 claimed the priority benefit of U.S. provisional application 62/349,277 filed on 2016 Jun. 13. U.S. patent application Ser. No. 15/294,746 claimed the priority benefit of U.S. provisional application 62/245,311 filed on 2015 Oct. 23. U.S. patent application Ser. No. 15/294,746 was a continuation-in-part of U.S. patent application Ser. No. 14/951,475 filed on 2015 Nov. 24 which issued as U.S. Pat. No. 10,314,492 on 2019 Jun. 11.

U.S. patent application Ser. No. 15/206,215 claimed the priority benefit of U.S. provisional application 62/349,277 filed on 2016 Jun. 13. U.S. patent application Ser. No. 15/206,215 was a continuation-in-part of U.S. patent application Ser. No. 14/951,475 filed on 2015 Nov. 24 which issued as U.S. Pat. No. 10,314,492 on 2019 Jun. 11. U.S. patent application Ser. No. 15/206,215 was a continuation-in-part of U.S. patent application Ser. No. 14/948,308 filed on 2015 Nov. 21. U.S. patent application Ser. No. 14/992,073 was a continuation-in-part of U.S. patent application Ser. No. 14/562,719 filed on 2014 Dec. 7 which issued as U.S. Pat. No. 10,130,277 on 2018 Nov. 20. U.S. patent application Ser. No. 14/992,073 was a continuation-in-part of U.S. patent application Ser. No. 13/616,238 filed on 2012 Sep. 14.

U.S. patent application Ser. No. 14/951,475 was a continuation-in-part of U.S. patent application Ser. No. 14/071,112 filed on 2013 Nov. 4. U.S. patent application Ser. No. 14/951,475 was a continuation-in-part of U.S. patent application Ser. No. 13/901,131 filed on 2013 May 23 which issued as U.S. Pat. No. 9,536,449 on 2017 Jan. 3. U.S. patent application Ser. No. 14/948,308 was a continuation-in-part of U.S. patent application Ser. No. 14/550,953 filed on 2014 Nov. 22. U.S. patent application Ser. No. 14/948,308 was a continuation-in-part of U.S. patent application Ser. No. 14/449,387 filed on 2014 Aug. 1. U.S. patent application Ser. No. 14/948,308 was a continuation-in-part of U.S. patent application Ser. No. 14/132,292 filed on 2013 Dec. 18 which issued as U.S. Pat. No. 9,442,100 on 2016 Sep. 13. U.S. patent application Ser. No. 14/948,308 was a continuation-in-part of U.S. patent application Ser. No. 13/901,099 filed on 2013 May 23 which issued as U.S. Pat. No. 9,254,099 on 2016 Feb. 9. U.S. patent application Ser. No. 14/562,719 claimed the priority benefit of U.S. provisional application 61/932,517 filed on 2014 Jan. 28. U.S. patent application Ser. No. 14/330,649 was a continuation-in-part of U.S. patent application Ser. No. 13/523,739 filed on 2012 Jun. 14 which issued as U.S. Pat. No. 9,042,596 on 2015 May 26.

The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to smart rings for measuring biometric parameters.

### INTRODUCTION

Smart finger rings with optical sensors have advantages over mobile handheld devices (such as cellphones) and non-mobile devices (such as stationary medical equipment) for monitoring a person's biometric parameters: to diagnosis adverse health conditions; to provide an alert in case of an adverse health event; to provide a feedback and/or control loop for the operation of implanted medical devices; and to help people maintain their health and prevent illness. Due to their consistent proximity to a person's body, low-profile, and social acceptability, smart finger rings can monitor biometric parameters more broadly and consistently than handheld devices.

There are also challenges in the development of smart finger rings. For example, the circumferences of fingers can change over time due to water retention or loss, weight gain or loss, or other factors, which can create gaps between optical sensors and the finger which cause measurement errors. Also, rings can unintentionally rotate around a person's finger, shifting optical sensors away from preferred measurement locations.

### Review of the Relevant Art

U.S. patent application No. 20150148623 (Benaron, May 28, 2015, “Hydration Monitoring Sensor and Method for Cell Phones, Smart Watches, Occupancy Sensors, and Wearables”) discloses a sensor for hydration monitoring in and other devices that uses an optional phosphor-coated broadband white LED. U.S. patent application No. 20150148624 (Benaron, May 28, 2015, “Method for Detecting Physiology at Distance or During Movement for Mobile Devices, Illumination, Security, Occupancy Sensors, and Wearables”) discloses a sensor for physiology monitoring in wearables and other devices that uses broadband light transmitted to an ear, face, or wrist.

U.S. patent application No. 20150238083 (Faubert et al., Aug. 27, 2015, “Method and System for Optically Investigating a Tissue of a Subject”) discloses a probe device for optically investigating a person's body tissue comprising: a first probe element, a second probe element, and a third probe element each to be positioned at a respective vertex of a triangle for sensing the tissue. U.S. patent application No. 20150094551 (Frix et al., Apr. 2, 2015, “Continuous Transdermal Monitoring System and Method”) discloses methods and systems for continuous transdermal monitoring via detecting light reflected by body tissue. U.S. patent application No. 20150015888 (Gulati et al., Jan. 15, 2015, “Dynamic Radially Controlled Light Input to a Noninvasive Analyzer Apparatus and Method of Use Thereof”) discloses an analyzer apparatus and method to dynamically irradiate a sample with incident light, wherein the incident light is varied in time in terms of any of: position, radial position relative to a point of the skin of a subject, solid angle, incident angle, depth of focus, energy, and/or intensity.

U.S. Pat. No. 10,893,833 (Haverinen et al., Jan. 19, 2021, “Wearable Electronic Device and Method for Manufacturing Thereof”) discloses a wearable electronic device with a body part made from non-ceramic material, having an inner surface and an outer surface, wherein at least one cavity having a depth is arranged on the inner surface of the body part. U.S. patent application 20240410725 (Huopana, Dec. 12, 2024, “Device for Measurements for a Wearable Device Sensor”) discloses a device comprising: a support configured to receive an appendage of a human body; at least one light source; at least one light detector; and an actuator configured to move the at least one light source, or the at least one light detector, or both to at least two different measurement positions in relation to the support.

U.S. patent application No. 20230190197 (Huttunen, Jun. 22, 2023, “Adjustable Sensor in Wearable Device”) discloses a wearable device with a sensor adjustment mechanism which moves the sensor with respect to a contact surface. U.S. patent application No. 20240293084 (Huttunen et al., Sep. 5, 2024, “Flexible Wearable Ring Device”) discloses a wearable device constructed from elastically deformable flexible materials. U.S. patent application No. 20240122548 (Kangas et al., Apr. 18, 2024, “Techniques for Adaptive Sensors of a Wearable Device”) discloses methods, systems, and devices for adaptive sensors which acquire physiological data from a user via multiple optical channels. U.S. patent application No. 20150216454 (Kasahara et al., Aug. 6, 2015, “Biological Information Measurement Apparatus and Biological Information Measurement Method”) discloses a blood glucose level measurement apparatus which can be mounted on a person's wrist and performs measurement using light.

U.S. patent application No. 20250000218 (Lamsa et al., Jan. 2, 2025, “Wearable Ring Device with Deformable Inner Cover”) discloses a wearable ring device with a housing with one or more sensors which acquire physiological data from a user and a deformable material extending along one or more portions of an inner circumferential surface of the housing. U.S. Pat. No. 8,961,415 (LeBoeuf et al., Feb. 24, 2015, “Methods and Apparatus for Assessing Physiological Conditions”) discloses a monitoring apparatus and methods for assessing a physiological condition of a subject via a portable monitoring device associated with the subject. U.S. patent application No. 20150126824 (LeBoeuf et al., May 7, 2015, “Apparatus for Assessing Physiological Conditions”) discloses a monitoring apparatus and methods for assessing assessment of a physiological condition of a subject using at least two types of physiological information.

U.S. patent applications 20250009085 (Makinen, Jan. 9, 2025, “C-Ring Form Factor for Wearable Ring Device with Adjustable Size”) and 20250009086 (Makinen et al., Jan. 9, 2025, “Adaptive Rigid and Conformable Wearable Ring Device with Adjustable Circumference”) disclose a wearable ring device which transitions between multiple discrete ring sizes. U.S. patent application No. 20240237904 (Makinen et al., Jul. 18, 2024, “Techniques for Measurement Path Multiplexing for a Wearable Device”) discloses methods, systems, and devices for measurement multiplexing for a wearable device. U.S. Pat. No. 9,061,899 (Rowe et al., Jun. 23, 2015, “Apparatus and Method of Biometric Determination Using Specialized Optical Spectroscopy Systems”) discloses methods and apparatuses for performing biometric determinations via optical spectroscopy using light sources such as light emitting diodes, laser diodes, vertical cavity surface emitting lasers, and broadband sources with multiple narrow-band optical filters.

U.S. patent application No. 20150099943 (Russell, Apr. 9, 2015, “Wearable Physiological Sensing Device with Optical Pathways”) discloses a wearable physiological sensing device with at least one light source, a first light pipe coupled with the at least one light source, the first light pipe at least partially circumscribing an extremity of a patient, and including at least one aperture for radiating light from the light source into the extremity. U.S. Pat. No. 9,037,204 (Schlottau, May 19, 2015, “Filtered Detector Array for Optical Patient Sensors”) discloses optical patient monitoring systems which emit two or more wavelengths of light into the tissue of a patient. U.S. patent application 20230113714 (Vallius et al., Apr. 13, 2023, “Configurable Photoplethysmogram System”) discloses wearable devices for optical signal measurement which activate a combination of optical sensors positioned under a protrusion on an inner surface of the device.

U.S. patent application No. 20150220109 (von Badinski et al., Aug. 6, 2015, “Wearable Computing Device”), U.S. patent application No. 20160246326 (von Badinski et al., Aug. 25, 2016, “Wearable Computing Device”), U.S. patent application No. 20170235332 (von Badinski et al., Aug. 17, 2017, “Wearable Computing Device”), U.S. Pat. No. 10,139,859 (von Badinski et al., Nov. 27, 2018, “Wearable Computing Device”), and U.S. Pat. No. 10,156,867 (von Badinski et al., Dec. 18, 2018, “Wearable Computing Device”) disclose a wearable computing device in the form of a ring that can be worn on a person's finger.

U.S. Pat. No. 11,599,147 (von Badinski et al., Mar. 7, 2023, “Wearable Computing Device”), U.S. Pat. No. 11,868,178 (von Badinski et al., Jan. 9, 2024, “Wearable Computing Device”), and U.S. Pat. No. 11,868,179 (von Badinski et al., Jan. 9, 2024, “Wearable Computing Device”) disclose a smart ring with a curved housing having a U-shape interior which stores components including a curved battery, a semi-flexible PCB, and a motion sensor. U.S. patent application No. 20240126329 (von Badinski et al., Apr. 18, 2024, “Wearable Computing Device”), U.S. patent application No. 20240143028 (von Badinski et al., May 2, 2024, “Wearable Computing Device”), and U.S. Pat. No. 12,013,725 (von Badinski et al., Jun. 18, 2024, “Wearable Computing Device”) disclose a finger-worn wearable ring device with a ring-shaped housing, a printed circuit board, and a sensor module with one or more light-emitting components and one or more light-receiving components.

U.S. patent applications 20090018420 (White, Jan. 15, 2009, “Apparatus for Non-Invasive Spectroscopic Measurement of Analytes, and Method of Using the Same”) and 20100249546 (White, Sep. 30, 2010, “Apparatus for Non-Invasive Spectroscopic Measurement of Analytes, and Method of Using the Same”) disclose an apparatus for spectroscopic evaluation of a person's body fluids at the interstitial region, adjacent to or in between a subject's extremities.

### SUMMARY OF THE INVENTION

This invention comprises smart rings with a plurality of light emitters and light receivers which are worn on a person's finger and collect biometric information concerning the person's health. Light from the light emitters is received by the light receivers after the light has been reflected by and/or transmitted through the person's finger. Biometric information is obtained by analyzing changes in the light caused by interaction between the light and the person's finger. In an example, the values of biometric parameters can be obtained by analyzing changes in spectral distribution and/or intensity of the light caused by interaction between the light and the person's finger. These biometric parameters can be selected from the group comprising: oxygenation level, heart rate and heart rate variability, blood pressure level, tissue hydration level, and glucose level.

Although smart rings are useful for obtaining biometric information, there are challenges and limitations with current ring designs. For example, the circumference of a person's finger can change over time due to water retention or loss, weight gain or loss, and other factors. This variation over time can create gaps between optical sensors on a ring and a person's finger which, in turn, cause measurement errors in biometric information. Several of the ring designs disclosed herein address this problem via innovative compressible, expandable, and/or adjustable-size components. As another challenge, unintentional rotation of a finger ring can cause optical sensors to shift away from their preferred measurement locations on the circumference of a finger which, in turn, can also cause measurement errors. Several of the ring designs disclosed herein address this problem via innovative movable components which change the circumferential locations and/or angles at which light beams from light emitters exit the ring toward a person's finger.

### BRIEF INTRODUCTION TO THE FIGURES

FIG. 1 shows a ring with optical sensor sets having two light emitters and one light receiver. FIG. 2 shows a ring with optical sensor sets having a light emitter and two light receivers.

FIG. 3 shows two rings: one with ventral-to-dorsal optical sensor symmetry and one with right-to-left optical sensor symmetry.

FIG. 4 shows a ring with optical sensor sets having light emitters around a light receiver.

FIG. 5 shows a ring with optical sensor sets having light receivers around a light emitter.

FIG. 6 shows a ring with pairs of light emitters and light receivers on lines which are orthogonal to the ring circumference.

FIG. 7 shows a ring with concave indentations on the inner circumference of the ring and light receivers in the indentations.

FIG. 8 shows two rings with circular outer circumferences and oblate circular inner circumferences.

FIG. 9 shows a ring with an arcuate array of compressible components on a portion of the ring's inner circumference.

FIG. 10 shows a ring with light emitters and light receivers being diametrically-opposite compressible components.

FIG. 11 shows a ring with gaps in an inner low-durometer ring and optical sensors in the gaps.

FIG. 12 shows a ring with expandable components on the ring's inner circumference.

FIG. 13 shows a ring with light emitters on expandable components.

FIG. 14 shows a ring with light emitters and light receivers on circumferential undulations.

FIG. 15 shows a ring with light emitters and light receivers being diametrically-opposite expandable components.

FIG. 16 shows a ring with a piezoelectric expandable component.

FIG. 17 shows a ring with an expandable middle layer.

FIG. 18 shows a ring with an expandable bladder on the inner circumference.

FIG. 19 shows a ring with an expandable bladder on the inner circumference which is filled with a flowable substance and a flowable substance reservoir on the outer circumference.

FIG. 20 shows a helical ring with ball-shaped components on its ends.

FIG. 21 shows a helical ring with a flexible ventral segment.

FIG. 22 shows a helical ring with rigid arcuate segments and flexible arcuate segments.

FIG. 23 shows a helical ring with a flexible and/or elastic inner layer or covering.

FIG. 24 shows a ring with two rigid arcuate segments and two flexible arcuate segments.

FIG. 25 shows a ring with a dorsal flexible segment and a ventral hinge or joint.

FIG. 26 shows a ring with telescoping rigid segments and flexible segments.

FIG. 27 shows a telescoping ring with a flexible and/or elastic inner ring or layer.

FIG. 28 shows a ring wherein the ends of a dorsal segment slide into the ends of the rest of the ring.

FIG. 29 shows a ring wherein the ends of a dorsal segment are inserted and connected within the ends of the rest of the ring by springs.

FIG. 30 shows a ring wherein the ends of a dorsal segment are inserted and connected within the ends of the rest of the ring by helical threads.

FIG. 31 shows a ring wherein the ends of a dorsal segment are inserted and connected within the ends of the rest of the ring by actuators.

FIG. 32 shows a ring with an alternating sequence of telescoping segments.

FIG. 33 shows a ring with a convex array of interdigitating and/or interlocking segments connected by a cable, wire, cord, string, chain, band, or filament.

FIG. 34 shows a ring with a convex array of interdigitating and/or interlocking segments connected by a cable, wire, cord, string, chain, band, or filament and covered by flexible outer and inner layers.

FIG. 35 shows a discontinuous ring whose inner circumference is changed by rotating a dorsal bezel or dial.

FIG. 36 shows an adjustable-size ring with rotatable outer and inner undulating rings or layers.

FIGS. 37 and 38 show two sequential views of a ring whose size is adjusted by rotation of an inserted key or other tool.

FIG. 39 shows a ring whose size is adjusted by rotation of an asymmetric segment.

FIG. 40 shows an adjustable-size ring comprising two rotatable non-circular (sub) rings.

FIG. 41 shows a ring with a piezoelectric component between a light emitter and a light receiver.

FIG. 42 shows a ring with one or more actuators between a light emitter and a light receiver.

FIG. 43 shows a ring with a light emitter which is moved along the ring's circumference by a solenoid or piston.

FIG. 44 shows a ring with a light emitter which is moved along the ring's circumference by rotation of a helical thread.

FIG. 45 shows a ring with light emitters which are moved radially (inward or outward) by solenoids or pistons.

FIG. 46 shows a ring with a light emitter moving along a track around a light receiver.

FIG. 47 shows a ring with a light receiver moving along a track around a light emitter.

FIG. 48 shows a ring with rotating optical sensor sets.

FIG. 49 shows a ring comprising two parallel rotating (sub) rings, wherein one (sub) ring has a light emitter and one (sub) ring has a light receiver.

FIGS. 50 and 51 show sequential views of a ring with two circumferential tracks along which a light emitter and a light receiver, respectively, move.

FIGS. 52 through 54 show three sequential views of a ring with a motion sensor and an inner circumferential rotating ring having a light emitter and a light receiver.

FIG. 55 shows a ring comprising two concentric rotating (sub) rings, wherein one (sub) ring has a light emitter and one (sub) ring has a light receiver.

FIG. 56 shows a ring wherein different optical elements in the ring intersect the ring's inner circumference at different angles.

FIG. 57 shows a ring with an undulating inner circumference and optical elements on the undulations.

FIG. 58 shows a ring comprising outer and inner (sub) rings, wherein rotating the outer or inner (sub) ring changes the orientations of light emitters connected to them.

FIG. 59 shows a ring comprising outer and inner (sub) rings, wherein rotating the outer or inner (sub) ring rotates light emitters around central axles of the emitters.

FIG. 60 shows a ring comprising outer and inner (sub) rings, wherein rotating the outer or inner (sub) ring pivots light emitters around their ends.

FIG. 61 shows a ring with a movable reflective component which redirects light from a light emitter, wherein the reflective component is farther from the ring's inner circumference than the light emitter.

FIG. 62 shows a ring with a movable reflective component which redirects light from a light emitter, wherein the reflective component is substantially the same distance from the ring's inner circumference as the light emitter.

FIG. 63 shows a ring with a plurality of movable reflective components which redirect light from a plurality of light emitters.

FIG. 64 shows a ring with a plurality of movable refractive components which redirect light from a plurality of light emitters.

FIG. 65 shows a ring with a plurality of light guides and/or optical fibers which transmit light from a light emitter to different locations on the interior circumference of the ring.

FIG. 66 shows a ring with a plurality of light valves which allow selective transmission of light from a light emitter to different locations on the ring.

FIG. 67 shows a first ring with a light-transmitting pathway which transmits light via total internal reflection and a plurality of light valves.

FIG. 68 shows a second ring with a light-transmitting pathway which transmits light via total internal reflection and a plurality of light valves.

FIG. 69 shows a ring with an inscription which appears when exposed to extreme heat.

FIG. 70 shows a first ring with left-to-right symmetry and dorsal-to-ventral asymmetry in optical sensors.

FIG. 71 shows a second ring with left-to-right symmetry and dorsal-to-ventral asymmetry in optical sensors.

FIG. 72 shows a third ring with left-to-right symmetry and dorsal-to-ventral asymmetry in optical sensors.

FIG. 73 shows a ring with an outer rigid layer, a middle optical-component layer, and an inner compressible layer, wherein the middle optical-component layer includes light emitters, light receivers, movable reflective components, and light barriers.

### DETAILED DESCRIPTION OF THE FIGURES

In an example, a smart ring can comprise: an outer ring which is worn on a person's finger; a plurality of compressible components which collectively span at least one third of the inner circumference of the outer ring; a plurality of light emitters on the outer ring, wherein light emitters are located diametrically-opposite from compressible components; and a plurality of light receivers on the outer ring, wherein light receivers are located diametrically-opposite from compressible components; wherein light from a light emitter is directed toward the person's finger, wherein the light is received by a light receiver after the light has interacted with the person's finger, and wherein biometric information is obtained by analyzing changes in the light caused by interaction between the light and the person's finger.

In an example, light from a light emitter can be received by a light receiver after the light has been reflected by and/or transmitted through the person's finger. In an example, biometric parameter values can be obtained by analyzing changes in spectral distribution and/or intensity in the light caused by interaction between the light and the person's finger. In an example, each light emitter can be directly across the inner diameter of the outer ring from a compressible component. In an example, each light receiver can be directly across the inner diameter of the outer ring from a compressible component. In an example, the outer ring can be made with material having a first Shore or durometer value, wherein the compressible components are made with material having a second Shore or durometer value, and wherein the second value is less than the first value.

In an example, a smart ring can comprise: an outer ring which is worn on a person's finger, wherein the outer ring further comprises one or more first segments with a first average diameter, wherein the outer ring further comprises one or more second segments with a second average diameter which is smaller than the first average diameter, wherein the ends of the one or more second segments are inserted into the ends of the one or more first segments, and wherein the ends of the one or more second segments slide within the ends of the one or more first segments; an inner flexible ring or layer which is closer to the person's finger than the outer ring; one or more light emitters; and one or more light receivers, wherein light from a light emitter is directed toward the person's finger, wherein the light is received by a light receiver after the light has interacted with the person's finger, and wherein biometric information is obtained by analyzing changes in the light caused by interaction between the light and the person's finger.

In an example, light from a light emitter can be received by a light receiver after the light has been reflected by and/or transmitted through the person's finger. In an example, biometric parameter values can be obtained by analyzing changes in spectral distribution and/or intensity in the light caused by interaction between the light and the person's finger. In an example, the ends of a second segment can slide within openings in the ends of a first segment in a telescoping manner. In an example, the ends of a second segment can be tapered. In an example, the ends of a second segment can have smaller diameters than the ends of a first segment, but the middle of the second segment can be the same diameter as that of a first segment.

In an example, a smart ring can comprise: a ring which is worn on a person's finger; wherein the ring further comprises an outer rigid layer which is a first distance from a person's finger, a middle optical-component layer which is a second distance from the person's finger, and an inner compressible layer which is a third distance from the person's finger; wherein the second distance is less than the first distance and the third distance is less than the second distance; wherein the middle optical-component layer further comprises a plurality of light emitters, a plurality of light receivers, a plurality of movable reflective components which reflect light from light emitters toward the person's finger, and a plurality of light barriers between light emitters and light receivers; wherein light from a light emitter is received by a light receiver after the light has interacted with the person's finger, wherein biometric information is obtained by analyzing changes in the light caused by interaction between the light and the person's finger.

In an example, light from a light emitter can be received by a light receiver after the light has been reflected by and/or transmitted through the person's finger. In an example, biometric parameter values can be obtained by analyzing changes in spectral distribution and/or intensity in the light caused by interaction between the light and the person's finger. In an example, a reflective component can be a mirror. In an example, a reflective component can be on a different location on the circumference of the ring than the light emitter whose light the reflective component redirects. In an example, a ring can further comprise a plurality of actuators which move the plurality of reflective components. In an example, a ring can comprise one or more electromagnetic, hydraulic, and/or pneumatic actuators. In an example, a reflective component can be moved by changing an electromagnetic field to which the reflective component is exposed.

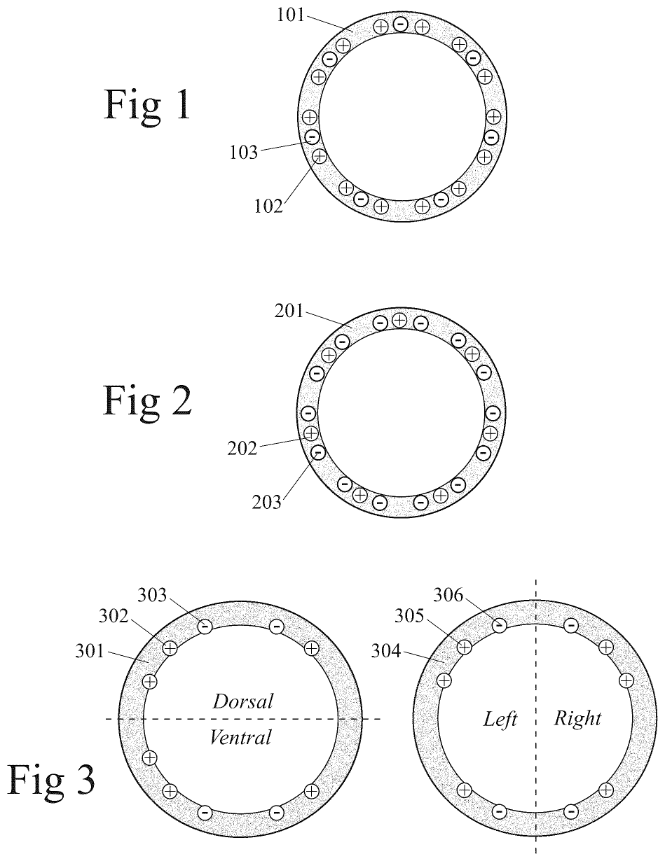

FIG. 1 shows a cross-sectional side view of a smart ring comprising: a finger ring 101 which is worn on a person's finger; a plurality of optical sensor sets on the ring, wherein the optical sensor sets contain light emitters (including light emitter 102) labeled in this figure with plus signs and light receivers (including light receiver 103) labeled in this figure with negative signs, wherein light from a light emitter is directed toward the person's finger, wherein the light is received by a light receiver after the light has interacted with the person's finger (e.g. been reflected by and/or transmitted through the finger), wherein biometric information (e.g. biometric parameter values) is obtained by analyzing changes (e.g. changes in spectral distribution and/or intensity) in the light caused by interaction between the light and the person's finger, wherein there are more light emitters than light receivers in each set, and wherein the average distance between the centers of sets is greater than the average distance between adjacent light emitters and light receivers within a set.

In this example, there are two light emitters and one light receiver in each optical sensor set. In an example, there can be two or more light emitters at different distances from a light receiver in each set. In an example, there can be a plurality of light emitters which emit light of different colors and/or wavelengths, respectively, in each set. In an example, there can be two light emitters which emit light of two different colors and/or wavelengths, respectively, in each set. In an example, there can be three light emitters which emit light of three different colors and/or wavelengths, respectively, in each optical sensor set. In an example, there can be light emitters in each set which emit light in at least three different colors and/or wavelengths selected from the group consisting of: near-infrared, red, green, blue, ultraviolet, broad-range visible. In an example, the same light emitter can emit light of different colors and/or wavelengths at different times.

In an example, light emitters and light receivers in an optical sensor set can all be the same distance from the inner circumferential surface of the ring and/or the surface of the person's finger. In an example, light receivers can be closer than light emitters to the inner circumferential surface of the ring and/or the surface of the person's finger. In an example, the distance between a light emitters and/or light receiver and the inner circumferential surface of the ring and/or the surface of the person's finger can be adjusted by an actuator. In an example, there can be compressible opaque light barriers between light emitters and light receivers. In an example, there can be circular compressible opaque light barriers around light emitters.

In an example, a ring can have at least six optical sensor sets. In an example, a ring can have at least three optical sensor sets. In an example, optical sensor sets can be distributed around the entire circumference of the ring. In an example, sets can collectively span at least half of the circumference of the ring. In an example, there at least one optical sensor set on the dorsal half of the ring. In an example, all sets can have the same number and configuration of light emitters and light receivers. In an example, different sets can have different numbers and/or configurations of light emitters and light receivers. In an example, each set in a subset of optical sensor sets can have more light emitters than those in the rest of the optical sets. In an example, each set on the ventral half of the ring can have more light emitters than those on the dorsal half of the ring. In an example, light emitters and light receivers can be closer together in each set in a subset of optical sensor sets than in the rest of the optical sets. In an example, light emitters and light receivers in each set on the ventral half of the ring can be closer together than those on the dorsal half of the ring.

In an example, the ring can further comprise one or more electromagnetic, hydraulic, and/or pneumatic actuators. In an example, the one or more actuators can move light emitters and/or light receivers. In an example, the actuators can move light emitters and/or light receivers to scan different locations and/or depths of finger tissue at different times. In an example, the actuators can move light emitters and/or light receivers to compensate for unintentional rotation of the ring. In an example, the ring can further comprise mirrors, lenses, and/or prisms which redirect light from the light emitters and/or light receivers. In an example, the mirrors, lenses, and/or prisms can be moved by the one or more actuators. In an example, the ring can comprise a plurality of movable mirrors, lenses, waveguides, and/or prisms which change the vectors of light beams emitted from the light emitters to scan different locations and/or depths of finger tissue at different times. In an example, the ring can comprise a plurality of movable mirrors, lenses, waveguides, and/or prisms which change the vectors of light beams emitted from the light emitters to compensate for unintentional rotation of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 2 shows a cross-sectional side view of a smart ring comprising: a finger ring 201 which is worn on a person's finger; a plurality of optical sensor sets on the ring, wherein the optical sensor sets contain light emitters (including light emitter 202) labeled in this figure with plus signs and light receivers (including light receiver 203) labeled in this figure with negative signs, wherein light from a light emitter is directed toward the person's finger, wherein the light is received by a light receiver after the light has interacted with the person's finger (e.g. been reflected by and/or transmitted through the finger), wherein biometric information (e.g. biometric parameter values) is obtained by analyzing changes (e.g. changes in spectral distribution and/or intensity) in the light caused by interaction between the light and the person's finger, wherein there are more light receivers than light emitters in each set, and wherein the average distance between the centers of sets is greater than the average distance between adjacent light emitters and light receivers within a set.

In this example, there can be two light receivers and one light emitter in each optical sensor set. In an example, there can be two or more light receivers at different distances from a light emitter in each set. In an example, there can be a plurality of light emitters which emit light of different colors and/or wavelengths, respectively, in each set. In an example, there can be two light emitters which emit light of two different colors and/or wavelengths, respectively, in each set. In an example, there can be three light emitters which emit light of three different colors and/or wavelengths, respectively, in each optical sensor set. In an example, there can be light emitters in each set which emit light in at least three different colors and/or wavelengths selected from the group consisting of: near-infrared, red, green, blue, ultraviolet, and broad-range visible. In an example, the same light emitter can emit light of different colors and/or wavelengths at different times.

In an example, light emitters and light receivers in an optical sensor set can all be the same distance from the inner circumferential surface of the ring and/or the surface of the person's finger. In an example, light receivers can be closer than light emitters to the inner circumferential surface of the ring and/or the surface of the person's finger. In an example, the distance between a light emitters and/or light receiver and the inner circumferential surface of the ring and/or the surface of the person's finger can be adjusted by an actuator. In an example, there can be compressible opaque light barriers between light emitters and light receivers. In an example, there can be circular compressible opaque light barriers around light emitters.

In an example, a ring can have at least six optical sensor sets. In an example, a ring can have at least three optical sensor sets. In an example, optical sensor sets can be distributed around the entire circumference of the ring. In an example, sets can collectively span at least half of the circumference of the ring. In an example, there at least one optical sensor set on the dorsal half of the ring. In an example, all sets can have the same number and configuration of light emitters and light receivers. In an example, different sets can have different numbers and/or configurations of light emitters and light receivers. In an example, each set in a subset of optical sensor sets can have more light emitters than those in the rest of the optical sets. In an example, each set on the ventral half of the ring can have more light emitters than those on the dorsal half of the ring. In an example, light emitters and light receivers can be closer together in each set in a subset of optical sensor sets than in the rest of the optical sets. In an example, light emitters and light receivers in each set on the ventral half of the ring can be closer together than those on the dorsal half of the ring.

In an example, the ring can further comprise one or more electromagnetic, hydraulic, and/or pneumatic actuators. In an example, the one or more actuators can move light emitters and/or light receivers. In an example, the actuators can move light emitters and/or light receivers to scan different locations and/or depths of finger tissue at different times. In an example, the actuators can move light emitters and/or light receivers to compensate for unintentional rotation of the ring. In an example, the ring can further comprise mirrors, lenses, and/or prisms which redirect light from the light emitters and/or light receivers. In an example, the mirrors, lenses, and/or prisms can be moved by the one or more actuators. In an example, the ring can comprise a plurality of movable mirrors, lenses, waveguides, and/or prisms which change the vectors of light beams emitted from the light emitters to scan different locations and/or depths of finger tissue at different times. In an example, the ring can comprise a plurality of movable mirrors, lenses, waveguides, and/or prisms which change the vectors of light beams emitted from the light emitters to compensate for unintentional rotation of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 3 shows smart rings with different patterns of optical sensor symmetry. The left portion of FIG. 3 shows an example of smart ring with ventral-to-dorsal symmetry of optical sensors. The right portion of FIG. 3 shows an example of a smart ring with right-to-left symmetry of optical sensors.

The left portion of FIG. 3 shows a cross-sectional side view of a smart ring comprising: a finger ring 301 which is worn on a person's finger; a plurality of light emitters (including light emitter 302) on the ring labeled in this figure with plus signs; a plurality of light receivers (including light receiver 303) on the ring labeled in this figure with negative signs; wherein light from a light emitter is directed toward the person's finger, wherein the light is received by a light receiver after the light has interacted with the person's finger (e.g. been reflected by and/or transmitted through the finger); wherein biometric information (e.g. biometric parameter values) is obtained by analyzing changes (e.g. changes in spectral distribution and/or intensity) in the light caused by interaction between the light and the person's finger; and wherein light emitters and light receivers on the dorsal half of the ring are symmetric to light emitters and light receivers on the ventral half of the ring (e.g. are reflected across the central ventral-to-dorsal axis of the ring).

The right portion of FIG. 3 shows a cross-sectional side view of a smart ring comprising: a finger ring 304 which is worn on a person's finger; a plurality of light emitters (including light emitter 305) on the ring labeled in this figure with plus signs; a plurality of light receivers (including light receiver 306) on the ring labeled in this figure with negative signs; wherein light from a light emitter is directed toward the person's finger, wherein the light is received by a light receiver after the light has interacted with the person's finger (e.g. been reflected by and/or transmitted through the finger); wherein biometric information (e.g. biometric parameter values) is obtained by analyzing changes (e.g. changes in spectral distribution and/or intensity) in the light caused by interaction between the light and the person's finger; and wherein light emitters and light receivers on the left half of the ring are symmetric to light emitters and light receivers on the right half of the ring (e.g. are reflected across the central right-to-left axis of the ring).

In an example, the ring can further comprise one or more electromagnetic, hydraulic, and/or pneumatic actuators. In an example, the one or more actuators can move light emitters and/or light receivers. In an example, the actuators can move light emitters and/or light receivers to scan different locations and/or depths of finger tissue at different times. In an example, the actuators can move light emitters and/or light receivers to compensate for unintentional rotation of the ring. In an example, the ring can further comprise mirrors, lenses, and/or prisms which redirect light from the light emitters and/or light receivers. In an example, the mirrors, lenses, and/or prisms can be moved by the one or more actuators. In an example, the ring can comprise a plurality of movable mirrors, lenses, waveguides, and/or prisms which change the vectors of light beams emitted from the light emitters to scan different locations and/or depths of finger tissue at different times. In an example, the ring can comprise a plurality of movable mirrors, lenses, waveguides, and/or prisms which change the vectors of light beams emitted from the light emitters to compensate for unintentional rotation of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

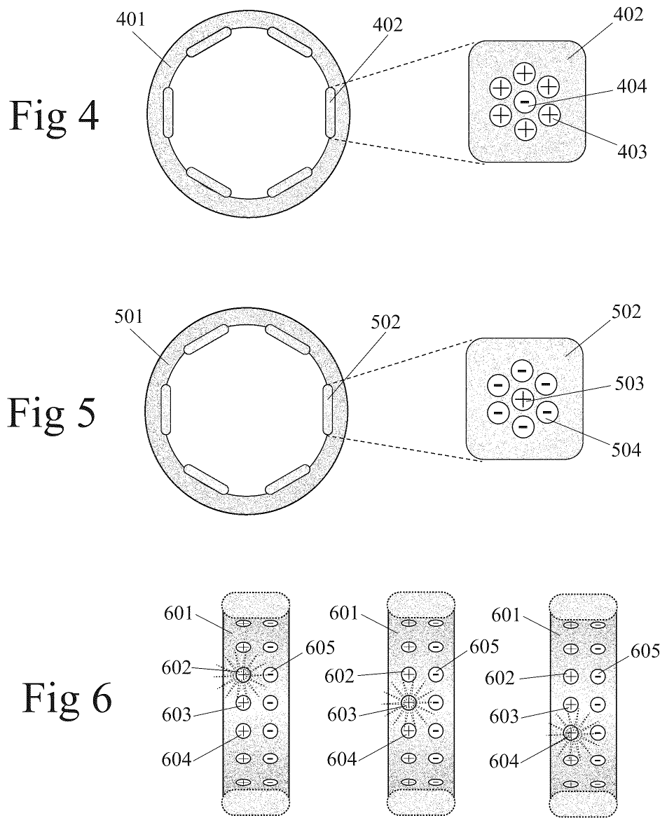

FIG. 4 shows a smart ring comprising: a finger ring 401 which is worn on a person's finger; a plurality of optical sensor sets (including set 402) on the ring, wherein the optical sensor sets contain light emitters (including light emitter 403) labeled in this figure with plus signs and light receivers (including light receiver 404) labeled in this figure with negative signs, wherein light from a light emitter is directed toward the person's finger, wherein the light is received by a light receiver after the light has interacted with the person's finger (e.g. been reflected by and/or transmitted through the finger), wherein biometric information (e.g. biometric parameter values) is obtained by analyzing changes (e.g. changes in spectral distribution and/or intensity) in the light caused by interaction between the light and the person's finger, and wherein there is a plurality of light emitters in an arcuate (e.g. circular) and/or polygonal (e.g. hexagonal) array around a light receiver in each set. The left portion of FIG. 4 shows a cross-sectional side view of this ring. The right portion of FIG. 4 shows an enlarged view of the finger-facing side of one of the optical sensor sets on this ring.

In an example, there can be six light emitters around a central light receiver in each optical sensor set. In an example, a plurality of light emitters can be located at the vertexes of a (virtual) polygon (e.g. a hexagon) around a central light receiver in a set. In an example, there can be four light emitters around a central light receiver in each optical sensor set. In an example, a plurality of light emitters can be located at the vertexes of a (virtual) polygon (e.g. a square) around a central light receiver in a set. In an example, the polygon can be an equilateral polygon (e.g. hexagon or square). In an example, the polygon can have different length sides (e.g. rectangle). In an example, there can be a (circular) compressible opaque light barrier between the light emitters and the light receiver.

In an example, there can be an array of light emitters around a central light receiver in an optical sensor set, wherein the light emitters are all the same distance from the light receiver. In an example, there can be an array of light emitters around a light receiver in an optical sensor set, wherein the light emitters are not all the same distance from the light receiver. In an example, light emitters which emit light of different colors and/or wave lengths can be different distances from the light receiver.

In an example, light emitters in a set can emit light of different colors and/or wavelengths, respectively. In an example, light emitters in each set which emit light in at least three different colors and/or wavelengths selected from the group consisting of: near-infrared, red, green, blue, and ultraviolet. In an example, the same light emitter can emit light of different colors and/or wavelengths at different times. In an example, different light emitters can emit light at different colors and/or wavelengths at different times. In an example, a ring can have at least six optical sensor sets. In an example, a ring can have at least three optical sensor sets. In an example, optical sensor sets can be distributed around the entire circumference of the ring. In an example, sets can collectively span at least half of the circumference of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 5 shows a smart ring comprising: a finger ring 501 which is worn on a person's finger; a plurality of optical sensor sets (including set 502) on the ring, wherein the optical sensor sets contain light emitters (including light emitter 503) labeled in this figure with plus signs and light receivers (including light receiver 504) labeled in this figure with negative signs, wherein light from a light emitter is directed toward the person's finger, wherein the light is received by a light receiver after the light has interacted with the person's finger (e.g. been reflected by and/or transmitted through the finger), wherein biometric information (e.g. biometric parameter values) is obtained by analyzing changes (e.g. changes in spectral distribution and/or intensity) in the light caused by interaction between the light and the person's finger, and wherein there is a plurality of light receivers in an arcuate (e.g. circular) and/or polygonal (e.g. hexagonal) array around a light emitter in each set. The left portion of FIG. 5 shows a cross-sectional side view of this ring. The right portion of FIG. 5 shows an enlarged view of the finger-facing side of one of the optical sensor sets on this ring.

In an example, there can be six light receivers around a central light emitter in each optical sensor set. In an example, a plurality of light receivers can be located at the vertexes of a (virtual) polygon (e.g. a hexagon) around a central light emitter in a set. In an example, there can be four light receivers around a central light emitter in each optical sensor set. In an example, a plurality of light receivers can be located at the vertexes of a (virtual) polygon (e.g. a square) around a central light emitter in a set. In an example, the polygon can be an equilateral polygon (e.g. hexagon or square). In an example, the polygon can have different length sides (e.g. rectangle). In an example, there can be a (circular) compressible opaque light barrier between the light emitters and the light receiver. In an example, the same light emitter can emit light of different colors and/or wavelengths at different times.

In an example, there can be an array of light receivers around a central light emitter in an optical sensor set, wherein the light receivers are all the same distance from the light emitter. In an example, there can be an array of light receivers around a light emitter in an optical sensor set, wherein the light receivers are not all the same distance from the light emitter. In an example, a ring can have at least six optical sensor sets. In an example, a ring can have at least three optical sensor sets. In an example, optical sensor sets can be distributed around the entire circumference of the ring. In an example, sets can collectively span at least half of the circumference of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 6 shows three radial views, at three different times, of an interior portion (e.g. half of the finger-facing inner circumference) a smart ring comprising: a finger ring 601 which is worn on a person's finger; a plurality of light emitters (including light emitters 602, 603, and 604) on the ring labeled in this figure with plus signs; a plurality of light receivers (including light receiver 605) on the ring labeled in this figure with negative signs; wherein light from a light emitter is directed toward the person's finger, wherein the light is received by a light receiver after the light has interacted with the person's finger (e.g. been reflected by and/or transmitted through the finger); wherein biometric information (e.g. biometric parameter values) is obtained by analyzing changes (e.g. changes in spectral distribution and/or intensity) in the light caused by interaction between the light and the person's finger; wherein pairs of light emitters and light receivers are aligned along lines which are orthogonal (or tangential) to the circumference of the ring. The left portion of FIG. 6 shows this view at a first time when only light emitter 602 is activated, the middle portion of FIG. 6 shows this view at a second time when only light emitter 603 is activated, and the right portion of FIG. 6 shows this view at a third time when only light emitter 604 is activated.

In an example, there can be a plurality of pairs of light emitters and light receivers around (a portion of) the circumference of the ring, wherein each pair is aligned along a line which is orthogonal to the circumference of the ring. In an example, there can be sets (with two or more light emitters and one light receiver in each set) around (a portion of) the circumference of a ring, wherein each set is aligned along a line which is orthogonal to the circumference of the ring. In an example, there can be a plurality of pairs of light emitters and light receivers around (a portion of) the circumference of a ring, wherein each pair is aligned along a line which is tangential to the circumference of the ring. In an example, there can be sets (with two or more light emitters and one light receiver in each set) around (a portion of) the circumference of a ring, wherein each set is aligned along a line which is tangential to the circumference of the ring.

In an example, there can be a plurality of triplets of light emitters and light receivers around (a portion of) the circumference of the ring, wherein each triplet is aligned along a line which is orthogonal to the circumference of the ring. In an example, there can be sets (with two or more light emitters and one light receiver in each set) around (a portion of) the circumference of a ring, wherein each set is aligned along a line which is orthogonal to the circumference of the ring. In an example, there can be a plurality of triplets of light emitters and light receivers around (a portion of) the circumference of a ring, wherein each triplet is aligned along a line which is tangential to the circumference of the ring. In an example, there can be sets (with two or more light emitters and one light receiver in each set) around (a portion of) the circumference of a ring, wherein each set is aligned along a line which is tangential to the circumference of the ring.

In an example, the ring can further comprise one or more electromagnetic, hydraulic, and/or pneumatic actuators. In an example, the one or more actuators can move light emitters and/or light receivers. In an example, the actuators can move light emitters and/or light receivers to scan different locations and/or depths of finger tissue at different times. In an example, the actuators can move light emitters and/or light receivers to compensate for unintentional rotation of the ring. In an example, the ring can further comprise mirrors, lenses, and/or prisms which redirect light from the light emitters and/or light receivers. In an example, the mirrors, lenses, and/or prisms can be moved by the one or more actuators. In an example, the ring can comprise a plurality of movable mirrors, lenses, waveguides, and/or prisms which change the vectors of light beams emitted from the light emitters to scan different locations and/or depths of finger tissue at different times. In an example, the ring can comprise a plurality of movable mirrors, lenses, waveguides, and/or prisms which change the vectors of light beams emitted from the light emitters to compensate for unintentional rotation of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

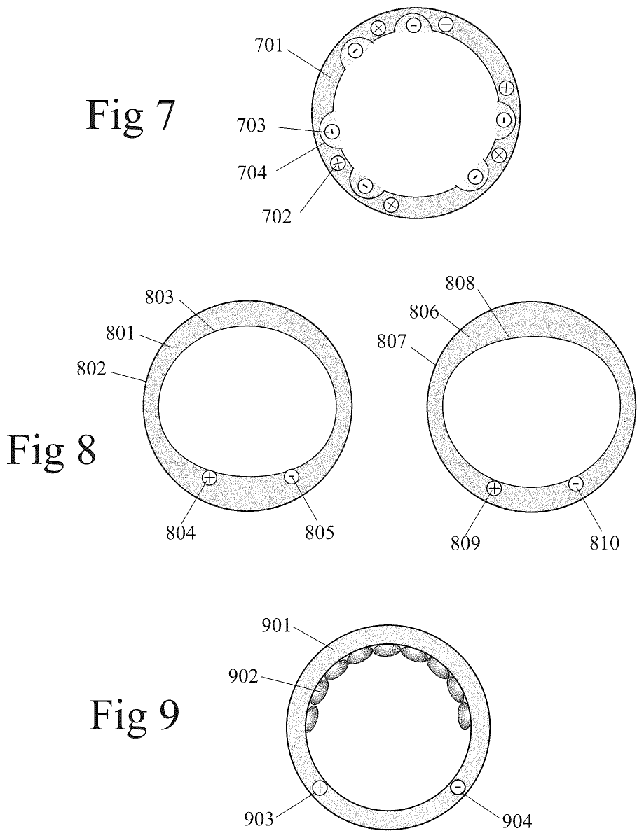

FIG. 7 shows a cross-sectional side view of a smart ring comprising: a finger ring 701 which is worn on a person's finger; a plurality of light emitters (including light emitter 702) on the ring labeled in this figure with plus signs; a plurality of light receivers (including light receiver 703) on the ring labeled in this figure with negative signs; and a plurality of concave indentations or recesses (including indentation or recess 704) on the finger-facing interior circumference of the ring, wherein the light receivers are located in the concave indentations; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, there can be one light receiver in each concave indentation or recess. In an example, the walls of an indentation or recess can be opaque to block light (e.g. serve as a light barrier or shield). In an example, an indentation or recess can have a hemispherical, half-ellipsoidal, or parabolic shape. In an example, the concavity of an indentation or recess can be filled with air. In an example, the concavity of an indentation or recess can be filled with a transparent polymer. In an example, an indentation or recess can comprise a compressible and/or elastomeric polymer. In an example, light emitters can be located in concave indentations or recesses. In an example, light emitters and light receivers can be located in separate concave indentations or recesses.

In an example, a ring can have a compressive opaque inner layer or ring which contacts a person's finger. In an example, there can be concave indentations or recesses in this compressive opaque inner layer or ring. In an example, there can be light emitters and light receivers in the indentations or recesses in the compressive opaque inner layer or ring. In an example, there can be light emitters in a first subset of indentations or recesses in an compressive opaque inner layer or ring and light receivers in a second subset of indentations or recesses in the compressive opaque inner layer or ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 8 shows cross-sectional side views of two smart rings with circular outer circumferences and oblate circular inner circumferences. The left portion of FIG. 8 shows a smart ring whose inner circumference has an oblate circular shape in which a ventral portion of the circle is oblate (e.g. flattened). The right portion of FIG. 8 shows a smart ring whose inner circumference has an oblate circular shape in which a dorsal portion of the circle is oblate (e.g. flattened).

The left portion of FIG. 8 shows a cross-sectional side view of a smart ring comprising: a finger ring 801 which is worn on a person's finger; wherein the finger ring has a circular outer circumference 802 and an oblate circular inner circumference 803, and wherein a ventral portion (e.g. half) of the inner circumference is oblate (e.g. flattened); at least one light emitter 804 on the ring (labeled in this figure with a plus sign); at least one light receiver 805 on the ring (labeled in this figure with a negative sign); wherein light from a light emitter is directed toward the person's finger, wherein the light is received by a light receiver after the light has interacted with the person's finger (e.g. been reflected by and/or transmitted through the finger); wherein biometric information (e.g. biometric parameter values) is obtained by analyzing changes (e.g. changes in spectral distribution and/or intensity) in the light caused by interaction between the light and the person's finger.

The right portion of FIG. 8 shows a cross-sectional side view of a smart ring comprising: a finger ring 806 which is worn on a person's finger; wherein the finger ring has a circular outer circumference 807 and an oblate circular inner circumference 808, and wherein a dorsal portion (e.g. half) of the inner circumference is oblate (e.g. flattened); at least one light emitter 809 on the ring (labeled in this figure with a plus sign); at least one light receiver 810 on the ring (labeled in this figure with a negative sign); wherein light from a light emitter is directed toward the person's finger, wherein the light is received by a light receiver after the light has interacted with the person's finger (e.g. been reflected by and/or transmitted through the finger); wherein biometric information (e.g. biometric parameter values) is obtained by analyzing changes (e.g. changes in spectral distribution and/or intensity) in the light caused by interaction between the light and the person's finger.

In an example, the shape of the inner circumference of a smart ring (which faces toward a person's finger) can be different than the shape of the outer circumference of the smart ring (which faces away from the person's finger). In an example, the inner circumference can be non-circular and the outer circumference can be circular. In an example, the inner circumference can be circular and the outer circumference can be non-circular. In an example, the shape of the inner circumference can be an oblate circle (e.g. flat tire shape) and the outer circumference can be circular. In an example, the shape of the inner circumference can an oblate circle and the outer circumference can be planoconvex, with a flattened portion on the dorsal side of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 9 shows a cross-sectional side view of a smart ring comprising: a finger ring 901 which is worn on a person's finger; an arcuate array (e.g. a sequence, string, and/or chain) of compressible components 902 which span between one-quarter and three-quarters of the inner circumference of the ring; at least one light emitter 903 on the ring (labeled in this figure with a plus sign); at least one light receiver 904 on the ring (labeled in this figure with a negative sign); wherein light from a light emitter is directed toward the person's finger, wherein the light is received by a light receiver after the light has interacted with the person's finger (e.g. been reflected by and/or transmitted through the finger); wherein biometric information (e.g. biometric parameter values) is obtained by analyzing changes (e.g. changes in spectral distribution and/or intensity) in the light caused by interaction between the light and the person's finger.

In an example, the arcuate array of compressible components can be a sequence, string, and/or chain of compressible (e.g. elastomeric) pads, cushions, or beads. In an example, the arcuate array can be a sequence of compressible (e.g. elastomeric) pads, cushions, or beads which are connected together by a filament, cord, string, wire, and/or chain. In an example, compressible components which are closer to the center of the array can be larger than components which are farther from the center of the array. In an example, the arcuate array of compressible components can have a shape which is a section of a circle. In an example, the arcuate array of compressible components can have a shape which is a conic section. In an example, only the ends of the arcuate array of compressible components are connected to the ring.

In an example, the array of compressible components can span between one-quarter and one half of the inner circumference of the ring. In an example, the array of compressible components can span one half of the inner circumference of the ring. In an example, the array of compressible components can span between one half and three-quarters of the inner circumference of the ring. In an example, an arcuate array of compressible components can have a shape which a section of a circle having a different (e.g. larger) diameter than the diameter of the circular shape of (the rest of) the inner circumference of the ring. In an example, the length and/or tension of the array of compressible components can be adjusted.

In an example, an array of compressible components on the inner circumference of a ring can span a location on the inner circumference of a ring which is diametrically-opposite a light emitter and/or light receiver. In an example, all locations on the inner circumference of a ring which are diametrically-opposite light emitters and light receivers on the ring can be spanned by an array of compressible components on the inner circumference of the ring

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

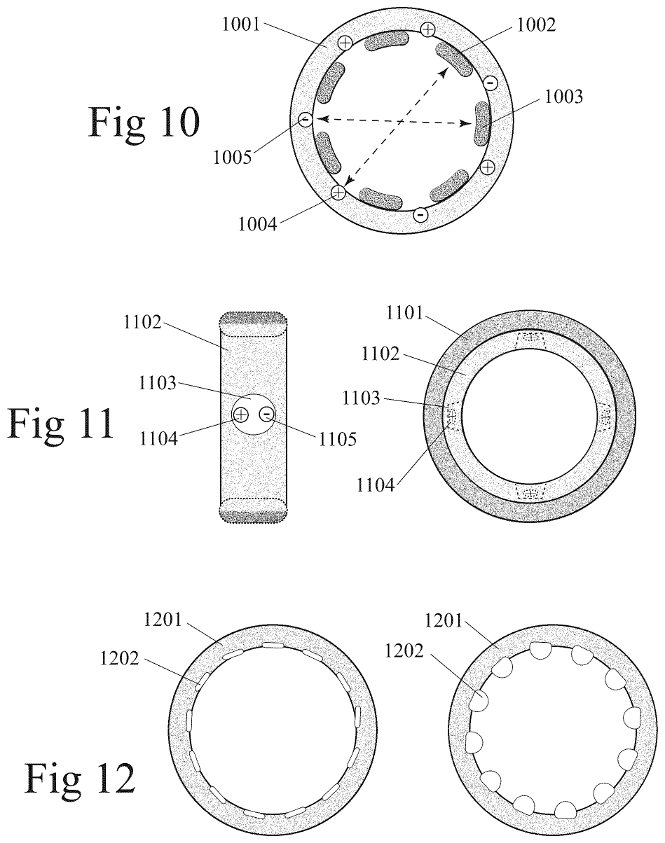

FIG. 10 shows a cross-sectional side view of a smart ring comprising: an outer ring 1001 which is worn on a person's finger; a plurality of compressible components (including compressible components 1002 and 1003) which collectively span at least one third of the inner circumference of the outer ring; a plurality of light emitters (including light emitter 1004) on the outer ring (labeled in this figure with plus signs), wherein the light emitters are located diametrically-opposite (e.g. directly across the inner diameter of the outer ring) from the compressible components; and a plurality of light receivers (including light receiver 1005) on the outer ring (labeled in this figure with negative signs), wherein the light receivers are located diametrically-opposite (e.g. directly across the inner diameter of the outer ring) from the compressible components; wherein light from a light emitter is directed toward the person's finger, wherein the light is received by a light receiver after the light has interacted with the person's finger (e.g. been reflected by and/or transmitted through the finger); wherein biometric information (e.g. biometric parameter values) is obtained by analyzing changes (e.g. changes in spectral distribution and/or intensity) in the light caused by interaction between the light and the person's finger. In this example, light emitter 1004 is diametrically-opposite (e.g. directly across the inner diameter of the outer ring) from compressible component 1002 and light emitter 1005 is diametrically-opposite (e.g. directly across the inner diameter of the outer ring) from compressible component 1003.

In an example, a compressible component can comprise a pad, cushion, bead, or dome made from an elastomeric polymer. In an example, a compressible component can be made from compressible foam. In an example, a compressible component can be made from opaque compressible foam. In an example, a compressible component can comprise a bladder or chamber which is filled with a flowable substance (e.g. gas, liquid, or gel). In an example, the amount of flowable substance in a compressible component can be adjusted to change the size of the component and/or the fit of the ring. In an example, a compressible component can be made with piezoelectric material which expands or contracts when electrical energy is transmitted to it.

In an example the outer ring can be made with material having a first Shore or durometer value and the compressible components can be made with material having a second Shore or durometer value, wherein the second value is less than the first value. In an example, the outer ring can be made with metal and/or a rigid polymer and the compressible components can be made with an elastomeric polymer. In an example, the outer ring can be made with metal and/or a rigid polymer and the compressible components can be made with an elastomeric silicon-base (e.g. PDMS). In an example, the compressible components can have Shore 00 values less than 60.

In an example, each light emitter can be diametrically-opposite a compressible component on the inner circumference of the ring. In an example, each light receivers can be diametrically-opposite a compressible component on the inner circumference of the ring. In an example, a subset of light emitters in a plurality of light emitters can be diametrically-opposite a compressible component on the inner circumference of the ring. In an example, a subset of light receivers in a plurality of light receivers can be diametrically-opposite a compressible component on the inner circumference of the ring. In an example, there can be a portion of compressible component on the inner circumference of the ring diametrically-opposite each light emitter. In an example, there can be a portion of compressible component on the inner circumference of the ring diametrically-opposite each light receiver.

In an example, a ring can comprise at least four compressible components on the inner (e.g. finger-facing) circumference of the ring. In an example, a ring can comprise at least six compressible components on the inner (e.g. finger-facing) circumference of the ring. In an example, there can be at least one compressible component on the dorsal half of the inner circumference of the ring. In an example, there can be at least one compressible component on the dorsal half and at least one compressible component on the ventral half of the inner circumference of the ring. In an example, the pattern of compressible components on the inner circumference of the ring can be symmetric with respect to the left-to-right central axis of the ring. In an example, the pattern of compressible components on the inner circumference of the ring can be symmetric with respect to the dorsal-to-ventral central axis of the ring.

In an example, a compressible component can have a dome shape. In an example, a compressible component can have a hemispherical shape. In an example, a compressible component can have an crescent and/or arc shape. In an example, a compressible component can have an oblate spherical shape. In an example, a compressible component can have an oblong shape. In an example, a compressible component can have a trapezoidal shape. In an example, a compressible component can have an undulating (e.g. sinusoidal) shape. In an example, a compressible component can have a disk shape. In an example, a compressible component can have a planoconvex shape. In an example, a compressible component can have a spherical shape. In an example, a compressible component can have an ellipsoidal shape.

In an example, the center of a compressible component can be thicker (in a radial direction) than the ends of the compressible component. In an example, a compressible component can have a maximum thickness which is between 10% and 40% of the thickness of the outer ring. In an example, a compressible component can have a maximum thickness which is between 25% and 75% of the thickness of the outer ring. In an example, a compressible component can have a maximum thickness which is greater than the thickness of the outer ring. In an example, a compressible component can have a first configuration with a first thickness and a second configuration with a second thickness, wherein the second thickness is between 25% and 75% greater than the second thickness. In an example, a compressible component can be changed from the first configuration to the second configuration by being filled with a flowable substance (e.g. liquid, gas, or gel) or being exposed to electrical current.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

The left portion of FIG. 11 shows a radial view of an interior portion (e.g. half of the finger-facing inner circumference) a smart ring and the right portion of FIG. 11 shows a cross-sectional side view of this smart ring. This smart ring comprises: an outer ring (or circumferential layer) 1101 with a first durometer and/or Shore level; an inner ring (or circumferential layer) 1102 with a second durometer and/or Shore level, wherein the second durometer and/or Shore level is less than the first durometer and/or Shore level, and wherein there are a plurality of gaps, openings, and/or recesses (including 1103) in the inner ring; and a plurality of optical sensor sets in the gaps, openings, and/or recesses, wherein each set further comprises a light emitter (such as 1104), a light receiver (such as 1105, not seen from the perspective shown in the right portion of the figure), or both a light emitter and a light receiver; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 12 shows two views, at two different times, of a smart ring comprising: a finger ring 1201 which is worn on a person's finger; a plurality of expandable components (including expandable component 1202) on the inner (e.g. finger-facing) circumference of the ring, wherein the expandable components are filled with a flowable substance (e.g. a gas, liquid, or gel), wherein the expandable components have an unexpanded (first) configuration in which they are filled with a first amount of the flowable substance, wherein the expandable components have an expanded (second) configuration in which they are filled with a second amount of the flowable substance, and wherein the second amount is greater than the first amount. The left portion of FIG. 12 shows this smart ring at first time, before expandable components have been expanded. The right portion of FIG. 12 shows this smart ring at second time, after expandable components have been expanded.

In an example, this smart ring can further comprise a plurality of light emitters and a plurality of light receivers, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, a ring can comprise at least four expandable components on the inner (e.g. finger-facing) circumference of the ring. In an example, a ring can comprise at least six expandable components on the inner (e.g. finger-facing) circumference of the ring. In an example, a plurality of expandable components can be evenly-distributed around the inner circumference of a ring. In an example, a plurality of expandable components can be evenly-distributed around at least half of the inner circumference of a ring.

In an example, an expandable component can have a dome shape. In an example, an expandable component can have a hemispherical shape. In an example, an expandable component can have an crescent and/or arc shape. In an example, an expandable component can have an oblate spherical shape. In an example, an expandable component can have an oblong shape. In an example, an expandable component can have a trapezoidal shape. In an example, an expandable component can have an undulating (e.g. sinusoidal) shape.

In an example, an expandable component can have a disk shape. In an example, an expandable component can have a planoconvex shape. In an example, an expandable component can have a spherical shape. In an example, an expandable component can have an ellipsoidal shape. In an example, a plurality of expandable components can collectively form an undulating and/or sinusoidal shape.

In an example, an expandable component can comprise a bladder or chamber which is filled with a flowable substance (e.g. gas, liquid, or gel). In an example, the amount of flowable substance in the expandable component can be adjusted to change the size of the component and/or the fit of the ring. In an example, an expandable component can be manually expanded by a person who pumps the flowable substance into it. In an example, the ring can further comprise one or more openings, holes, and/or lumens in the outer circumference of the ring into which the flowable substance can be inserted into (or withdrawn from) one or more expandable components. In an example, an expandable component can be automatically expanded by an pump within the ring which automatically pumps flowable substance into the component.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

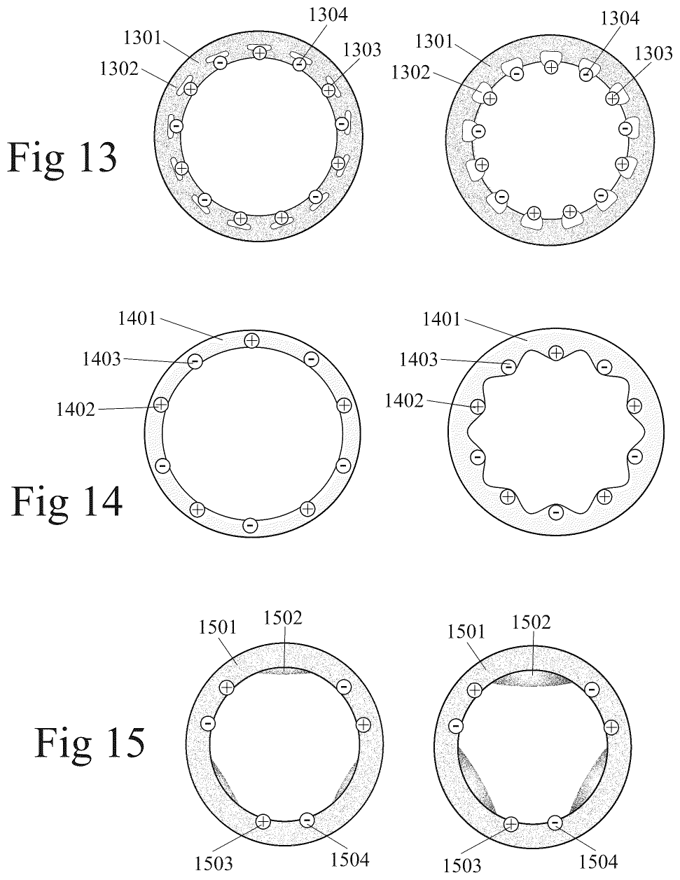

FIG. 13 shows two views, at two different times, of a smart ring comprising: a finger ring 1301 which is worn on a person's finger; a plurality of expandable components (including expandable component 1302), wherein the expandable components are filled with a flowable substance (e.g. a gas, liquid, or gel), wherein the expandable components have an unexpanded (first) configuration in which they are filled with a first amount of the flowable substance, wherein the expandable components have an expanded (second) configuration in which they are filled with a second amount of the flowable substance, and wherein the second amount is greater than the first amount; a plurality of light emitters (including light emitter 1303) on (the finger-facing sides of) the expandable components; and a plurality of light receivers (including light receiver 1304) on (the finger-facing sides of) the expandable components; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

The left portion of FIG. 13 shows this smart ring at first time when the expandable components are in their unexpanded (first) configuration. The right portion of FIG. 13 shows this smart ring at a second time when the expandable components are in their expanded (second) configuration.

In this example, light emitters and/or light receivers are directly on the finger-facing sides of expandable components. In another example, light emitters and/or light receivers are not directly on the finger-facing sides of expandable components, but are nonetheless pushed toward the person's finger by expansion of the expandable components. In an example, a ring can comprise at least four expandable components on the inner (e.g. finger-facing) circumference of the ring. In an example, a ring can comprise at least six expandable components on the inner (e.g. finger-facing) circumference of the ring. In an example, a plurality of expandable components can be evenly-distributed around the inner circumference of a ring. In an example, a plurality of expandable components can be evenly-distributed around at least half of the inner circumference of a ring.

In an example, an expandable component can have a dome shape. In an example, an expandable component can have a hemispherical shape. In an example, an expandable component can have an crescent and/or arc shape. In an example, an expandable component can have an oblate spherical shape. In an example, an expandable component can have an oblong shape. In an example, an expandable component can have a trapezoidal shape. In an example, an expandable component can have an undulating (e.g. sinusoidal) shape.

In an example, an expandable component can have a disk shape. In an example, an expandable component can have a planoconvex shape. In an example, an expandable component can have a spherical shape. In an example, an expandable component can have an ellipsoidal shape. In an example, a plurality of expandable components can collectively form an undulating and/or sinusoidal shape.

In an example, an expandable component can comprise a bladder or chamber which is filled with a flowable substance (e.g. gas, liquid, or gel). In an example, the amount of flowable substance in the expandable component can be adjusted to change the size of the component and/or the fit of the ring. In an example, an expandable component can be manually expanded by a person who pumps the flowable substance into it. In an example, the ring can further comprise one or more openings, holes, and/or lumens in the outer circumference of the ring into which the flowable substance can be inserted into (or withdrawn from) one or more expandable components. In an example, an expandable component can be automatically expanded by an pump within the ring which automatically pumps flowable substance into the component.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 14 shows two views, at two different times, of a smart ring comprising: a finger ring 1401 which is worn on a person's finger, wherein the smart ring has a first configuration with a less-undulating (e.g. substantially-circular) inner (e.g. finger-facing) circumference, wherein the smart ring has a second configuration with a more-undulating (e.g. substantially-sinusoidal) inner (e.g. finger-facing) circumference; a plurality of light emitters (including light emitter 1402) on the inner (e.g. finger-facing) circumference of the ring; and a plurality of light receivers (including light receiver 1403) on the on the inner (e.g. finger-facing) circumference of the ring; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 14 shows this smart ring in its first (less-undulating) configuration. The right portion of FIG. 14 shows this smart ring in its second (more-undulating) configuration.

In an example, light emitters and/or light receivers can be located on inward (finger-facing) undulations or waves of the ring when the ring is in its second configuration. In an example, the ring can be changed from its first configuration to its second configuration, or vice versa, by application of electrical energy to piezoelectric material. In an example, the ring can be changed from its first configuration to its second configuration, or vice versa, by filling bladders and/or chambers with a flowable substance. In an example, the ring can be changed from its first configuration to its second configuration, or vice versa, by activation of a plurality of actuators, solenoids, and/or pistons. In an example, a ring can have four or more undulations or waves in its second configuration. In an example, a ring can have six or more undulations or waves in its second configuration. In an example, a ring can have an inner (finger-facing) circumference with a first degree and/or magnitude of undulation (e.g. waves or protrusions) in a first configuration and a second degree and/or magnitude of undulation (e.g. waves or protrusions) in a second configuration, wherein the second degree and/or magnitude is greater than the first degree and/or magnitude.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 15 shows two views, at two different times, of a smart ring comprising: a finger ring 1501 which is worn on a person's finger; a plurality of expandable components (including 1502) on the inner (finger-facing) circumference of the ring, wherein the expandable components have a first (unexpanded) configuration and a second (expanded) configuration; a plurality of light emitters (including 1503) on the ring; and a plurality of light receivers (including 1504) on the ring; wherein each light emitter and/or light receiver is diametrically-opposite (e.g. across the inner diameter of the ring) from (a portion of) an expandable component; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 15 shows this smart ring in the first configuration. The right portion of FIG. 15 shows this smart ring in the second configuration.

In an example, a ring can comprise at least three expandable components on the inner (e.g. finger-facing) circumference of the ring. In an example, a ring can comprise at least six expandable components on the inner (e.g. finger-facing) circumference of the ring. In an example, a ring can comprise a plurality of expandable components which are evenly-distributed (e.g. equally spaced) around the inner circumference of the ring. In an example, a ring can comprise a plurality of expandable components which are symmetric with respect to the central left-to-right axis of the ring but asymmetric with respect to the central dorsal-to-ventral axis of the ring.

In an example, an expandable component can have a crescent shape, a convex lens shape, a banana shape, an oblate spherical shape, a dome shape, or a disk shape. In an example, a compressible component can have a dome shape. In an example, a compressible component can have a hemispherical shape. In an example, a compressible component can have an crescent and/or arc shape. In an example, a compressible component can have an oblate spherical shape. In an example, a compressible component can have an oblong shape. In an example, a compressible component can have a trapezoidal shape.

In an example, a compressible component can have an undulating (e.g. sinusoidal) shape. In an example, a compressible component can have a disk shape. In an example, a compressible component can have a planoconvex shape. In an example, a compressible component can have a spherical shape. In an example, a compressible component can have an ellipsoidal shape. In an example, the ends of a compressible component can be tapered.

In an example, an expandable component can comprise a bladder or chamber which is filled with a flowable substance (e.g. gas, liquid, or gel). In an example, the amount of flowable substance in the expandable component can be adjusted to change the size of the component and/or the fit of the ring. In an example, an expandable component can be manually expanded by a person who pumps the flowable substance into it. In an example, the ring can further comprise one or more openings, holes, and/or lumens in the outer circumference of the ring into which the flowable substance can be inserted into (or withdrawn from) one or more expandable components. In an example, an expandable component can be automatically expanded by an pump within the ring which automatically pumps flowable substance into the component. In an example, an expandable component can be made with piezoelectric material. In an example, an expandable component can be expanded or contracted by the transmission of electrical energy.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

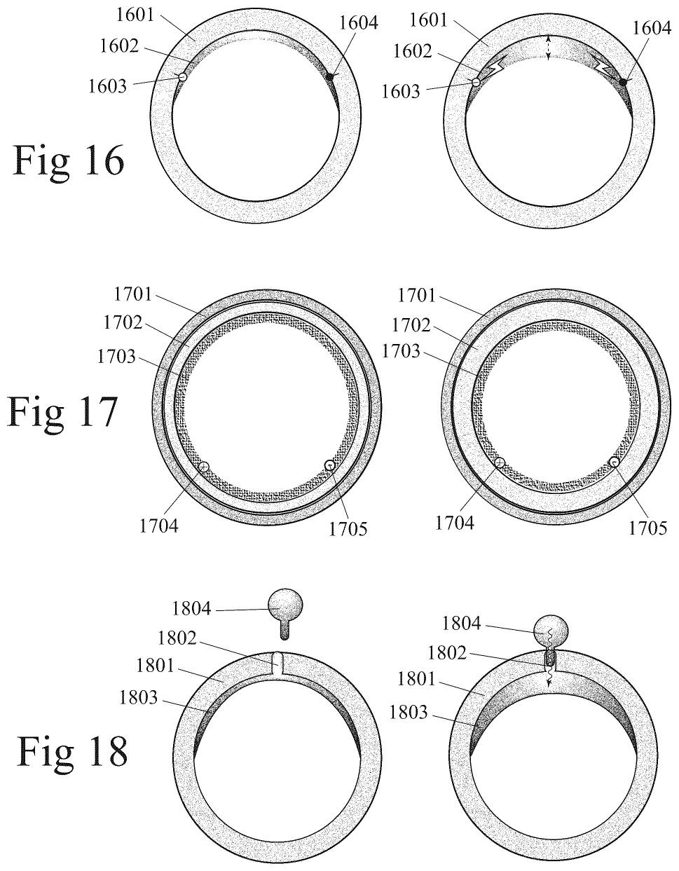

FIG. 16 shows two views, at two different times, of a smart ring comprising: a finger ring 1601 which is worn on a person's finger; a piezoelectric expandable component 1602, wherein the piezoelectric expandable component has an unexpanded (first) configuration and an expanded (second) configuration; and one or more electrodes (1603 and 1604) which transmit electrical energy to the piezoelectric expandable component, wherein transmission of electrical energy from the one or more electrodes to the piezoelectric expandable component changes the component from the unexpanded configuration to expanded configuration, or vice versa. The left portion of FIG. 16 shows this smart ring with the expandable component in the unexpanded configuration. The right portion of FIG. 16 shows this smart ring with the expandable component in the expanded configuration.

In an example, the piezoelectric expandable component can have a crescent shape. In an example, the piezoelectric expandable component can have a shape selected from the group consisting of: crescent shape, arc shape, convex lens shape, banana shape, planoconvex shape, oblate spherical shape, oblong shape, dome shape, and disk shape. In an example, the middle of the piezoelectric component can be thicker than the ends of the piezoelectric component. In an example, the ends of the piezoelectric component can be tapered.

In an example, a piezoelectric expandable component can span between one quarter and one half of the inner (finger-facing) circumference of the ring. In an example, a piezoelectric expandable component can span between 30% and 75% of the inner (finger-facing) circumference of the ring. In an example, a piezoelectric expandable component can span the entire inner circumference of the ring.

In an example, this smart ring can further comprise a plurality of light emitters and a plurality of light receivers, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 17 shows two views, at two different times, of a smart ring comprising: a rigid outer ring (or layer) 1701 which is a first distance from the person's finger; a flexible and/or elastic inner ring 1703 which is a second distance from the person's finger, wherein the second distance is less than the first distance; an expandable middle ring (or layer) 1702 between the rigid outer ring and the flexible and/or elastic inner ring, wherein the expandable middle ring (or layer) has an unexpanded (first) configuration and an expanded (second) configuration; one or more light emitters (including 1704) on the ring; and one or more light receivers (including 1705) on the ring; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 17 shows this smart ring with the middle ring (or layer) in the unexpanded (first) configuration. The right portion of FIG. 17 shows this smart ring with the middle ring (or layer) in the expanded (second) configuration.

In an example, the outer, middle, and inner rings (or layers) can be nested and/or concentric. In an example, the outer ring (or layer) can be metal and the inner ring (or layer) can be a stretchable and/or elastic polymer. In an example, the middle ring (or layer) can comprise piezoelectric material. In an example, application of electrical energy to the middle ring (or layer) changes it from the unexpanded configuration to the second configuration, or vice versa. In an example, the middle ring (or layer) can comprise a bladder or chamber which is filled with a flowable substance (e.g. gas, liquid, or gel). In an example, the amount of flowable substance in the middle ring (or layer) can be adjusted to change the size of the component and/or the fit of the ring. In an example, an middle ring (or layer) can be manually expanded by a person who pumps the flowable substance into it. In an example, the ring can further comprise one or more openings, holes, and/or lumens in the outer circumference of the ring into which the flowable substance can be inserted into (or withdrawn from) the middle ring (or layer). In an example, the middle ring (or layer) can be automatically expanded by an pump within the ring which automatically pumps flowable substance into it.

In an example, light emitters and/or light receivers can be on the middle ring (or layer). In an example, light emitters and/or light receivers can be on the inner ring (or layer). In an example, light emitters and/or light receivers can be pushed toward the surface of the person's finger by expansion of the middle ring (or layer).

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 18 shows two views, at two different times, of a smart ring comprising: a finger ring 1801 which is worn on a person's finger; an expandable bladder (or chamber) 1803 on the inner (e.g. finger-facing) circumference of the ring, wherein the expandable bladder has an unexpanded (first) configuration and an expanded (second) configuration, and wherein the expandable bladder is changed from the unexpanded configuration to the expanded configuration by a flowable substance (e.g. liquid, gas, or gel) being pumped into the expandable bladder; a lumen (e.g. channel) 1802 from the outer circumference of the ring to the expandable bladder, wherein the flowable substance is delivered through the lumen into expandable bladder. In this example, a separate pump (and flowable substance reservoir) 1804 pumps the flowable substance through the lumen to deliver the flowable substance into the expandable bladder. The left portion of FIG. 18 shows this smart ring with the expandable bladder (or chamber) in the unexpanded configuration. The right portion of FIG. 18 shows this smart ring with the expandable bladder (or chamber) in the expanded configuration.

In an example, the expandable bladder (or chamber) can have a crescent shape. In an example, the expandable bladder (or chamber) can have a shape selected from the group consisting of: crescent shape, arc shape, convex lens shape, planoconvex shape, banana shape, oblate spherical shape, oblong shape, dome shape, and disk shape. In an example, an expandable bladder can span between one quarter and one half of the inner (finger-facing) circumference of the ring. In an example, the expandable bladder can span between 30% and 75% of the inner circumference of the ring. In an example, an expandable bladder can span the entire inner circumference of the ring. In an example, a separate pump can be removably inserted into the lumen. In an example, a separate (e.g. detached) pump can be removably attached to the lumen by rotating a helical thread. In an example, a separate (e.g. detached) pump can be removably-connected to the lumen by a snap, clasp, or clip. In an example, the flowable substance can be a gas, liquid, or gel.

In an example, this smart ring can further comprise a plurality of light emitters and a plurality of light receivers, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

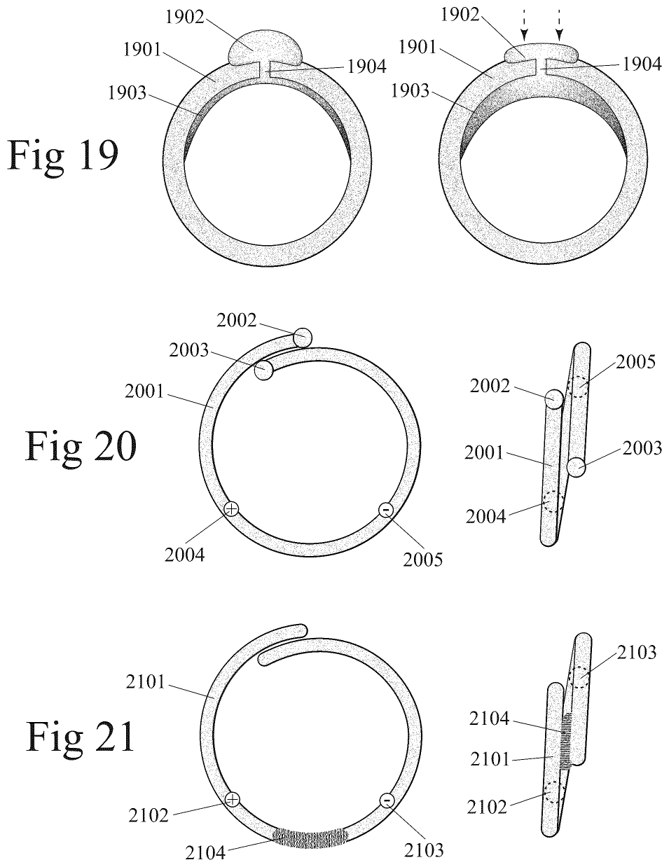

FIG. 19 shows two views, at two different times, of a smart ring comprising: a finger ring 1901 which is worn on a person's finger; an inner bladder (or chamber) 1903 on the inner (e.g. finger-facing) circumference of the ring, wherein the inner bladder has an unexpanded (first) configuration and an expanded (second) configuration, and wherein the inner bladder is changed from the unexpanded configuration to the expanded configuration by pumping a flowable substance into the inner bladder; an outer bladder (or chamber) 1902 on the outer circumference of the ring, wherein the flowable substance can be moved from the outer bladder to the inner bladder through a lumen 1904 to change the inner bladder from the unexpanded configuration to the expanded configuration. The left portion of FIG. 19 shows this smart ring with the inner bladder (or chamber) in the unexpanded configuration. The right portion of FIG. 19 shows this smart ring with the inner bladder (or chamber) in the expanded configuration.

In an example, the flowable substance can be a gas, liquid, or gel. In an example, the flowable substance can be moved (e.g. caused to flow) from the outer bladder to the inner bladder by pressing on the outer bladder. In an example, the flowable substance can be moved (e.g. caused to flow) from the outer bladder to the inner bladder by a pump mechanism. In an example, the ring can further comprise an adjustable valve in the lumen between the outer bladder and the inner bladder which can open or close the lumen. In an example, the outer bladder can be within an ornamental component on the dorsal side of the ring.

In an example, the expandable bladder (or chamber) can have a crescent shape. In an example, the expandable bladder (or chamber) can have a shape selected from the group consisting of: crescent shape, convex lens shape, banana shape, oblate spherical shape, dome shape, and disk shape. In an example, an expandable bladder can span between one quarter and one half of the inner (finger-facing) circumference of the ring. In an example, an expandable bladder can span the entire inner circumference of the ring.

In an example, this smart ring can further comprise a plurality of light emitters and a plurality of light receivers, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 20 shows two views of a smart ring comprising: a helical and/or spiral ring (e.g. coil) 2001 which is worn on a person's finger, wherein the (uncoiled) length of the ring is greater than the circumference of the convex shape (e.g. circle) formed by the helical and/or spiral (coiled) ring; two rounded (e.g. ball-shaped) components 2002 and 2003 which are attached to the ends of the helical and/or spiral ring; one or more light emitters (including 2004) on the ring; and one or more light receivers (including 2005) on the ring; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 20 shows a cross-sectional side view of the ring. The right portion of FIG. 20 shows a dorsal (e.g. top down) view of the ring.

In an example, the (uncoiled) length of the helical and/or spiral ring (e.g. coil) can be 2% to 10% greater than the circumference of the convex shape (e.g. circle) formed by the ring (e.g. coil). In an example, the (uncoiled) length of the helical and/or spiral ring (e.g. coil) can be 2% to 10% greater than the circumference of the person's finger. In an example, the (uncoiled) length of the helical and/or spiral ring (e.g. coil) can be 5% to 20% greater than the circumference of the convex shape (e.g. circle) formed by the ring (e.g. coil). In an example, the (uncoiled) length of the helical and/or spiral ring (e.g. coil) can be 5% to 20% greater than the circumference of the person's finger. In an example, the helical and/or spiral ring (e.g. coil) can span the circumference of the person's finger between 1.02 and 1.20 times.

In an example, the rounded components which are attached to the ends of the helical and/or spiral ring can be convex (e.g. spherical or ellipsoidal). In an example, the rounded components which are attached to the ends of the helical and/or spiral ring can be planoconvex. In an example, the rounded components which are attached to the ends of the helical and/or spiral ring can be bullet-shaped. In an example, the rounded components can be attached to the ends of the ring by rotation of a helical thread. In an example, the ring may not include rounded components if the ends of the helical and/or spiral ring themselves are rounded. In an example, the ring may not include rounded components if the ends of the helical and/or spiral ring themselves are tapered, interdigitating, and/or telescoping.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 21 shows two views of a smart ring comprising: a helical and/or spiral ring (e.g. coil) 2101 which is worn on a person's finger, wherein the (uncoiled) length of the ring (e.g. coil) is greater than the circumference of the convex shape (e.g. circle) formed by the ring, wherein the ring (e.g. coil) includes a flexible and/or bendable segment 2104 on the ventral side of the ring; one or more light emitters (including 2102) on the ring; and one or more light receivers (including 2103) on the ring; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 21 shows a cross-sectional side view of the ring. The right portion of FIG. 21 shows a dorsal (e.g. top down) view of the ring.

In an example, the (uncoiled) length of the helical and/or spiral ring (e.g. coil) can be 2% to 10% greater than the circumference of the convex shape (e.g. circle) formed by the ring (e.g. coil). In an example, the (uncoiled) length of the helical and/or spiral ring (e.g. coil) can be 2% to 10% greater than the circumference of the person's finger. In an example, the (uncoiled) length of the helical and/or spiral ring (e.g. coil) can be 5% to 20% greater than the circumference of the convex shape (e.g. circle) formed by the ring (e.g. coil). In an example, the (uncoiled) length of the helical and/or spiral ring (e.g. coil) can be 5% to 20% greater than the circumference of the person's finger. In an example, the helical and/or spiral ring (e.g. coil) can span the circumference of the person's finger between 1.02 and 1.25 times. In an example, the flexible and/or bendable segment can be made from a flexible polymer and the rest of the helical and/or spiral ring (e.g. coil) can be made from metal. In an example, the flexible and/or bendable segment can span between 5% and 20% of the circumference of the ring (e.g. coil).

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

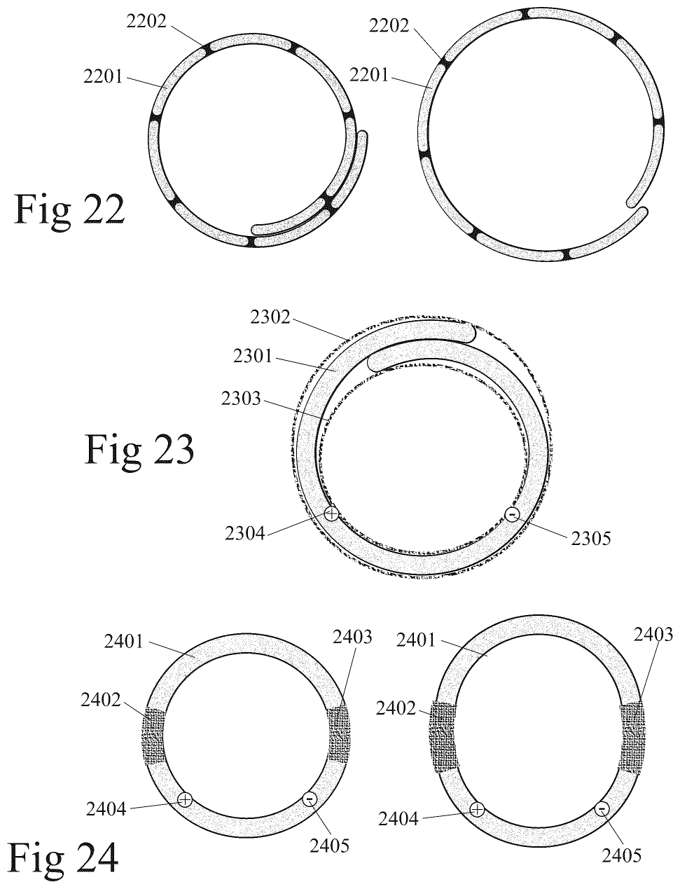

FIG. 22 shows two views, at two different times, of a smart ring comprising: a helical and/or spiral ring (e.g. coil) which is worn on a person's finger, wherein the (uncoiled) length of the ring (e.g. coil) is greater than the circumference of the convex shape (e.g. circle) formed by the ring, and wherein the ring (e.g. coil) further comprises a plurality of rigid arcuate segments (including 2201) and a plurality of flexible arcuate segments (including 2202). In an example, the plurality of flexible arcuate segments enable the ring to expand when a person's finger expands (e.g. due to water retention) or to fit on a larger-diameter finger. The left portion of FIG. 22 shows a cross-sectional side view of the ring with a first diameter. The right portion of FIG. 22 shows a cross-sectional side view of the ring with a larger second diameter.

In an example, the smart ring can further comprise a plurality of light emitters and light receivers, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the smart ring can have at least four rigid arcuate segments and at least four flexible arcuate segments. In an example, the smart ring can have at least six rigid arcuate segments and at least six flexible arcuate segments. In an example, a rigid arcuate segment can be at least twice the length of a flexible arcuate segment. In an example, the rigid arcuate segments can be made from metal and the flexible arcuate segments can be made from a polymer. In an example, the rigid arcuate segments can all be the same length. In an example, rigid arcuate segments on the dorsal half of the ring can be larger than rigid arcuate segments on the ventral half of the ring. In another example, rigid arcuate segments on the ventral half of the ring can be larger than rigid arcuate segments on the dorsal half of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 23 shows a cross-sectional side view of a smart ring comprising: an outer flexible and/or elastic ring, layer, or covering 2302 which is a first distance from the surface of a person's finger; an inner flexible and/or elastic ring, layer, or covering 2303 which is a second distance from the surface of the person's finger, wherein the second distance is less than the first distance; a helical and/or spiral ring (e.g. coil) 2301 which is configured to be worn on the person's finger between the outer flexible and/or elastic ring, layer, or covering and the inner flexible and/or elastic ring, layer, or covering; wherein the (uncoiled) length of the ring (e.g. coil) is greater than the circumference of the convex shape (e.g. circle) formed by the ring; one or more light emitters (including 2304) on the ring; and one or more light receivers (including 2305) on the ring; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the outer flexible and/or elastic ring, layer, or covering and the inner flexible and/or elastic ring, layer, or covering can be different sides of a flexible and/or elastic tubular ring which surrounds the helical and/or spiral ring (e.g. coil). In an example, the outer flexible and/or elastic ring, layer, or covering and the inner flexible and/or elastic ring, layer, or covering can be different sides of a stretchable polymer tube around the helical and/or spiral ring (e.g. coil). In an example, the outer flexible and/or elastic ring, layer, or covering and the inner flexible and/or elastic ring, layer, or covering can be different sides of a stretchable fabric tube around the helical and/or spiral ring (e.g. coil).

In an example, the (uncoiled) length of the helical and/or spiral ring (e.g. coil) can be 2% to 10% greater than the circumference of the convex shape (e.g. circle) formed by the ring (e.g. coil). In an example, the (uncoiled) length of the helical and/or spiral ring (e.g. coil) can be 2% to 10% greater than the circumference of the person's finger. In an example, the (uncoiled) length of the helical and/or spiral ring (e.g. coil) can be 5% to 20% greater than the circumference of the convex shape (e.g. circle) formed by the ring (e.g. coil). In an example, the (uncoiled) length of the helical and/or spiral ring (e.g. coil) can be 5% to 20% greater than the circumference of the person's finger. In an example, the helical and/or spiral ring (e.g. coil) can span the circumference of the person's finger between 1.02 and 1.20 times.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 24 shows two views, at two different times, of a smart ring comprising: a ring which is worn on a person's finger, wherein the ring further comprises a plurality of rigid arcuate segments (including 2401) and a plurality of flexible arcuate segments (including 2402 and 2403); one or more light emitters (2404); and one or more light receivers (2405), wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 24 shows a cross-sectional side view of the ring with a first diameter. The right portion of FIG. 24 shows a cross-sectional side view of the ring with a larger second diameter.

In an example, the ring can have two rigid arcuate segments which are located on the dorsal and ventral sides of the ring and two flexible arcuate segments which are located on the right and left portions of the ring. In an example, the rigid arcuate segments can be larger than the flexible arcuate segments. In an example, the rigid arcuate segments can be at least twice the length of the flexible arcuate segments. In an example, the light emitters and light receivers can be on the rigid arcuate segments. In an example, the rigid arcuate segments can be made from metal and the flexible arcuate segments can be made from a polymer. In an example, the rigid arcuate segments can all be the same length. In an example, rigid arcuate segments on the dorsal half of the ring can be larger than rigid arcuate segments on the ventral half of the ring. In another example, rigid arcuate segments on the ventral half of the ring can be larger than rigid arcuate segments on the dorsal half of the ring. In an example, light emitters and light receivers can be on the rigid arcuate segments.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

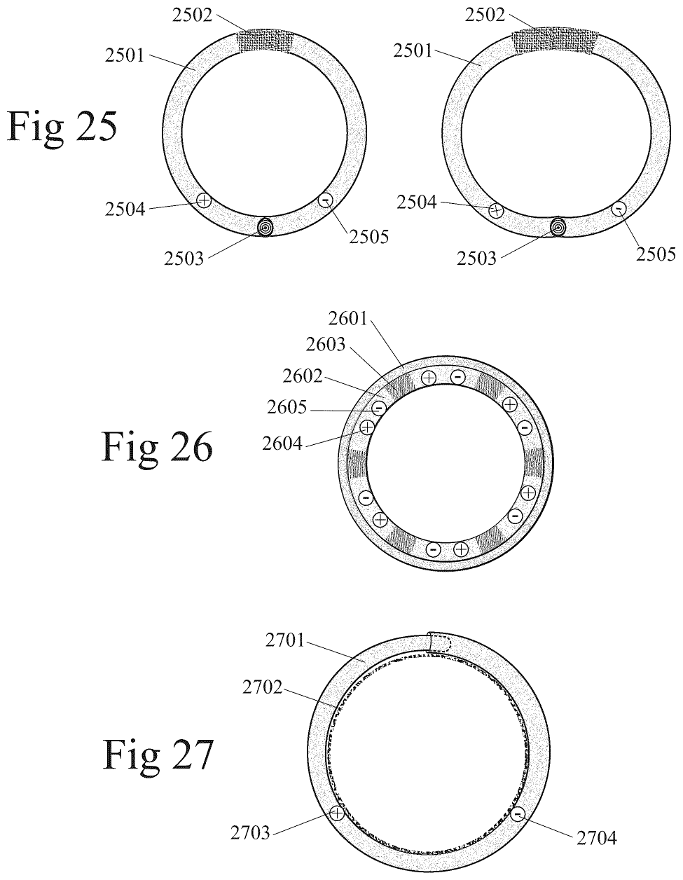

FIG. 25 shows two views, at two different times, of a smart ring comprising: a ring which is worn on a person's finger, wherein the ring further comprises two rigid arcuate segments (including 2501) on the right and left portions of the ring, respectively, wherein the ring further comprises a flexible arcuate segment 2502 on the dorsal side of the ring; wherein the ring further comprises a hinge or joint 2503 on the ventral side of the ring; one or more light emitters 2504; and one or more light receivers 2505, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 25 shows a cross-sectional side view of the ring with a first diameter. The right portion of FIG. 25 shows a cross-sectional side view of the ring with a larger second diameter.

In an example, the rigid arcuate segments can be larger than the flexible arcuate segment. In an example, the rigid arcuate segments can be at least twice the length of the flexible arcuate segment. In an example, the light emitters and light receivers can be on the rigid arcuate segments. In an example, the rigid arcuate segments can be made from metal and the flexible arcuate segment can be made from a polymer. In an example, the rigid arcuate segments can be the same length. In an example, the design of this ring can be reflected across the left-to-right axis, with the flexible arcuate segment being on the ventral side of the ring and the hinge being on the dorsal side of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 26 shows a cross-sectional side view of a smart ring comprising: an outer ring 2601 which is a first distance from a person's finger; an inner ring which is a second distance from the person's finger, wherein the second distance is less than the first distance, wherein the inner ring further comprises a plurality of rigid segments (including 2602) and a plurality of flexible segments (including 2603); a plurality of light emitters (including 2604) on the inner ring; and a plurality of light receivers (including 2605) on the inner ring, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, light emitters can be on the rigid segments of the inner ring. In an example, light receivers can be on the rigid segments of the inner ring. In an example, the outer ring can be a continuous ring and the inner ring can be segmented. In an example, the inner ring can comprise an alternating sequence of rigid segments and flexible segments. In an example, the rigid segments and the flexible segments can interact in a telescoping manner. In an example, the flexible segments can slide into openings in the ends of the rigid segments. In an example, the rigid segments can slide into openings in the ends of the flexible segments.

In an example, the inner ring can comprise at least four rigid segments and four flexible segments. In an example, the inner ring can comprise at least six rigid segments and six flexible segments. In an example, the rigid arcuate segments can be larger (e.g. longer) than the flexible segments. In an example, the rigid arcuate segments can be at least twice the length of the flexible segments. In an example, the rigid arcuate segments can be made from metal and the flexible segments can be made from a polymer.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 27 shows a cross-sectional side view of a smart ring comprising: an outer overlapping and/or telescoping ring 2701 which is worn on a person's finger, wherein a first end of the outer ring has a first diameter, wherein a second end of the outer ring has second diameter which is greater than the first diameter, and wherein the first end is inserted into the second end; an inner flexible and/or elastic ring (or layer) 2702 which is closer to the person's finger than the outer ring; one or more light emitters 2703; and one or more light receivers 2704, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the first end of the outer ring can be inserted into an opening (e.g. central lumen) in the second end of the outer ring. In an example, the first end of the outer ring can slide back and forth within an opening (e.g. central lumen) in the second end of the outer ring, enabling the diameter of the ring to be changed. In an example, the first end of the outer ring can slide within an opening (e.g. central lumen) in the second end of the outer ring in a telescoping manner. In an example, the inner flexible and/or elastic ring (or layer) can be made with an elastomeric polymer. In an example, the inner flexible and/or elastic ring (or layer) can be mode with an elastic and/or stretchable fabric.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

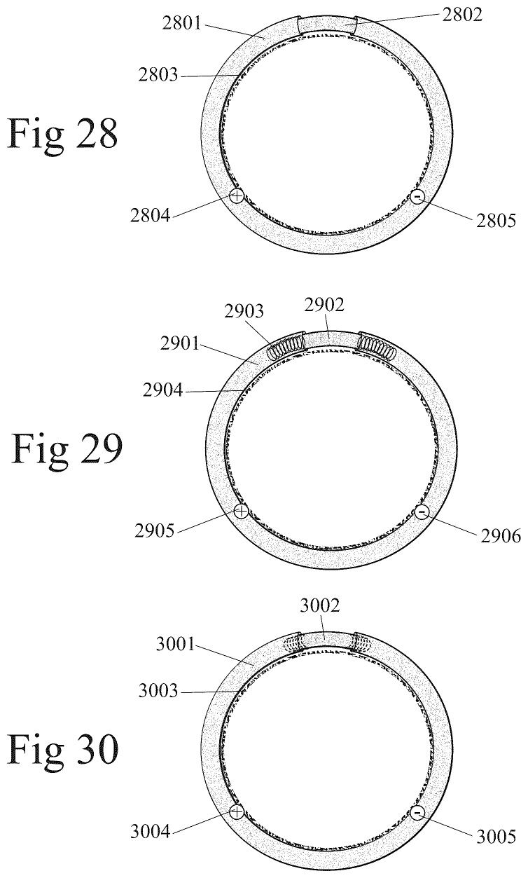

FIG. 28 shows a cross-sectional side view of a smart ring comprising: an outer ring which is worn on a person's finger, wherein the outer ring further comprises one or more first segments 2801 with a first average diameter and one or more second segments 2802 with a second average diameter which is smaller than the first average diameter, wherein the ends of the one or more second segments are inserted into the ends of the one or more first segments, and wherein the ends of the one or more second segments can slide within the ends of the one or more first segments; an inner flexible ring (or layer) 2803 which is closer to the person's finger than the outer ring; one or more light emitters 2804; and one or more light receivers 2805, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ends of a second segment can be inserted into openings (e.g. central lumens) in the ends of a first segment. In an example, the ends of a second segment can slide back and forth within openings (e.g. central lumens) in the end of a first segment, enabling the diameter of the outer ring to be changed. In an example, the ends of a second segment can slide within openings (e.g. central lumens) in the ends of a first in a telescoping manner. In an example, the ends of a second segment can be tapered. In an example, the ends of a second segment can have smaller diameters than the ends of a first segment, but the middle of the second segment can have the same diameter as that of a first segment.

In an example, the outer ring can comprise one first segment and one second segment. In an example, the one or more first segments can be on the dorsal side of the outer ring. In an example, one or more first segments can span between 75% and 95% of the circumference of the outer ring. In an example, one or more second segments can span between 5% and 25% of the circumference of the outer ring. In an example, there can be one second segment on the left side of the outer ring and one second segment on the right side of the outer ring. In an example, there can be one second segment on the dorsal side of the ring and one second segment on the ventral side of the ring.

In an example, the outer ring can further comprise one or more springs which connect the ends of a second segment to a first segment. In an example, the outer ring can further comprise one or more longitudinal elastic members (e.g. elastic bands) which connect the ends of a second segment to a first segment. In an example, the outer ring can further comprise one or more solenoids or pistons which connect the ends of a second segment to a first segment. In an example, the outer ring can further comprise one or more electromagnetic actuators which connect the ends of a second segment to a first segment. In an example, the outer ring can further comprise one or more magnets which connect the ends of a second segment to a first segment. In an example, there can be helical threads on the ends of a second segment which connect them to a first segment.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 29 shows a cross-sectional side view of a smart ring comprising: an outer ring which is worn on a person's finger, wherein the outer ring further comprises a first segment 2901 with a first average diameter and a second segment 2902 with a second average diameter which is smaller than the first average diameter, wherein the ends of the second segment are inserted into the ends of the first segment, wherein the ends of the second segment can slide within the ends of the first segment, and wherein the ends of the second segment are connected to the first segment by springs (including 2903); an inner flexible ring 2904 which is closer to the person's finger than the outer ring; one or more light emitters 2905; and one or more light receivers 2906, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ends of the second segment can be inserted into openings (e.g. central lumens) in the ends of the first segment. In an example, the ends of the second segment can slide back and forth within openings (e.g. central lumens) in the end of the first segment, enabling the diameter of the outer ring to be changed. In an example, the ends of the second segment can slide within openings (e.g. central lumens) in the ends of the second segment in a telescoping manner. In an example, the springs can be completely or partially within (openings in) the second segment. In an example, the first segment can span between 80% and 95% of the circumference of the outer ring. In an example, the second segment can span between 5% and 20% of the circumference of the outer ring. In an example, the first segment can be on the dorsal side of the outer ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 30 shows a cross-sectional side view of a smart ring comprising: an outer ring which is worn on a person's finger, wherein the outer ring further comprises a first segment 3001 with a first average diameter and a second segment 3002 with a second average diameter which is smaller than the first average diameter, wherein the ends of the second segment are helically threaded, and wherein the ends of the second segment are inserted into the ends of the first segment by rotating the helical threads; an inner flexible ring 3003 which is closer to the person's finger than the outer ring; one or more light emitters 3004; and one or more light receivers 3005, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the diameter of the ring can be changed by rotating the helically-threaded second segment. In an example, the diameter of the ring can be adjusted by changing the extent to which the ends of second segment are inserted into the ends of the first segment by rotating the helically-threaded second segment. In an example, the first segment can span between 80% and 95% of the circumference of the outer ring. In an example, the second segment can span between 5% and 20% of the circumference of the outer ring. In an example, the first segment can be on the dorsal side of the outer ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

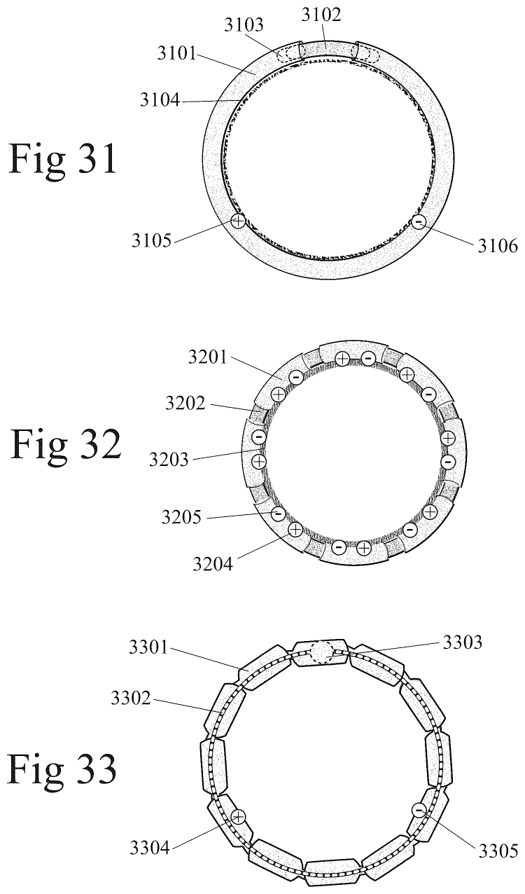

FIG. 31 shows a cross-sectional side view of a smart ring comprising: an outer ring which is worn on a person's finger, wherein the outer ring further comprises a first segment 3101 with a first average diameter and a second segment 3102 with a second average diameter which is smaller than the first average diameter, wherein the ends of the second segment are inserted into the ends of the first segment; one or more actuators (including 3103) which change the amount by which the ends of the second segment are inserted into the first segment; an inner flexible ring 3104 which is closer to the person's finger than the outer ring; one or more light emitters 3105; and one or more light receivers 3106, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, an actuator can be a miniature electromagnetic motor. In an example, an actuator can be a solenoid. In an example, an actuator can be a hydraulic or pneumatic piston. In an example, the diameter of the ring can be increased or decreased by the one or more actuators by changing the degree to which the ends of the second segment are inserted into the ends of the first segment. In an example, there can be two actuators, one connected to each end of the second segment. In an example, there can be just one actuator connected to only one end of the second segment. In an example, the first segment can span between 75% and 95% of the circumference of the outer ring. In an example, the second segment can span between 5% and 25% of the circumference of the outer ring. In an example, a second segment can be on the dorsal side of the outer ring. In an example, the ends of a second segment can be tapered. In an example, the inner flexible ring can be made from elastic and/or stretchable fabric. In an example, the inner flexible ring can be made from an elastomeric polymer.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 32 shows a cross-sectional side view of a smart ring comprising: an outer ring which is worn on a person's finger, wherein the outer ring further comprises an alternating sequence of telescoping segments including a plurality of smaller-diameter segments (including 3202) whose ends slide into openings in a plurality of larger-diameter segments (including 3201); an inner flexible ring 3203 which is closer to the person's finger than the outer ring; a plurality of light emitters (including 3204); and a plurality of light receivers (including 3205); wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, ends of the smaller-diameter segments can telescopically-slide into openings into ends of the larger-diameter segments. In an example, ends of the smaller-diameter segments can telescopically-slide into central lumens of the larger-diameter segments. In an example, the smaller-diameter segments can be tapered. In an example, the ends of the smaller-diameter segments can be smaller than the ends of the larger-diameter segments, but the middle of the smaller-diameter segments can be the same diameter as the larger-diameter segments. In an example, the ends of the larger-diameter segments can be flared. In an example, the ends of the larger-diameter segments can be larger than the smaller-diameter segments, but the middle of the larger-diameter segments can be the same diameter as the smaller-diameter segments.

In an example, the diameter of the ring can be decreased (or increased) when the smaller-diameter segments slide into (or out of), respectively, the openings in the larger-diameter segments. In an example, the ring can further comprise a plurality of tensile components (such as springs or elastic bands) which connect the ends of the smaller-diameter segments with the larger-diameter segments. In an example, the ring can further comprise an arcuate wire, cable, cord, string, chain, or band which connects the smaller-diameter segments and the larger-diameter segments. In an example, this arcuate wire, cable, cord, string, chain, or band can be elastic and/or stretchable. In an example, the length and/or tension of this arcuate wire, cable, cord, chain, or band can be manually or automatically adjusted.

In an example, the outer ring can comprise four smaller-diameter segments and four larger-diameter segments. In an example, the outer ring can comprise at least six smaller-diameter segments and at least six larger-diameter segments. In an example, the smaller-diameter segments can be arcuate. In an example, the larger-diameter segments can be arcuate. In an example, the inner flexible ring can protect the person's finger from being pinched by movement between the segments. In an example, the inner flexible ring can be made from elastic and/or stretchable fabric. In an example, the inner flexible ring can be made from an elastomeric polymer.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 33 shows a cross-sectional side view of a smart ring comprising: a convex (e.g. circular, oval, or planoconvex) array of interdigitating and/or interlocking segments (including 3301) which is worn on a person's finger; a flexible longitudinal member (e.g. a cable, wire, cord, string, chain, band, or filament) 3302 which connects segments in the array to each other; a length and/or tension adjusting component 3303 which adjusts the length and/or tension of the flexible longitudinal member; one or more light emitters 3304; and one or more light receivers 3305; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the flexible longitudinal member can be a cable. In an example, the flexible longitudinal member can be a cord or string. In an example, the flexible longitudinal member can be a wire or chain. In an example, the flexible longitudinal member can be a filament. In an example, the flexible longitudinal member can be elastic and/or stretchable. In an example, the flexible longitudinal member can made from an elastic polymer. In an example, the flexible longitudinal member can be made from metal (e.g. nitinol). In an example, the flexible longitudinal member can be piezoelectric. In an example, the flexible longitudinal member can be helical (e.g. a spring). In an example, the flexible longitudinal member can pass through central openings and/or lumens in the segments. In an example, the flexible longitudinal member can be free to slide back and forth through openings and/or lumens in the segments.

In an example, the length and/or tension adjusting component can automatically change the length and/or tension of the flexible longitudinal member. In an example, the length and/or tension adjusting component can further comprise an actuator which automatically changes the length and/or tension of the flexible longitudinal member. In an example, the length and/or tension adjusting component can change the size (e.g. diameter) of the ring by changing length and/or tension of the flexible longitudinal member. In an example, the size of the ring can be increased by increasing the length and/or decreasing the tension of the flexible longitudinal member. In an example, the size of the ring can be decreased by decreasing the length and/or increasing the tension of the flexible longitudinal member.

In an example, the length and/or tension adjusting component can further comprise a rotating cylinder (e.g. spool) onto which the flexible longitudinal member is wound or from which the flexible longitudinal member is unwound. In an example, the size of the ring can be increased by unwinding the flexible longitudinal member from the cylinder (e.g. spool) or decreased by winding the flexible longitudinal member onto the cylinder. In an example, the length and/or tension adjusting component can further comprise a solenoid or piston which is attached to the flexible longitudinal member, wherein changes in the length of the solenoid or piston change the length and/or tension of the flexible longitudinal member. In an example, the size of the ring can be increased by extending the solenoid or piston or decreased by contracting the solenoid or piston. In another example, the length and/or tension adjusting component can be made from piezoelectric material to which the flexible longitudinal member is attached, wherein the length and/or tension of the flexible longitudinal member is changed by applying electrical current to the piezoelectric material.

In an example, a first end of an interdigitating and/or interlocking segment can fit into a second end of another interdigitating and/or interlocking segment. In an example, a protrusion on a first end of one segment can fit into an opening and/or recess in a second end of another segment. In an example, the degree of interdigitation between segments in the array can be adjusted by changing the length and/or tension of the flexible longitudinal member. In an example, the array can have a first (smaller-diameter) configuration in which segments in the array interdigitate (e.g. overlap) to a first degree and a second (larger-diameter) configuration in which segments in the array interdigitate (e.g. overlap) to a second degree, wherein the second degree is less than the first degree.

In an example, segments in the array can be interdigitated. In an example, a first end of one segment in the array can interdigitate with (e.g. fit into) a second end of another segment in the array. In an example, a first end of one segment can have a protrusion which fits into an opening and/or recess in a second end of another segment in the array. In an example, segments in the array can each have one end which is convex and one end which is concave, so that the first ends of these segments fits into the second ends of these segments. In an example, a second end of a segment can have the same outline as the first end of the segment, only shifted along the longitudinal axis of the segment, so that the first end of one segment fits into the second end of a same-shaped second segment. In an example, the segments can be shaped like bottles. In an example, the segments can have arcuate longitudinal axes which are arcs of a convex shape (e.g. circle, ellipse, or oval). In an example, there can be at least four interdigitating segments in the array. In an example, there can be at least six interdigitating segments in the array.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

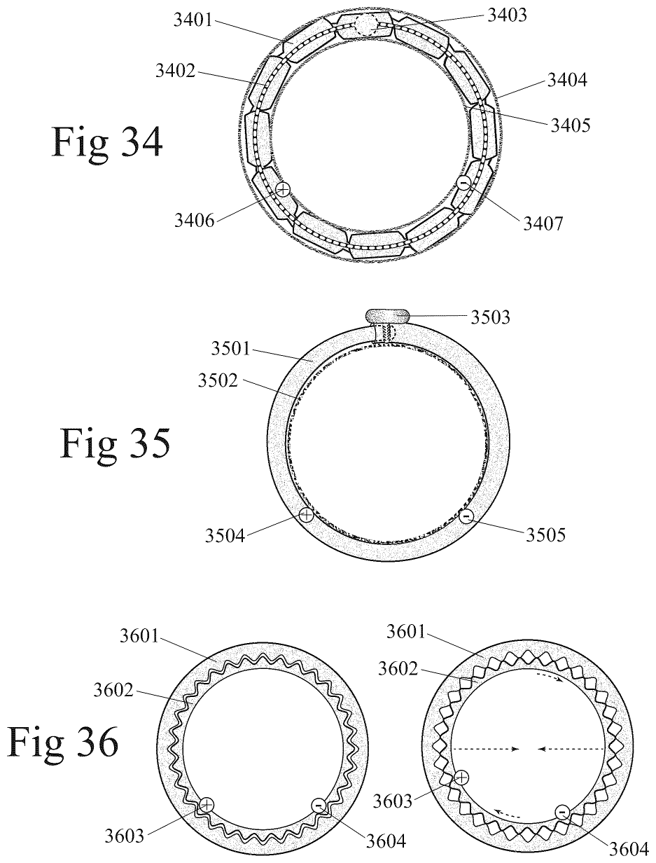

FIG. 34 shows a cross-sectional side view of a smart ring comprising: a convex (e.g. circular, oval, or planoconvex) array of interdigitating and/or interlocking segments (including 3401) which is worn on a person's finger; a flexible longitudinal member (e.g. a cable, cord, string, wire, chain, or filament) 3402 which connects segments in the array to each other; a length and/or tension adjusting component 3403 which adjusts the length and/or tension of the flexible longitudinal member; an outer flexible layer or ring 3404 which is a greater distance from the person's finger than the array; an inner flexible layer or ring 3405 which is a shorter distance from the person's finger than the array; one or more light emitters 3406; and one or more light receivers 3407; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the flexible longitudinal member can be a cable. In an example, the flexible longitudinal member can be a cord or string. In an example, the flexible longitudinal member can be a wire or chain. In an example, the flexible longitudinal member can be a filament. In an example, the flexible longitudinal member can be elastic. In an example, the flexible longitudinal member can made from an elastic polymer. In an example, the flexible longitudinal member can be made from metal (e.g. nitinol). In an example, the flexible longitudinal member can be made from piezoelectric material. In an example, the flexible longitudinal member can be helical (e.g. a spring). In an example, the flexible longitudinal member can pass through central openings and/or lumens in the segments. In an example, the flexible longitudinal member can be free to slide back and forth through openings and/or lumens in the segments.

In an example, the length and/or tension adjusting component can automatically change the length and/or tension of the flexible longitudinal member. In an example, the length and/or tension adjusting component can further comprise an actuator which automatically changes the length and/or tension of the flexible longitudinal member. In an example, the length and/or tension adjusting component can change the size (e.g. diameter) of the ring by changing length and/or tension of the flexible longitudinal member. In an example, the size of the ring can be increased by increasing the length and/or decreasing the tension of the flexible longitudinal member. In an example, the size of the ring can be decreased by decreasing the length and/or increasing the tension of the flexible longitudinal member.

In an example, the length and/or tension adjusting component can further comprise a rotating cylinder (e.g. spool) onto which the flexible longitudinal member is wound or from which the flexible longitudinal member is unwound. In an example, the size of the ring can be increased by unwinding the flexible longitudinal member from the cylinder (e.g. spool) or decreased by winding the flexible longitudinal member onto the cylinder. In an example, the length and/or tension adjusting component can further comprise a solenoid or piston which is attached to the flexible longitudinal member, wherein changes in the length of the solenoid or piston change the length and/or tension of the flexible longitudinal member. In an example, the size of the ring can be increased by extending the solenoid or piston or decreased by contracting the solenoid or piston. In another example, the length and/or tension adjusting component can be made from piezoelectric material to which the flexible longitudinal member is attached, wherein the length and/or tension of the flexible longitudinal member is changed by applying electrical current to the piezoelectric material.

In an example, a first end of an interdigitating and/or interlocking segment can fit into a second end of another interdigitating and/or interlocking segment. In an example, a protrusion on a first end of one segment can fit into an opening and/or recess in a second end of another segment. In an example, the degree of interdigitation between segments in the array can be adjusted by changing the length and/or tension of the flexible longitudinal member. In an example, the array can have a first (smaller-diameter) configuration in which segments in the array interdigitate (e.g. overlap) to a first degree and a second (larger-diameter) configuration in which segments in the array interdigitate (e.g. overlap) to a second degree, wherein the second degree is less than the first degree.

In an example, segments in the array can be interdigitated. In an example, a first end of one segment in the array can interdigitate with (e.g. fit into) a second end of another segment in the array. In an example, a first end of one segment can have a protrusion which fits into an opening and/or recess in a second end of another segment in the array. In an example, segments in the array can each have one end which is convex and one end which is concave, so that the first ends of these segments fits into the second ends of these segments. In an example, a second end of a segment can have the same outline as the first end of the segment, only shifted along the longitudinal axis of the segment, so that the first end of one segment fits into the second end of a same-shaped second segment. In an example, the segments can be shaped like bottles. In an example, the segments can have arcuate longitudinal axes which are arcs of a convex shape (e.g. circle, ellipse, or oval). In an example, there can be at least four interdigitating segments in the array. In an example, there can be at least six interdigitating segments in the array.

In an example, the inner flexible layer or ring can prevent the person's finger from being pinched by movement between the segments. In an example, the outer flexible layer or ring can prevent the edges of the segments from snagging on things in the environment. In an example, the inner and outer flexible layers or rings can be made from an elastic polymer. In an example, the inner and outer flexible layers or rings can be made from a stretchable fabric. In an example, the inner and outer flexible layers or rings can be different sides of the same flexible tubular structure, wherein this flexible tubular structure surrounds the array. In an example, the inner and outer flexible layers or rings can be different sides of the same flexible tubular structure, wherein the array is inside this flexible tubular structure.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 35 shows a cross-sectional side view of a smart ring comprising: an outer overlapping and/or telescoping ring 3501 which is worn on a person's finger, wherein a first end of the ring has a first diameter, wherein a second end of the ring has a second diameter which is greater than the first diameter, and wherein the first end is inserted into the second end; a rotatable dial or bezel 3503 on the dorsal side of the ring, wherein rotating the dial or bezel changes the amount by which the first end of the ring is inserted into the second end of the ring; an inner flexible ring 3502 which is closer to the person's finger than the outer ring; one or more light emitters 3504; and one or more light receivers 3505, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the first end of the ring can be inserted into an opening (e.g. central lumen) in the second end of the ring. In an example, the first end of the ring can slide into a central opening and/or lumen in the second end of the ring, enabling the diameter of the ring to be changed. In an example, the first end of the ring can slide within an opening (e.g. central lumen) in the second end of the ring in a telescoping manner. In an example, the first end of the ring can be tapered. In an example, the second end of the ring can be flared. In an example, the inner flexible ring can be made with an elastomeric polymer. In an example, the inner flexible ring can be mode with an elastic and/or stretchable fabric.

In an example, the first end of the ring can have threads, notches, cogs, gears, or indentations which are engaged when the dial or bezel is rotated, thereby pulling the first end of the ring further into an opening in the second end of the ring, or pushing the first end further out from an opening in the second end of the ring. In an example, the first end of the ring can have threads, notches, cogs, gears, or indentations which engage with threads, notches, cogs, gears, or indentations on the dial or bezel. In an example, rotation of the dial or bezel can rotate a central axle with threads, notches, cogs, gears, or indentation which engage the first end of the ring, thereby changing the amount by which the first end of the ring is inserted into the second end of the ring.

In an example, rotating the dial or bezel changes the amount by which the first end of the ring is inserted into the second end of the ring, thereby changing the size (e.g. diameter) of the ring. In an example, rotating the dial or bezel in a first direction (e.g. clockwise) decreases the amount by which the first end of the ring is inserted into the second end of the ring, thereby increasing the size (e.g. diameter) of the ring. In an example, rotating the dial or bezel in a second direction (e.g. counter-clockwise) increases the amount by which the first end of the ring is inserted into the second end of the ring, thereby decreasing the size (e.g. diameter) of the ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 36 shows two views, at two different times, of a smart ring comprising: an outer ring (or layer) 3601 which is worn on a person's finger at a first distance from the surface of the finger, wherein the side of the outer ring (or layer) which faces toward the person's finger has a first set of portions (e.g. undulations) which protrude out from the ring (or layer) toward the person's finger; an inner ring (or layer) 3602 which is worn on a person's finger at a second distance from the surface of the finger, wherein the second distance is less than the first distance, wherein the side of the inner ring (or layer) which faces away from the person's finger has a second set of portions (e.g. undulations) which protrude out from the ring (or layer) away from the person's finger; wherein the outer ring (or layer) and the inner ring (or layer) have a first configuration in which the first set of portions which protrude toward the person's finger are not aligned with the second set of portions which protrude away from the person's finger and the inner ring (or layer) has a first inner diameter; wherein the outer ring (or layer) and the inner ring (or layer) have a second configuration in which the first set of portions which protrude toward the person's finger are aligned with the second set of portions which protrude away from the person's finger, causing gaps between the outer ring (or layer) and the inner ring (or layer), and causing the inner ring (or layer) to have a second inner diameter, wherein the second inner diameter is less than the first inner diameter; wherein the outer ring (or layer) and the inner ring (or layer) are changed between the first configuration and the second configuration by rotating the outer ring (or layer) relative to the inner ring (or layer), or vice versa; one or more light emitters (3603); and one or more light receivers (3604), wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 36 shows a cross-sectional side view of the ring with a first (smaller) inner diameter. The right portion of FIG. 36 shows a cross-sectional side view of the ring with a second (larger) inner diameter.

In an example, the protruding portions of the outer ring and/or the inner ring can be waves of an undulating surface. In an example, the protruding portions of the outer ring and/or the inner ring can be waves of a sinusoidal surface. In an example, the protruding portions of the outer ring and/or the inner ring can be peaks of a sawtooth and/or zigzag surface. In an example, aligning protrusions on the outer and inner rings can cause them to mesh snugly without gaps, thereby increasing the inner diameter of the ring. In an example, aligning protrusions on the outer and inner rings can create gaps between them, thereby decreasing the inner diameter of the ring. In an example, the inner ring can be flexible and/or compressible.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

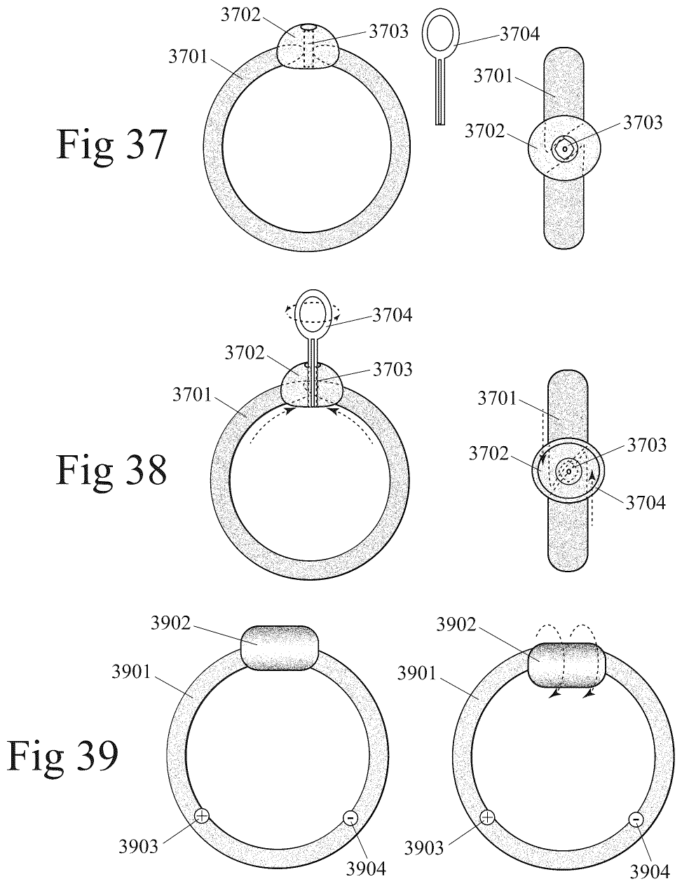

FIGS. 37 and 38 show cross-sectional side views and dorsal (e.g. top-down) views, at two different times, of a smart ring system comprising: a ring 3701 which is worn on a person's finger; a housing 3702 on the dorsal side of the ring; an opening 3703 in the housing; and a rotatable tool (e.g. a key, screwdriver, or Allen wrench) 3704 which can be inserted into the opening and then rotated, wherein rotation of the tool changes the distance between the ends of the ring, thereby changing the diameter of the ring. FIG. 37 shows this system before the tool has been inserted into the ring. FIG. 38 shows this system after the tool has been inserted into the ring and rotated, thereby changing the diameter of the ring. The left portions of FIGS. 37 and 38 show cross-sectional side views of the ring at two different times. The right portions of FIGS. 37 and 38 show dorsal (e.g. top-down) views of the ring at two different times.

In an example, the rotatable tool can be a key. In an example, the rotatable tool can be a screwdriver. In an example, the rotatable tool can be an Allen wrench. In an example, the rotatable tool can directly engage the ends of the ring, thereby pulling them closer together or pushing them farther apart. In an example, the ends of the ring can have threads, notches, cogs, or indentations which are engaged when the rotatable tool is rotated, thereby pulling them closer together or pushing them farther apart. In an example, the ring can further comprise an additional component (such as a cog wheel) in the housing which is between the rotatable tool and the ends of the ring, wherein rotation of the rotatable tool moves (e.g. rotates) the additional component which, in turn, moves the ends of the ring. In an example, rotation of the tool in a first direction (e.g. clockwise) can decrease the diameter of the ring and rotation of the tool in a second direction (e.g. counter-clockwise) can increase the diameter of the ring.

In an example, the ring can further comprise a plurality of light emitters and a plurality of light receivers, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera.

Relevant variations discussed elsewhere in this disclosure or in priority-linked disclosures can also be applied to this example.

FIG. 39 shows two views, at two different times, of a smart ring comprising: a ring 3901 which is worn on a person's finger; a rotatable segment 3902 which rotates asymmetrically around a portion of the ring, wherein the axis around which the rotatable segment rotates is not through the cross-sectional center of the rotatable segment, wherein the rotatable segment has a first configuration which protrudes a first distance from the ring toward the finger, wherein the rotatable segment has a second configuration which protrudes a second distance from the ring toward the ringer, wherein the second distance is greater than the first distance, and wherein the rotatable segment is changed between the first configuration and the second configuration by being rotated; one or more light emitters 3903; and one or more light receivers 3904, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 39 shows a side view of the ring when the rotatable segment is in the first configuration and the ring has a larger inner circumference. The right portion of FIG. 39 shows a side view of the ring when the rotatable segment is in the second configuration and the ring has a smaller inner circumference.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

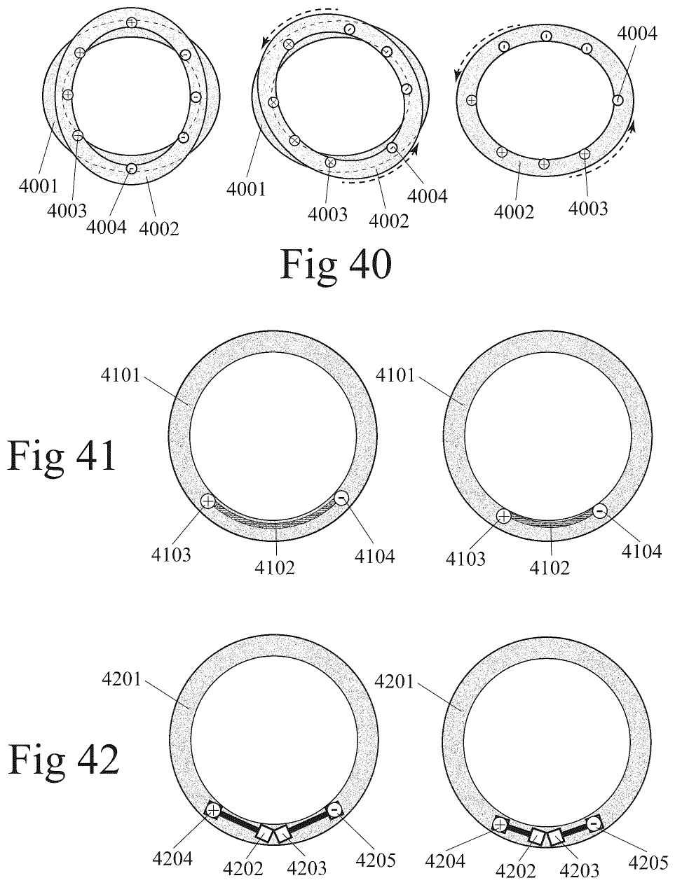

FIG. 40 shows two views, at two different times, of a smart ring comprising: a first non-circular ring 4001 which is worn on a person's finger; a second non-circular ring 4002 which is worn on the person's finger; wherein the first non-circular ring and the second non-circular ring are parallel to each other, wherein the first non-circular ring and the second non-circular ring are rotatably-connected to each other, wherein the first non-circular ring can be rotated relative to the second non-circular ring, or vice versa, wherein the first non-circular ring and the second non-circular ring have a first configuration in which they are not aligned and the intersection of their inner circumferences has a first size, wherein the first non-circular ring and the second non-circular ring have a second configuration in which they are aligned and the intersection of their inner circumferences has a second size which is greater than the first size; a plurality of light emitters (including 4003); and a plurality of light receivers (including 4004), wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 40 shows a side view of the ring when the two non-circular rings are in the first (non-aligned) configuration and the combined ring has a smaller inner circumference. The right portion of FIG. 40 shows a side view of the ring (in which ring 4001 is not seen) when the two non-circular rings are in the second (aligned) configuration and the combined ring has a larger inner circumference.

In an example, a non-circular ring can have an oval or elliptical shape. In an example, a non-circular ring can have a (non-equilateral) polygonal shape. In an example, the first non-circular ring and the second non-circular ring can both have the same non-circular shape. In an example, the first non-circular ring and the second non-circular ring can have different non-circular shapes.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 41 shows two views, at two different times, of a smart ring comprising: a ring 4101 which is worn on a person's finger; a light emitter 4103; a light receiver 4104, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger; and a piezoelectric component (e.g. piezoelectric strip or band) 4102 connected to the light emitter and the light receiver, wherein application of electric current to the piezoelectric member changes the distance between the light emitter and the light receiver. The left portion of FIG. 41 shows a side view of the ring when there is a larger distance between the light emitter and the light receiver. The right portion of FIG. 41 shows a side view of the ring when there is a smaller distance between the light emitter and the light receiver.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 42 shows two views, at two different times, of a smart ring comprising: a ring 4201 which is worn on a person's finger; a light emitter 4204; a light receiver 4205, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger; and one or more actuators (4202 and 4203) connected to the light emitter and the light receiver, wherein the actuators change the distance between the light emitter and the light receiver. The left portion of FIG. 42 shows a side view of the ring when there is a larger distance between the light emitter and the light receiver. The right portion of FIG. 42 shows a side view of the ring when there is a smaller distance between the light emitter and the light receiver.

In an example, an actuator can be an electric motor. In an example, an actuator can be a solenoid. In an example, an actuator can be a hydraulic or pneumatic piston. In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

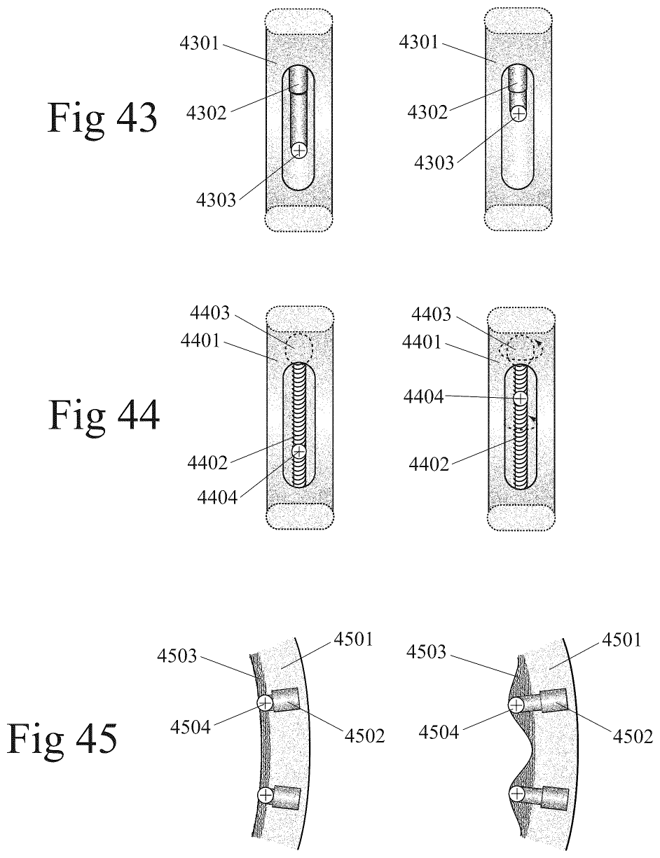

FIG. 43 shows two radial views, at two different times, of a section of the inner circumference of a smart ring comprising: a ring 4301 which is worn on a person's finger; a solenoid or piston 4302; and a light emitter 4303 attached to the solenoid or piston. The left portion of FIG. 43 shows this view at a first time when the solenoid or piston is extended. The right portion of FIG. 43 shows this view at a second time when the solenoid or piston is contracted. Extension or contraction of the solenoid or piston changes the location of the light emitter on the circumference of the ring.

In an example, the ring can further comprise a light receiver, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 44 shows two radial views, at two different times, of a section of the inner circumference of a smart ring comprising: a ring 4401 which is worn on a person's finger; an actuator 4403; a helically-threaded cylinder 4402, wherein the actuator rotates the helically-threaded cylinder; and a light emitter 4404 attached to the helically-thread cylinder, wherein rotation of the cylinder changes the location of the light emitter. The left portion of FIG. 44 shows this view at a first time when the light emitter is in a first location. The right portion of FIG. 44 shows this view at a second time when the light emitter is in a second location.

In an example, the ring can further comprise a light receiver, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 45 shows two cross-sectional side views, at two different times, of a section of the circumference of a smart ring comprising: a ring 4501 which is worn on a person's finger; a plurality of solenoids or pistons (including 4502); a plurality of light emitters (including 4504) attached to the solenoids or pistons, wherein extension or contraction of the solenoids or pistons changes the location (e.g. protrusion from the ring) of the light emitters; and a flexible and/or compressible inner ring layer 4503, wherein extension or contraction of the solenoids or pistons changes the configuration (e.g. thickness and/or undulation) of the inner ring layer. The left portion of FIG. 44 shows this view at a first time when the solenoids or pistons are contracted. The right portion of FIG. 44 shows this view at a second time when the solenoids or pistons are extended.

In an example, the ring can further comprise a light receiver, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, extension of the solenoids or pistons moves the light emitters closer toward the person's finger. In an example, contraction of the solenoids or pistons moves the light emitters farther away from the person's finger. In an example, extension of the solenoids or pistons expands the thickness of the inner ring layer in proximity to the light emitters. In an example, extension of the solenoids or pistons expands the thickness of the inner ring layer in proximity to the light emitters so that the light emitters are less likely to poke uncomfortably into the surface of the person's finger. In an example, the inner ring layer has a first magnitude of undulation when the solenoids or pistons are contracted and a second magnitude of undulation when the solenoids or pistons are extended, wherein the second magnitude is greater than the first magnitude. In an example, the inner ring layer is not undulating when the solenoids or pistons are contracted, but is undulating when the solenoids or pistons are extended. In an example, individual solenoids or pistons (or sets of solenoids or pistons) can be independently extended or contracted.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

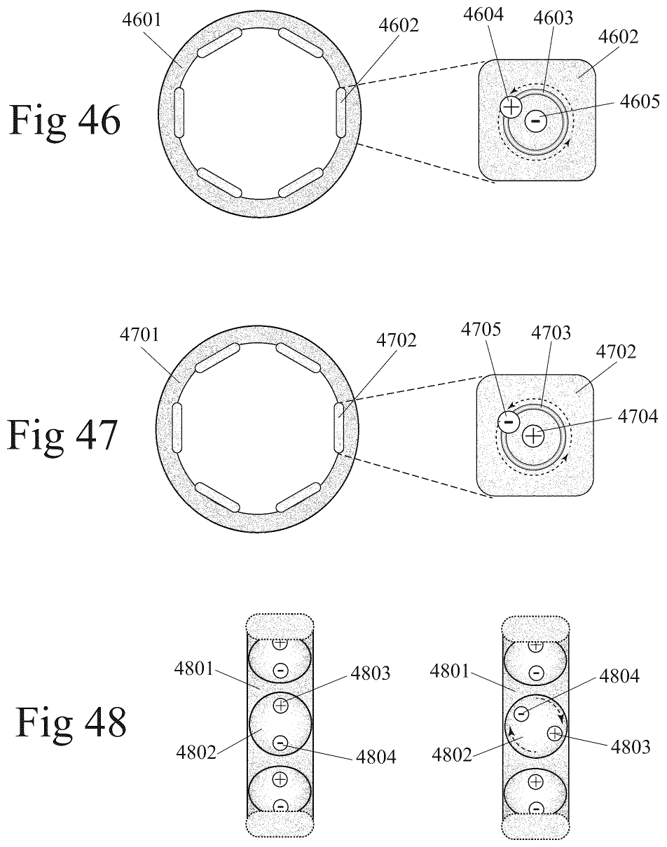

FIG. 46 shows a smart ring comprising: a finger ring 4601 which is worn on a person's finger; a plurality of optical sensor sets (including set 4602) on the ring; wherein each optical sensor set further comprises at least one light receiver (such as 4605), an arcuate (e.g. circular) track or channel (such as 4603), and at least one light emitter (such as 4604) which moves along the track or channel around the at least one light receiver; wherein light from a light emitter is directed toward the person's finger, wherein the light is received by a light receiver after the light has interacted with the person's finger (e.g. been reflected by and/or transmitted through the finger), and wherein biometric information (e.g. biometric parameter values) is obtained by analyzing changes (e.g. changes in spectral distribution and/or intensity) in the light caused by interaction between the light and the person's finger. The left portion of FIG. 46 shows a cross-sectional side view of this ring. The right portion of FIG. 46 shows an enlarged view of the finger-facing side of an optical sensor set on this ring.

In an example, a ring can have at least three optical sensor sets. In an example, a ring can have at least six optical sensor sets. In an example, optical sensor sets can be distributed around the entire circumference of the ring. In an example, optical sensor sets can collectively span at least half of the circumference of the ring. In an example, an optical sensor set can comprise a central light receiver, a circular track or channel around the light receiver, and a light emitter which moves around the light receiver along the track or channel. In an example, a ring can further comprise a plurality of actuators which move light emitters along tracks or channels.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 47 shows a smart ring comprising: a finger ring 4701 which is worn on a person's finger; a plurality of optical sensor sets (including set 4702) on the ring; wherein each optical sensor set further comprises at least one light emitter (such as 4704), an arcuate (e.g. circular) track or channel (such as 4703), and at least one light receiver (such as 4705) which moves along the track or channel around the at least one light receiver; wherein light from a light emitter is directed toward the person's finger, wherein the light is received by a light receiver after the light has interacted with the person's finger (e.g. been reflected by and/or transmitted through the finger), and wherein biometric information (e.g. biometric parameter values) is obtained by analyzing changes (e.g. changes in spectral distribution and/or intensity) in the light caused by interaction between the light and the person's finger. The left portion of FIG. 47 shows a cross-sectional side view of this ring. The right portion of FIG. 47 shows an enlarged view of the finger-facing side of an optical sensor set on this ring.

In an example, a ring can have at least three optical sensor sets. In an example, a ring can have at least six optical sensor sets. In an example, optical sensor sets can be distributed around the entire circumference of the ring. In an example, optical sensor sets can collectively span at least half of the circumference of the ring. In an example, an optical sensor set can comprise a central light emitter, a circular track or channel around the light emitter, and a light receiver which moves around the light emitter along the track or channel. In an example, a ring can further comprise a plurality of actuators which move light receivers along tracks or channels.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 48 shows two radial views, at two different times, of a section of the inner circumference of a smart ring comprising: a ring 4801 which is worn on a person's finger; a plurality of rotatable optical sensor sets (including set 4802) on the ring; wherein each optical sensor set further comprises at least one light emitter (such as 4803) and at least one light receiver (such as 4804); wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 48 shows this view at a first time when optical sensor sets are in a first configuration. The right portion of FIG. 48 shows this view at a second time when optical sensor sets are rotated into a second configuration.

In an example, a ring can have at least three optical sensor sets. In an example, a ring can have at least six optical sensor sets. In an example, optical sensor sets can be distributed around the entire circumference of the ring. In an example, optical sensor sets can collectively span at least half of the circumference of the ring. In an example, there can be one light emitter and one light receiver in each optical sensor set. In an example, there can be two light emitters and one light receiver in each optical sensor set. In an example, a light emitter and a light receiver can be diametrically-opposite each other in an optical sensor set. In an example, a light emitter can be in the center of an optical sensor set and a light receiver can be in a non-central location on the set. In an example, a light receiver can be in the center of an optical sensor set and a light emitter can be in a non-central location on the set. In an example, individual optical sensor sets can be independently rotated.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

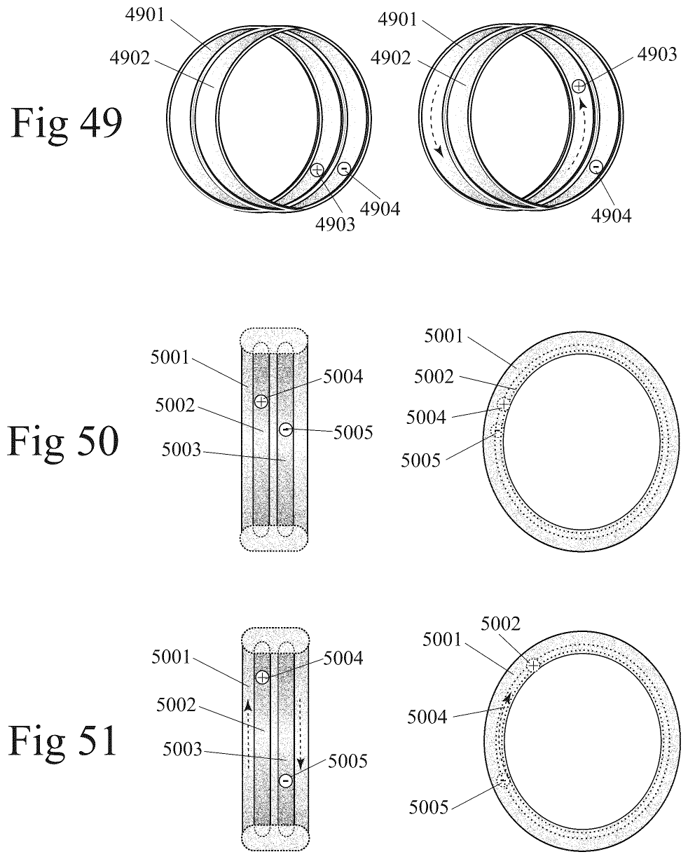

FIG. 49 shows two oblique views, at two different times, of a smart ring comprising: a first ring 4901 which is worn on a person's finger; a second ring 4902 which is worn on the person's finger; wherein the circumferences of the first ring and the second ring are parallel to each other, wherein the first ring and the second ring are rotatably-connected to each other, and wherein the first ring can be rotated relative to the second ring, or vice versa; at least one light emitter 4903 on the first ring; and at least one light receiver 4904 on the second ring; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 49 shows the first and second rings in a first configuration. The right portion of FIG. 49 shows the first and second rings having been rotated into a second configuration.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIGS. 50 and 51 show radial interior views and cross-sectional side views of a smart ring at two different times. This smart ring comprises: a ring 5001 which is worn on a person's finger; a first track (or channel) 5002 on the inner circumference of the ring; a second track (or channel) 5003 on the inner circumference of the ring, wherein the first track (or channel) and the second track (or channel) are parallel to each other; at least one light emitter 5004 which moves along the first track (or channel); and at least one light receiver 5005 which moves along the second track (or channel); wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portions of FIGS. 50 and 51 show radial interior views of a section of this ring at two different times. The right portions of FIGS. 50 and 51 show cross-sectional side views of this ring at two different times.

In an example, a first track (or channel) can span the entire inner circumference of the ring. In an example, a light emitter can travel around the entire inner circumference of a ring on a first track (or channel). In an example, a first track (or channel) can span a portion of the inner circumference of the ring. In an example, a first track (or channel) can span between 20% and 40% of the inner circumference of the ring. In an example, a ring can comprise a plurality of first tracks (or channels) which span different sections of the inner circumference of the ring. In an example, there can be a light emitter associated with each of these first tracks, moving along a first track. In an example, the ring can further comprise one or more actuators which move one of more light emitters along the one or more first tracks. In an example, a ring can include one or more actuators which move one or more light emitters along one or more first tracks (or channels).

In an example, a second track (or channel) can span the entire inner circumference of the ring. In an example, a light receiver can travel around the entire inner circumference of a ring on a second track (or channel). In an example, a second track (or channel) can span a portion of the inner circumference of the ring. In an example, a second track (or channel) can span between 20% and 40% of the inner circumference of the ring. In an example, a ring can comprise a plurality of second tracks (or channels) which span different sections of the inner circumference of the ring. In an example, there can be a light receiver associated with each of these second tracks, moving along a second track. In an example, the ring can further comprise one or more actuators which move one or more light receivers along the one or more second tracks.

In an variation on this example, a ring can comprise a first rotatable sub-ring (instead of a track) with a light emitter on it and a second rotatable sub-ring (instead of a track) with a light receiver on it. In an example, first and second rotatable sub-rings can be parallel to each other. In an example, first and second sub-rings can be rotated separately and independently. In an example, first and second sub-rings can be inside a main (outer) ring. In an example, a ring can further comprise one or more actuators which rotate first and/or second sub-rings. Moving the sub-rings separately and independently can change the locations of one or more light emitters and light receivers on the inner circumference of the ring, as well as the distance(s) between them. This enables optical scanning of different regions and depths of finger tissue.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

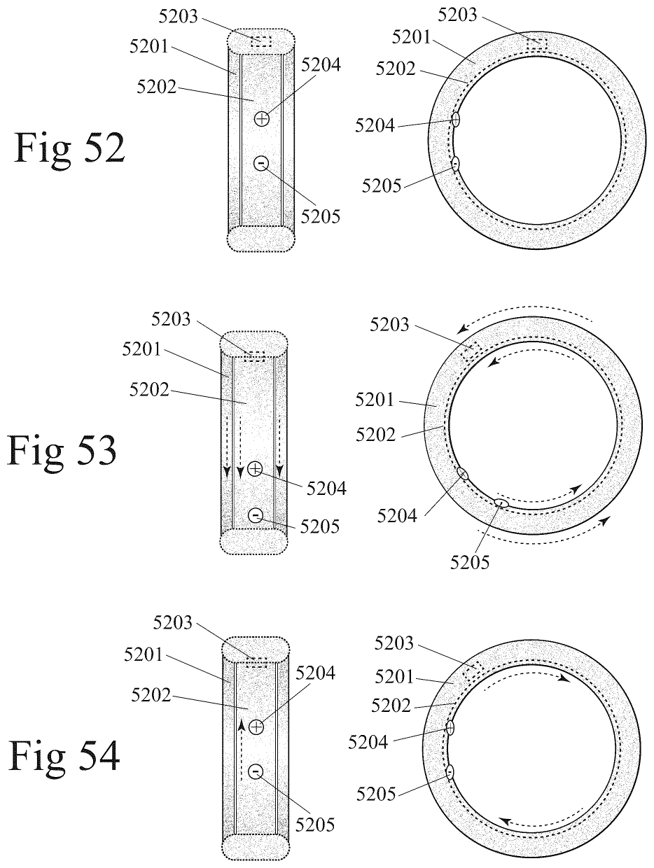

FIGS. 52 through 54 show radial interior views and cross-sectional side views of a smart ring at three different times. The left portions of FIGS. 52 through 54 show radial interior views of a section of this ring at three different times. The right portions of FIGS. 52 through 54 show cross-sectional side views of this ring at three different times.

This smart ring comprises: an outer ring 5201 which is worn on a person's finger; a rotatable inner ring 5202 which is worn on the person's finger, wherein the rotatable inner ring is narrower than the outer ring and/or closer to the surface of the person's finger than the outer ring; at least one light emitter 5204 on the rotatable inner ring; at least one light receiver 5205 on the rotatable inner ring, wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger; and a motion and/or orientation sensor 5203, wherein the rotatable inner ring is (automatically) rotated based on data from the motion and/or orientation sensor.

In an example, the ring can further comprise an actuator which automatically rotates the inner ring in response to changes in the location and/or orientation of the ring as detected by the motion and/or orientation sensor. In an example, the motion and/or orientation sensor can detect when the outer ring has been unintentionally rotated around a person's finger, which triggers an actuator to rotate the inner ring to counteract this unintentional rotation. This keeps the light emitters and receivers aligned with preferred locations on the circumference of the finger. In an example, unintentional rotation of the outer ring can move light emitters and receivers away from preferred locations on the circumference of the finger, but this unintentional rotation can be detected by the motion and/orientation sensor and an actuator can automatically counter-rotate the inner ring to keep the light emitters and receivers aligned with these preferred locations. In an example, the inner ring can be closer to the surface of the person's finger than the outer ring. In an example, the inner ring can be narrower than the outer ring. In an example, the inner ring can be at least partially within the outer ring.

FIG. 52 shows this smart ring at a first time before it has been unintentionally rotated, when the light emitter and the light receiver are on their preferred locations on the circumference of a person's finger. FIG. 53 shows this smart ring at a second time after both the outer ring and the inner ring have both been unintentionally rotated, causing the light emitter and the light receiver to shift away from their preferred locations on the circumference of the person's finger. FIG. 54 shows this smart ring at a third time after the unintentional rotation has been detected by the motion and/or orientation sensor, triggering the inner ring to be counter-rotated relative to the outer ring, thereby returning the light emitter and the light receiver to their preferred locations on the circumference of the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

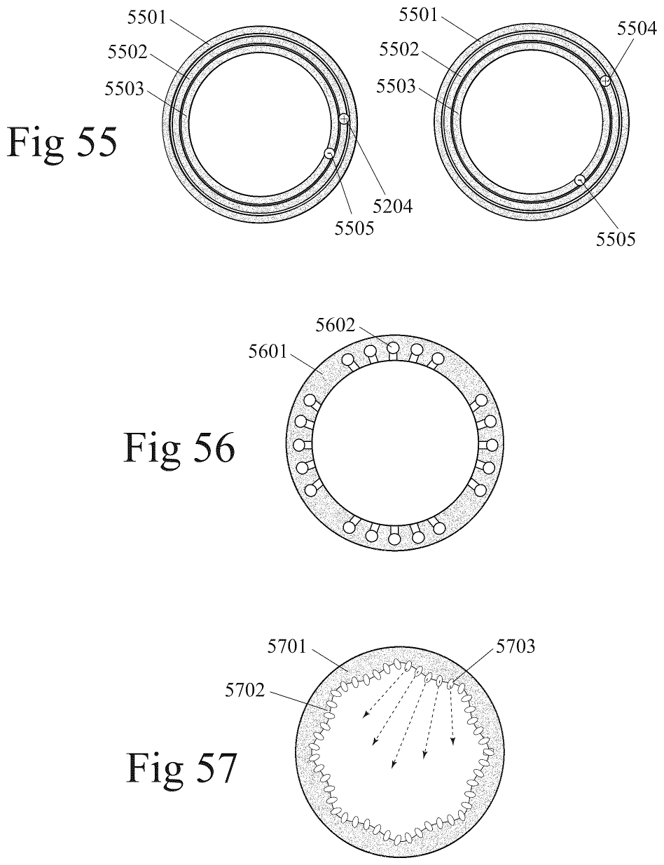

FIG. 55 shows two cross-sectional side views, at two different times, of a smart ring comprising: an outer ring 5501 which is worn on a person's finger at a first distance from the surface of the finger; a middle ring 5502 which is worn on the person's finger at a second distance from the surface of the finger, wherein the second distance is less than the first distance, and wherein the middle ring can be independently rotated (relative to other rings); an inner ring 5503 which is worn on the person's finger at a third distance from the surface of the finger, wherein the third distance is less than the second distance, and wherein the inner ring can be independently rotated (relative to other rings); at least one light emitter 5504 on a first ring selected from the middle ring or the inner ring; and at least one light emitter 5505 on a second ring selected from the middle ring or the inner ring, wherein the second ring is different than the first ring; wherein light from a light emitter is directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 55 shows the middle and inner rings in a first rotational configuration. The right portion of FIG. 55 shows the middle and inner rings in a second rotational configuration.

In an example, the outer, middle, and inner rings can be concentric. In an example, the outer, middle, and inner rings can be nested. In an example, at least one light emitter can be on the middle ring and at least one light receiver can be on the inner ring. In another example, at least one light emitter can be on the inner ring and at least one light receiver can be on the middle ring. In an example, the middle ring can be transparent. In an example, rotating the middle ring and/or inner ring changes the locations of at least one light emitter and at least one light receiver. In an example, rotating the middle ring and/or inner ring changes the distance between a light emitter and a light receiver.

In an example, the ring can further comprise one or more actuators which rotate the middle and/or inner rings relative to other rings. In an example, the ring can further comprise a first actuator which rotates the middle ring relative to the outer ring and a second actuator which rotates the inner ring relative to the outer ring. In an example, one side (e.g. the side facing away from the finger) of the middle ring and/or one side (e.g. the side facing away from the finger) of the inner ring can have cogs, teeth, notches, threads, or indentations which are engaged by one or more actuators to rotate the middle ring and/or the inner ring.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 56 shows a cross-sectional side view of a smart ring comprising: a finger ring 5601 which is worn on a person's finger; a plurality of optical elements (e.g. light emitters and/or light receivers) on the ring including optical element 5602, wherein light pathways (e.g. light beam vectors) between different optical elements and the inner circumference of the ring intersect the inner circumference of the ring and/or the surface of the person's finger at different angles.

FIG. 56 can also be described as showing a cross-sectional side view of a smart ring comprising: a finger ring 5601 which is worn on a person's finger; a plurality of optical sensor sets on the ring, wherein each optical sensor set includes a plurality of light emitters (such as 5602) and/or light receivers, and wherein light pathways (e.g. light beam vectors) between different light emitters within a set and the inner circumference of the ring intersect the inner circumference of the ring and/or the surface of the person's finger at different angles. In an example, all optical sensor sets can include both light emitters and light receivers. In an example, some optical sensor sets can include only light emitters and some optical sensor sets can include only light receivers.

In an example, light pathways between optical elements (e.g. light emitters and/or light receivers) and the inner circumference of the ring and/or the surface of the person's finger can be directed by light guides, prisms, waveguides, optical fibers, or mirrors. In an example, the ring can further comprise a plurality of light guides, prisms, waveguides, optical fibers, or mirrors which direct the transmission of light between optical elements (e.g. light emitters and/or light receivers) and the inner circumference of the ring and/or the surface of the person's finger (or vice versa). In an example, light guides, prisms, waveguides, optical fibers, or mirrors can direct light from different optical elements (e.g. light emitters and/or light receivers) in a set along different vectors which intersect the inner circumference of the ring and/or the surface of the finger at different angles. In an example, light guides, prisms, waveguides, optical fibers, or mirrors can transmit light from different light emitters in a toward the finger along different vectors and/or angles. In an example, light guides, prisms, waveguides, optical fibers, or mirrors can transmit reflected or transmitted light from the finger toward different light receivers in a set along different vectors and/or angles.

In an example, the intersection angles at which light pathways between optical elements (e.g. light emitters or light receivers) in a set intersect the inner circumference of the ring and/or the surface of a finger can vary by location within a set. In an example, the intersection angles at which light pathways between optical elements (e.g. light emitters or light receivers) in a set intersect the inner circumference of the ring and/or the surface of a finger can vary as a function of distance from the center of a set. In an example, the intersection angles at which light pathways between optical elements (e.g. light emitters or light receivers) in a set intersect the inner circumference of the ring and/or the surface of a finger can increase as a function of distance from the center of a set. In an example, the intersection angles at which light pathways between optical elements (e.g. light emitters or light receivers) in a set intersect the inner circumference of the ring and/or the surface of a finger can decrease as a function of distance from the center of a set. In an example, the intersection angles at which light pathways between optical elements (e.g. light emitters or light receivers) in a set intersect the inner circumference of the ring and/or the surface of a finger can vary as a function of distance from the end of a set.

In an example, light pathways (e.g. light beam vectors) between central optical elements (e.g. light emitters or light receivers at the center of a set) and the inner circumference of a ring intersect the inner circumference at 90-degree angles (e.g. perpendicularly), but light pathways (e.g. light beam vectors) between non-central optical elements (e.g. light emitters or light receivers which are not at the center of the set) and the inner circumference of the ring intersect the inner circumference of the ring at acute angles (e.g. not perpendicularly). In an example, light pathways (e.g. light beam vectors) between central optical elements (e.g. light emitters or light receivers at the center of a set) and the inner circumference of a ring intersect the inner circumference at 90-degree angles (e.g. perpendicularly), but light pathways (e.g. light beam vectors) between non-central optical elements (e.g. light emitters or light receivers which are not at the center of the set) and the inner circumference of the ring intersect the inner circumference of the ring at obtuse angles (e.g. not perpendicularly).

In an example, light from a light emitter can be directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 57 shows a cross-sectional side view of a smart ring comprising: a finger ring 5701 which is worn on a person's finger, wherein the inner surface of the finger ring (e.g. the side of the ring which faces radially-inward toward the person's finger) has undulations such as 5702; and a plurality of optical elements (e.g. light emitters and/or light receivers) such as 5703 on the undulations.

In an example, light emitters and/or light receivers can be located on selected waves, phases, or sections of an undulating inner surface (e.g. radially-inward side) of a ring. In an example, light emitters and/or light receivers can be located on the waves, phases, and/or sections of an undulating inner surface (e.g. radially-inwards side) of a ring which protrude toward the person's finger. In an example, light emitters can be substantially perpendicular to an undulating inner surface of the finger ring so that vectors of light beams from different light emitters intersect the surface of the person's finger at different angles. In an example, light receivers can be substantially perpendicular to an undulating inner surface of the finger ring so that light beams exiting the finger are transmitted to different light receivers at different angles. In an example, undulations can be sinusoidal undulations.

In an example, light from a light emitter can be directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

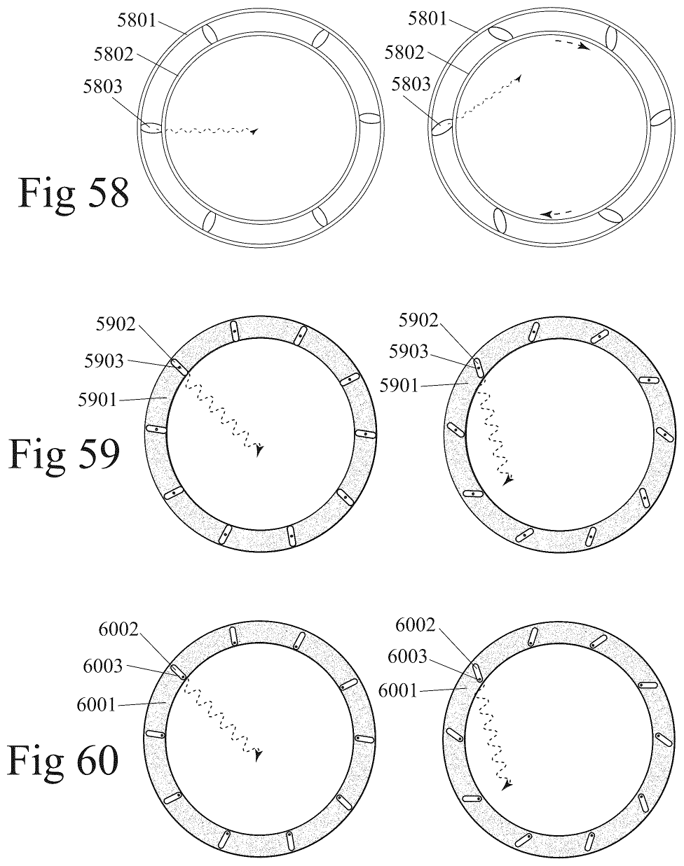

FIG. 58 shows two cross-sectional side views, at two different times, of a smart ring comprising: an outer ring 5801 which is worn on a person's finger at a first distance from the surface of the finger; an inner ring 5802 which is worn on the person's finger at a second distance from the surface of the finger, wherein the second distance is less than the first distance; and a plurality of light emitters (including 5803) which are movably-connected to the outer ring and to the inner ring, wherein movement (e.g. rotation) of the inner ring relative to the outer ring, or vice versa, changes the angles (e.g. vectors) at which light beams from the plurality of light emitters enter the surface of the person's finger.

The left portion of FIG. 58 shows the outer ring and the inner ring at a first time in a first configuration which causes the light emitters to emit light into the surface of the person's finger at a first set of angles (e.g. 90-degree angles). The right portion of FIG. 58 shows the outer ring and the inner ring at a second time in a second configuration which causes the light emitters to emit light into the surface of the person's finger at a second set of angles (e.g. acute angles).

In an example, the outer and inner rings can be concentric. In an example, the outer and inner rings can be nested. In an example, the overall ring can further comprise one or more actuators which move (e.g. rotate) the inner ring relative to the outer ring, or vice versa. In an example, one side of the inner ring or one side of the outer ring can have cogs, teeth, notches, threads, or indentations which are engaged by one or more actuators to move (e.g. rotate) the inner ring or the outer ring. In an example, a first end of a light emitter can be movably-connected (e.g. with a joint or hinge) to the outer ring and a second end of the light emitter can be movably-connected (e.g. with a joint or hinge) to the inner ring. In an example, moving (e.g. rotating) the outer ring or the inner ring tilts and/or pivots the light emitter, thereby changing the angle at which light beams from the light emitter enter the person's finger.

In an example, the ring can further comprise a plurality of light receivers. In an example, light from a light emitter can be directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 59 shows two cross-sectional side views, at two different times, of a smart ring comprising: an outer ring 5901 which is worn on a person's finger at a first distance from the surface of the finger; an inner ring 5902 which is worn on the person's finger at a second distance from the surface of the finger, wherein the second distance is less than the first distance; and a plurality of light emitters (including 5903) which are movably-connected to the outer ring and to the inner ring, wherein movement (e.g. rotation) of the inner ring relative to the outer ring (or vice versa) rotates the light emitters around central axes of the light emitters, thereby changing the angles (e.g. vectors) at which light beams from the light emitters enter the surface of the person's finger.

The left portion of FIG. 59 shows the outer ring and the inner ring at a first time in a first configuration which causes the light emitters to emit light into the surface of the person's finger at a first set of angles (e.g. 90-degree angles). The right portion of FIG. 59 shows the outer ring and the inner ring at a second time in a second configuration which causes the light emitters to emit light into the surface of the person's finger at a second set of angles (e.g. acute angles).

In an example, the outer and inner rings can be concentric. In an example, the outer and inner rings can be nested. In an example, the overall ring can further comprise one or more actuators which move (e.g. rotate) the inner ring relative to the outer ring, or vice versa. In an example, one side of the inner ring or one side of the outer ring can have cogs, teeth, notches, threads, or indentations which are engaged by one or more actuators to move (e.g. rotate) the inner ring or the outer ring. In an example, the ring can further comprise a plurality of light receivers. In an example, light from a light emitter can be directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 60 shows two cross-sectional side views, at two different times, of a smart ring comprising: an outer ring 6001 which is worn on a person's finger at a first distance from the surface of the finger; an inner ring 6002 which is worn on the person's finger at a second distance from the surface of the finger, wherein the second distance is less than the first distance; and a plurality of light emitters (including 6003) which are movably-connected to the outer ring and to the inner ring, wherein movement (e.g. rotation) of the inner ring relative to the outer ring (or vice versa) tilts and/or pivots the light emitters around their ends, thereby changing the angles (e.g. vectors) at which light beams from the light emitters enter the surface of the person's finger.

The left portion of FIG. 60 shows the outer ring and the inner ring at a first time in a first configuration which causes the light emitters to emit light into the surface of the person's finger at a first set of angles (e.g. 90-degree angles). The right portion of FIG. 60 shows the outer ring and the inner ring at a second time in a second configuration which causes the light emitters to emit light into the surface of the person's finger at a second set of angles (e.g. acute angles).

In an example, the outer and inner rings can be concentric. In an example, the outer and inner rings can be nested. In an example, the overall ring can further comprise one or more actuators which move (e.g. rotate) the inner ring relative to the outer ring, or vice versa. In an example, one side of the inner ring or one side of the outer ring can have cogs, teeth, notches, threads, or indentations which are engaged by one or more actuators to move (e.g. rotate) the inner ring or the outer ring. In an example, the ring can further comprise a plurality of light receivers. In an example, light from a light emitter can be directed toward the person's finger, wherein the light is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

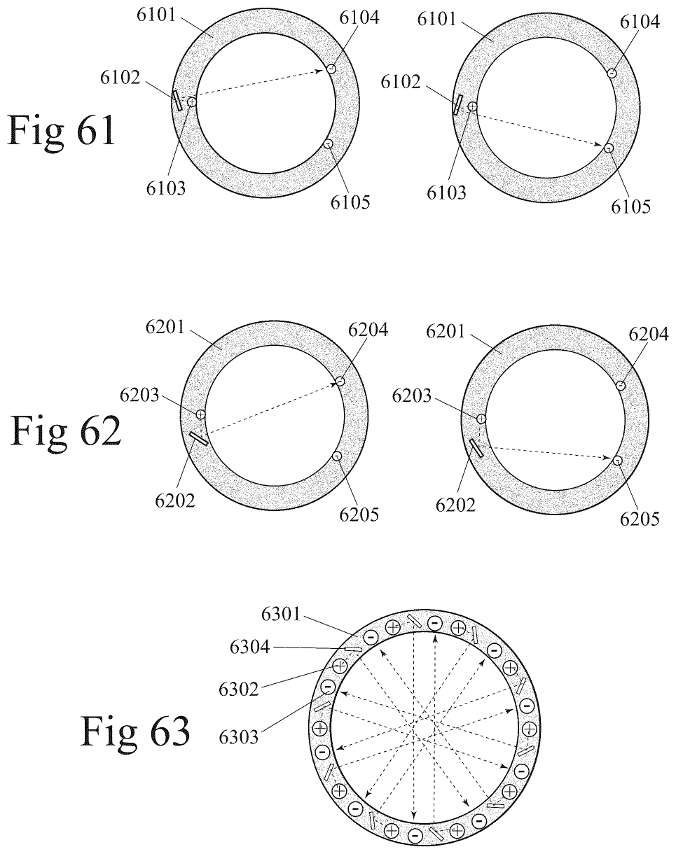

FIG. 61 shows two cross-sectional side views, at two different times, of a smart ring comprising: a ring 6101 which is worn on a person's finger; at least one light emitter 6103 on the ring; at least one movable (e.g. rotatable, pivotable, and/or tiltable) reflective component (e.g. mirror) 6102 on the ring, wherein the reflective component is farther from the inner circumference of the ring (and the surface of the person's finger) than the light emitter, wherein light from the light emitter is reflected by the reflective component toward the person's finger, and wherein moving (e.g. rotating, pivoting, and/or tilting) the reflective component changes the angle and/or location at which light from the light emitter enters the person's finger; and at least one light receiver 6104 and 6105 on the ring, wherein light from a light emitter is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

The left portion of FIG. 61 shows the ring at a first time in a first configuration when the reflective component directs light from the light emitter toward the person's finger at a first angle. The right portion of FIG. 61 shows the ring at a second time in a second configuration when the reflective component has been moved (e.g. rotated, pivoted, and/or tilted) and now directs light from the light emitter toward the person's finger at a second angle.

In an example, a ring can further comprise an actuator which moves (e.g. rotates, pivots, and/or tilts) a reflective component (e.g. mirror). In an example, the actuator can be an electromagnetic motor. In an example, the reflective component can be a digital micromirror device. In an example, a ring can comprise a plurality of actuators which move (e.g. rotate, pivot, and/or tilt) a plurality of reflective components which redirect light beams from a plurality of light emitters. In an example, a ring can comprise a plurality of light emitters and plurality of movable reflective components, wherein the reflectively components can be individually and independently moved.

In an example, moving (e.g. rotating, pivoting, and/or tilting) a reflective component (e.g. mirror) can cause light beams from a light emitter to scan through different locations and/or depths of finger tissue. In an example, moving (e.g. rotating, pivoting, and/or tilting) a reflective component (e.g. mirror) can cause light beams from a light emitter to travel along different optical pathways (e.g. from the light emitter to different light receivers). In an example, the center of the light emitter can be a first distance from the surface of the finger, the center of the reflective component can be a second distance from the surface of the finger, and the second distance can be greater than the first distance.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 62 shows two cross-sectional side views, at two different times, of a smart ring comprising: a ring 6201 which is worn on a person's finger; at least one light emitter 6203 on the ring; at least one movable (e.g. rotatable, pivotable, and/or tiltable) reflective component (e.g. mirror) 6202 on the ring wherein light from the light emitter is reflected by the reflective component toward the person's finger, and wherein moving (e.g. rotating, pivoting, and/or tilting) the reflective component changes the angle and/or location at which light from the light emitter enters the person's finger; and at least one light receiver 6204 and 6205 on the ring, wherein light from a light emitter is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

The left portion of FIG. 62 shows the ring at a first time in a first configuration when the reflective component directs light from the light emitter toward the person's finger at a first angle. The right portion of FIG. 62 shows the ring at a second time in a second configuration when the reflective component has been moved (e.g. rotated, pivoted, and/or tilted) and now directs light from the light emitter toward the person's finger at a second angle.

In an example, a ring can further comprise an actuator which moves (e.g. rotates, pivots, and/or tilts) a reflective component (e.g. mirror). In an example, the actuator can be an electromagnetic motor. In an example, the reflective component can be a digital micromirror device. In an example, a ring can comprise a plurality of actuators which move (e.g. rotate, pivot, and/or tilt) a plurality of reflective components which redirect light beams from a plurality of light emitters. In an example, a ring can comprise a plurality of light emitters and plurality of movable reflective components, wherein the reflectively components can be individually and independently moved.

In an example, moving (e.g. rotating, pivoting, and/or tilting) a reflective component (e.g. mirror) can cause light beams from a light emitter to scan through different locations and/or depths of finger tissue. In an example, moving (e.g. rotating, pivoting, and/or tilting) a reflective component (e.g. mirror) can cause light beams from a light emitter to travel along different optical pathways (e.g. from the light emitter to different light receivers).

In an example, a reflective component can be to one side of the light emitter whose light it redirects. In an example, a reflective component can be to the right or to the left of a light emitter on the dorsal portion (e.g. quadrant) or a ventral portion (e.g. quadrant) of the ring. In an example, a reflective component can be on the dorsal side or on the ventral side a light emitter on a left portion (e.g. quadrant) or a right portion (e.g. quadrant) of the ring.

In this example, a reflective component is on a different location on the circumference of the ring than the light emitter whose light it redirects. In an example, a light emitter can be at a first location on the circumference of the ring and a reflective component which redirects light from that light emitter can be a second location on the circumference of the ring, wherein the two locations differ by 1 to 10 degrees (out of the 360 degrees around the entire circumference of the ring). In an example, a reflective component can be 1 to 10 degrees in a clockwise or counter-clockwise direction (around the ring) from a light emitter whose light it redirects.

In an example, a reflective component can be substantially the same distance from the inner circumference of the ring as the light emitter whose light it redirects. In an example, the center of the light emitter can be a first distance from the surface of the finger, the center of the reflective component can be a second distance from the surface of the finger, and the second distance is greater than 80% of the first distance and less than 120% of the first distance.

In an example, a reflective component can be moved (e.g. rotated, pivoted, and/or tilted) by exposure to an electromagnetic field. In an example, a reflective component can be moved (e.g. rotated, pivoted, and/or tilted) by changing an electromagnetic field to which the reflective component is exposed. In an example, a reflective component can be made, in part, by a material (e.g. magnetic material) which responds to an electromagnetic field, wherein the reflective component is moved (e.g. rotated, pivoted, and/or tilted) by change in the electromagnetic field. In an example, a reflective component can be moved by changes in the direction and/or magnitude of an electromagnetic field to which it is exposed.

In an example, a reflective component can have a central longitudinal axis around which is it rotated, pivoted, and/or tilted to redirect light from a light emitter. In an example, a reflective component can have a central lateral axis around which is it rotated, pivoted, and/or tilted to redirect light from a light emitter. In an example, a reflective component can have vertexes around which is it rotated, pivoted, and/or tilted to redirect light from a light emitter. In an example a reflective component can be suspended in an electromagnetic field, within which it is rotated, pivoted, and/or tilted. In an example, a series or sequence of movable micromirrors can redirect light from a light emitter toward a person's finger. In an example, an arcuate array of movable micromirrors can redirect light from a light emitter toward a person's finger.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 63 shows a cross-sectional side view of a smart ring comprising: a ring 6301 which is worn on a person's finger; a plurality of light emitters (including 6302) on the ring; a plurality of light receivers (including 6303) on the ring; a plurality of reflective components (including 6304) on the ring; wherein light from a light emitter is reflected by a reflective component toward the person's finger, wherein light from the light emitter is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, a reflective component can be a mirror. In an example, a reflective component can be a light guide and/or waveguide. In an example, a reflective component can be moved (e.g. rotated, pivoted, and/or tilted) to change the vector of light beams reflected from a light emitter. In an example, a reflective component can be moved (e.g. rotated, pivoted, and/or tilted) to change the angle and/or location at which light beams reflected from a light emitter enter the person's finger. In an example, a reflective component can be moved (e.g. rotated, pivoted, and/or tilted) to scan different regions and/or depths of finger tissue. In an example, a ring can further comprise one or more actuators which move (e.g. rotate, pivot, and/or tilt) one or more reflective components. In an example, a ring can comprise a plurality of light emitters and plurality of movable reflective components, wherein the reflectively components can be individually and independently moved.

In an example, a ring can comprise a repeating sequence and/or series of light emitters, reflective components, and light emitters. In an example, a ring can comprise a repeating sequence and/or series of light emitters, reflective components, and light emitters around the circumference of the ring. In an example, a ring can comprise a plurality of optical sensor sets on the inner circumference of the ring, wherein each set further comprises a light emitter, a reflective component which reflects light from the light receiver, and a light receiver. In an example, a ring can comprise a plurality of optical sensor sets on the inner circumference of the ring, wherein each set further comprises two or more light emitters, a reflective component which reflects light from the two or more light receivers, and at least one light receiver.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

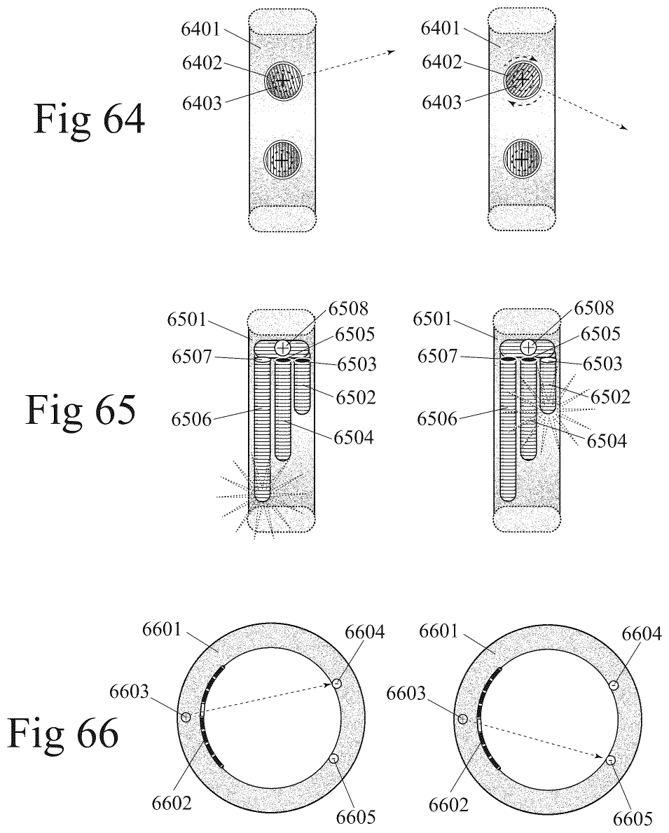

FIG. 64 shows two radial views, at two different times, of the inner circumference of a smart ring comprising: ring 6401 which is worn on a person's finger; a plurality of light emitters (including 6402) on the ring; and a plurality of movable (e.g. rotatable, pivotable, and/or tiltable) refractive components (including 6403) which change the directions of light beams from the light emitters when the refractive components are moved. In an example, the ring can further comprise a plurality of light receivers, wherein light from a light emitter is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger. The left portion of FIG. 64 shows this view at a first time when a refractive component directs light from a light emitter in a first direction. The right portion of FIG. 64 shows this view at a second time when the refractive component has been rotated and now directs light from the light emitter in a second direction.

In an example, a refractive component can be a lens, prism, or waveguide. In an example, a refractive component can be a rotatable lens, prism, or waveguide. In an example, a refractive component can be a pivotable and/or tiltable lens, prism, or waveguide. In an example, a refractive component can be a variable-focus lens. In an example, a refractive component can be a tunable lens. In an example, a refractive component can be a Fresnel lens or portion of a Fresnel lens. In an example, a refractive component can have an optical grating. In an example, a ring can further comprise one or more actuators which move (e.g. rotate, pivot, and/or tilt) one or more refractive components. In an example, a plurality of refractive components can be individually and independently moved, enabling individual and independent control over the directions of light beams from a plurality of light emitters. In an example, this enables a ring to scan different regions and depths of finger tissue.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 65 shows two radial views, at two different times, of the inner circumference of a smart ring comprising: a ring 6501 which is worn on a person's finger; at least one light emitter 6508 on the ring; a plurality of light guides and/or optical fibers (including 6502, 6504, and 6506) on the ring which transmit light from a light emitter to different locations on the interior circumference of the ring, wherein light from the light emitter is emitted from these different locations toward the person's finger; and a plurality of light valves (including 6503, 6505, and 6507) on the ring which are selectively adjusted to allow or block the transmission of light from the light emitter through one or more selected light guides and/or optical fibers. The left portion of FIG. 65 shows this view at a first time when only light valve 6507 is open, allowing light from the light emitter to be transmitted only through light guide and/or optical fiber 6506. The right portion of FIG. 65 shows this view at a second time when only light valve 6503 is open, allowing light from the light emitter to be transmitted only through light guide and/or optical fiber 6502.

In an example, the ring can further comprise at least one light receiver, wherein light from a light emitter is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

In an example, a light guide and/or optical fiber can be a fiber optic tube and/or cable. In an example, a light guide and/or optical fiber can be a waveguide. In an example, a light guide and/or optical fiber can transmit light through internal reflection. In an example, a light guide and/or optical fiber can transmit light via total internal reflection. In an example, different light guides and/or optical fibers on the ring can have different lengths, thereby transmitting light to different locations on the inner circumference of the ring from which light is emitted toward the person's finger. In an example, light from a light emitter can be transmitted through different light guides and/or optical fibers at different times, enabling scanning different regions and/or depths of finder tissue from the same light emitter. In an example, (a subset of) a plurality of light guides and/or optical fibers can be parallel to each other. In an example, light guides and/or optical fibers can be arcuate.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 66 shows two cross-sectional side views, at two different times, of a smart ring comprising: a ring 6601 which is worn on a person's finger; at least one light emitter 6603 on the ring; a plurality of light valves (including 6602) at different locations on the ring, wherein a light valve has a first configuration which allows light from a light emitter to be transmitted (through the light valve) and a second configuration which does not allow light from the light emitter to be transmitted (through the light valve), and wherein a selected subset of one or more light valves can be selectively set in their first configurations and the rest of the light valves can be set in their second configurations to send light from a light emitter along a selected vector (e.g. direction and/or angle) to the person's finger; and at least one light receiver 6604 and 6605 on the ring, wherein light from a light emitter is received by a light receiver after the light has interacted with the person's finger (e.g. been reflected by and/or transmitted through the finger); and wherein biometric information (e.g. biometric parameter values) is obtained by analyzing changes (e.g. changes in spectral distribution and/or intensity) in the light caused by interaction between the light and the person's finger.

The left portion of FIG. 66 shows the ring at a first time when a first light valve is configured to transmit light from a light emitter toward the person's finger along a first vector (e.g. direction and/or angle). The right portion of FIG. 66 shows the ring at a second time when a second light valve is configured to transmit light from a light emitter toward the person's finger along a second vector (e.g. direction and/or angle).

In an example, a light valve can be a liquid crystal display. In an example, a light valve can be a movable (e.g. rotatable, pivotable, and/or tiltable) reflective component (e.g. mirror). In an example, a light valve can be a digital micromirror device. In an example, a light valve can comprise one or more light polarizing lenses. In an example, light valves can be located along the inner circumference of the ring. In an example, a ring can further comprise an actuator which changes the configuration of one or more light valves. In an example, a ring can further comprise an actuator which changes the configuration of one or more light valves by moving (e.g. rotating, pivoting, and/or tilting) one or more light valves. In an example, a light valve can be changed from a first configuration to a second configuration, or vice versa, by application (e.g. transmission) of electrical energy to (e.g. through) the light valve.

In an example, a ring can comprise an arcuate array of light valves. In an example, selective configuration of one light valve in an array of light valves into the first (e.g. open) configuration and the rest of the light valves into the second (e.g. blocked) configuration can selectively direct light beams emitted from a light emitter to a person's finger along a selected vector. In an example, selectively changing different light valves in an array of light valves into their first (e.g. open) configurations can change the vectors (e.g. directions and/or angles) of light beams from a light emitter entering a person's finger. In an example, sequentially changing different light valves in an array of light valves into their first (e.g. open) configurations can change the vectors (e.g. directions and/or angles) of light beams from a light emitter to scan different regions and/or depths of finger tissue.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

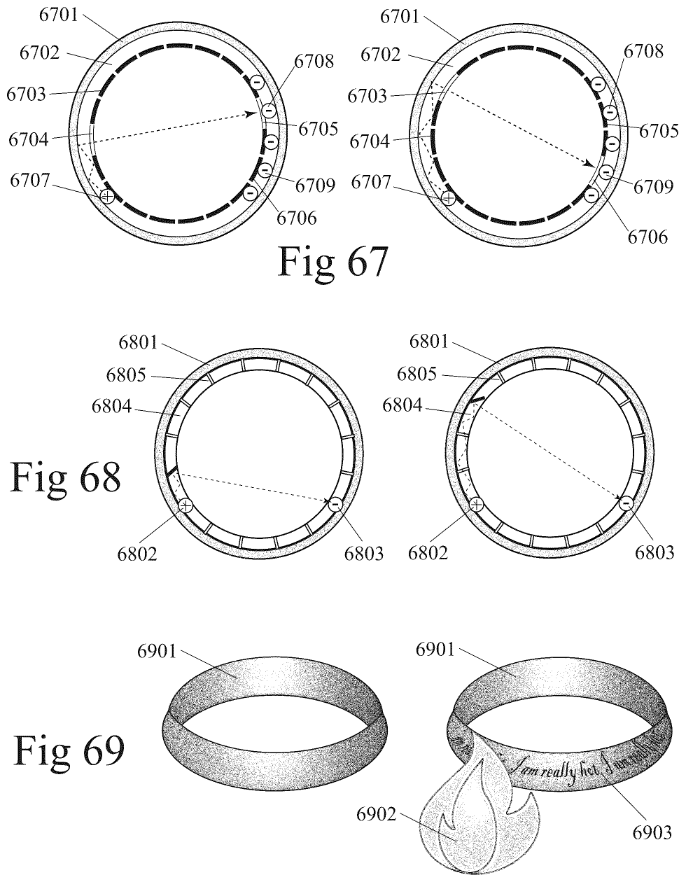

FIG. 67 shows two cross-sectional side views, at two different times, of a smart ring comprising: an outer ring 6701 which is worn on a person's finger at a first distance from the surface of the finger; an inner arcuate (e.g. circular) light-transmitting pathway 6702 at a second distance from the surface of the finger, wherein the second distance is less than the first distance; at least one light emitter 6707 on the ring; at least one light receiver 6708 and 6709 on the ring; and a plurality of light valves 6703 and 6704 between the arcuate light-transmitting pathway and the inner circumference of the ring, wherein a light valve has a first configuration which allows light to be transmitted (through the light valve) and a second configuration which does not allow light to be transmitted (through the light valve); wherein light from a light emitter is transmitted through the light-transmitting pathway, then transmitted through one or more selected light valves in the first configuration, then transmitted through (and/or reflected by) finger tissue, and then received by one or more light receivers; and wherein biometric information (e.g. biometric parameter values) is obtained by analyzing changes (e.g. changes in spectral distribution and/or intensity) in the light caused by interaction between the light and finger tissue. In this example, the ring further comprises a plurality of light valves 6705 and 6706 between the inner circumference of the ring and one or more light receivers.

The left portion of FIG. 67 shows the ring at a first time when a first light valve 6704 is configured to transmit light from the light-transmitting pathway toward the person's finger at a first location and this light is received by light receiver 6708. The right portion of FIG. 67 shows the ring at a second time when a second light valve 6703 is configured to transmit light from a light emitter toward the person's finger at a second location and this light is received by light receiver 6709.

In an example, a light-transmitting pathway can transmit light via total internal reflection. In an example, a light-transmitting pathway can be a waveguide. In an example, a light-transmitting pathway can be an optical fiber. In an example, a light-transmitting pathway can be made with metamaterial. In an example, a light-transmitting pathway can have a convex shape. In an example, a light-transmitting pathway can have a circular, ring, and/or annular shape. In an example, a ring can comprise a plurality arcuate light-transmitting pathways, wherein each pathway is a section (e.g. arc) of a circle or ring. In an example, there can be a separate light-transmitting pathway for each light emitter in a ring. In an example, two or more light emitters can transmit light through the same light-transmitting pathway.

In an example, a light valve can be a liquid crystal display. In an example, a light valve can be a movable (e.g. rotatable, pivotable, and/or tiltable) reflective component (e.g. mirror). In an example, a light valve can be a digital micromirror device. In an example, a light valve can comprise one or more light polarizing lenses. In an example, light valves can be located along the inner circumference of the ring. In an example, a ring can further comprise an actuator which changes the configuration of one or more light valves. In an example, a ring can further comprise an actuator which changes the configuration of one or more light valves by moving (e.g. rotating, pivoting, and/or tilting) one or more light valves. In an example, a light valve can be changed from a first configuration to a second configuration, or vice versa, by application (e.g. transmission) of electrical energy to (e.g. through) the light valve.

In an example, a ring can comprise an arcuate array of light valves. In an example, selective configuration of one light valve in an array of light valves into the first (e.g. open) configuration and the rest of the light valves into the second (e.g. blocked) configuration can selectively direct light beams to a person's finger from a selected location. In an example, selectively changing different light valves in an array of light valves into their first (e.g. open) configurations can change the vectors (e.g. directions and/or angles) of light beams from a light emitter entering a person's finger. In an example, sequentially changing different light valves in an array of light valves into their first (e.g. open) configurations can change the vectors (e.g. directions and/or angles) of light beams to scan different regions and/or depths of finger tissue.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 68 shows two cross-sectional side views, at two different times, of a smart ring comprising: an outer ring 6801 which is worn on a person's finger at a first distance from the surface of the finger; an inner arcuate (e.g. circular) light-transmitting pathway 6804 at a second distance from the surface of the finger, wherein the second distance is less than the first distance; at least one light emitter 6802 on the ring; at least one light receiver 6803 on the ring; and a plurality of light valves (including 6805) within the light-transmitting pathway; wherein a light valve has a first configuration which redirects light out from the light-transmission pathway to the person's finger and a second configuration allows light to continue transmission within the light-transmission pathway; wherein light from a light emitter is transmitted through the light-transmitting pathway, then redirected by a light valve toward the person's finger, then transmitted through (and/or reflected by) finger tissue, and then received by one or more light receivers; and wherein biometric information (e.g. biometric parameter values) is obtained by analyzing changes (e.g. changes in spectral distribution and/or intensity) in the light caused by interaction between the light and finger tissue.

The left portion of FIG. 68 shows the ring at a first time when a first light valve is configured to redirect light out from the light-transmitting pathway at a first location. The right portion of FIG. 68 shows the ring at a second time when a second light valve is configured to redirect light out from the light-transmitting pathway at a second location.

In an example, a light-transmitting pathway can transmit light via total internal reflection. In an example, a light-transmitting pathway can be a waveguide. In an example, a light-transmitting pathway can be an optical fiber. In an example, a light-transmitting pathway can be made with metamaterial. In an example, a light-transmitting pathway can have a convex shape. In an example, a light-transmitting pathway can have a circular, ring, and/or annular shape. In an example, a ring can comprise a plurality arcuate light-transmitting pathways, wherein each pathway is a section (e.g. arc) of a circle or ring. In an example, there can be a separate light-transmitting pathway for each light emitter in a ring. In an example, two or more light emitters can transmit light through the same light-transmitting pathway.

In an example, a light valve can be a liquid crystal display. In an example, a light valve can be a movable (e.g. rotatable, pivotable, and/or tiltable) reflective component (e.g. mirror). In an example, a light valve can be a digital micromirror device. In an example, a light valve can comprise one or more light polarizing lenses. In an example, light valves can be located along the inner circumference of the ring. In an example, a ring can further comprise an actuator which changes the configuration of one or more light valves. In an example, a ring can further comprise an actuator which changes the configuration of one or more light valves by moving (e.g. rotating, pivoting, and/or tilting) one or more light valves. In an example, a light valve can be changed from a first configuration to a second configuration, or vice versa, by application (e.g. transmission) of electrical energy to (e.g. through) the light valve.

In an example, a ring can comprise an arcuate array of light valves. In an example, selective configuration of one light valve in an array of light valves into the first (e.g. open) configuration and the rest of the light valves into the second (e.g. blocked) configuration can selectively direct light beams to a person's finger from a selected location. In an example, selectively changing different light valves in an array of light valves into their first (e.g. open) configurations can change the vectors (e.g. directions and/or angles) of light beams from a light emitter entering a person's finger. In an example, sequentially changing different light valves in an array of light valves into their first (e.g. open) configurations can change the vectors (e.g. directions and/or angles) of light beams to scan different regions and/or depths of finger tissue.

In an example, the ring can further comprise a battery. In an example, the ring can further comprise a power generator (or transducer) which generates (or transduces) electrical power from kinetic energy, thermal energy, ambient light energy, and/or ambient electromagnetic energy. In an example, the ring can further comprise a data processor wherein data from light receivers is analyzed. In an example, the ring can further comprise a data transmitter and/or receiver. In an example, the ring can further comprise a motion sensor. In an example, the ring can further comprise a pressure and/or contact sensor. In an example, the ring can further comprise a camera. Relevant variations discussed elsewhere in this disclosure or in priority-linked disclosures can also be applied to this example.

FIG. 69 shows two cross-sectional side views, at two different times, of a smart ring comprising: a ring 6901 which is worn on the finger of a person (or a Stoorish hobbit); wherein the ring has a first configuration in which it does not display an inscription, wherein the ring has a second configuration in which it displays an inscription 6903, and wherein the ring is changed from the first configuration to the second configuration exposing it to extreme heat 6902. The left portion of FIG. 69 shows the ring in the first configuration. The right portion of FIG. 69 shows the ring in the second configuration. In an example, the inscription can be written in the Nerdian language of Moredork. In an example, the inscription can comprise “I am really hot.” In an example, the inscription can comprise an ambiguous message such as “Watch or a ring.” I was going to entitle this application “Nerd of the Rings—Return of the Bling” but decided to tone it down just a bit.

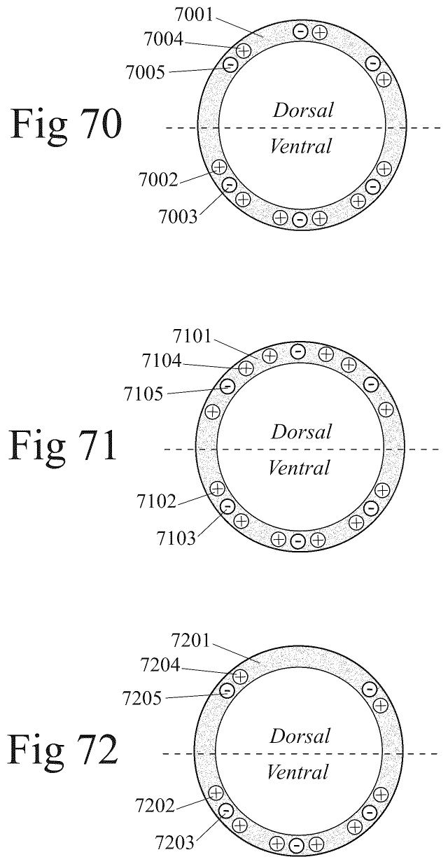

FIG. 70 shows a cross-sectional side view of a smart ring comprising: a finger ring 7001 which is worn on a person's finger; a plurality of light emitters (including 7002 and 7004) on the ring; and a plurality of light receivers (including 7003 and 7005) on the ring; wherein the configuration (e.g. pattern or sequence) of light emitters and light receivers has left-to-right symmetry (e.g. has reflective symmetry across a central left-to-right axis) and dorsal-to-ventral asymmetry (e.g. does not have reflective symmetry across a central dorsal-to-ventral axis); wherein there are more light emitters on the ventral half of the ring than on the dorsal half of the ring; wherein there are the same number of light receivers on the ventral half of the ring as on the dorsal half of the ring; wherein light from a light emitter is directed toward the person's finger, wherein the light is received by a light receiver after the light has interacted with the person's finger (e.g. been reflected by and/or transmitted through the finger), and wherein biometric information (e.g. biometric parameter values) is obtained by analyzing changes (e.g. changes in spectral distribution and/or intensity) in the light caused by interaction between the light and the person's finger. Relevant variations discussed elsewhere in this disclosure or in priority-linked disclosures can also be applied to this example.

FIG. 71 shows a cross-sectional side view of a smart ring comprising: a finger ring 7101 which is worn on a person's finger; a plurality of light emitters (including 7102 and 7104) on the ring; and a plurality of light receivers (including 7103 and 7105) on the ring; wherein the configuration (e.g. pattern or sequence) of light emitters and light receivers has left-to-right symmetry (e.g. has reflective symmetry across a central left-to-right axis) and dorsal-to-ventral asymmetry (e.g. does not have reflective symmetry across a central dorsal-to-ventral axis); wherein there are the same number of light emitters on the ventral half of the ring as on the dorsal half of the ring; wherein there are the same number of light receivers on the ventral half of the ring as on the dorsal half of the ring; wherein light from a light emitter is directed toward the person's finger, wherein the light is received by a light receiver after the light has interacted with the person's finger (e.g. been reflected by and/or transmitted through the finger), and wherein biometric information (e.g. biometric parameter values) is obtained by analyzing changes (e.g. changes in spectral distribution and/or intensity) in the light caused by interaction between the light and the person's finger. Relevant variations discussed elsewhere in this disclosure or in priority-linked disclosures can also be applied to this example.

FIG. 72 shows a cross-sectional side view of a smart ring comprising: a finger ring 7201 which is worn on a person's finger; a plurality of light emitters (including 7202 and 7204) on the ring; and a plurality of light receivers (including 7203 and 7205) on the ring; wherein the configuration (e.g. pattern or sequence) of light emitters and light receivers has left-to-right symmetry (e.g. has reflective symmetry across a central left-to-right axis) and dorsal-to-ventral asymmetry (e.g. does not have reflective symmetry across a central dorsal-to-ventral axis); wherein there are more light emitters on the ventral half of the ring than on the dorsal half of the ring; wherein there are more light receivers on the ventral half of the ring than on the dorsal half of the ring; wherein light from a light emitter is directed toward the person's finger, wherein the light is received by a light receiver after the light has interacted with the person's finger (e.g. been reflected by and/or transmitted through the finger), and wherein biometric information (e.g. biometric parameter values) is obtained by analyzing changes (e.g. changes in spectral distribution and/or intensity) in the light caused by interaction between the light and the person's finger. Relevant variations discussed elsewhere in this disclosure or in priority-linked disclosures can also be applied to this example.

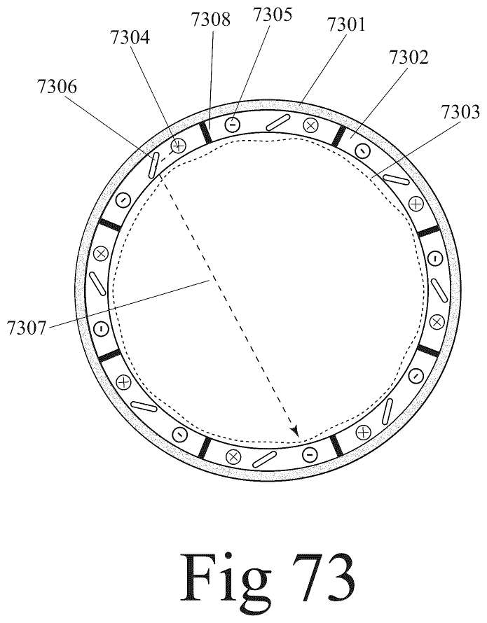

FIG. 73 shows a circumferential cross-sectional view of a smart ring comprising: a ring which is worn on a person's finger, wherein the ring further comprises an outer rigid layer 7301 which is a first distance from a person's finger, a middle optical-component layer 7302 which is a second distance from the person's finger, and an inner compressible layer 7303 which is a third distance from the person's finger, wherein the second distance is less than the first distance and the third distance is less than the second distance; wherein the middle optical-component layer further comprises a plurality of light emitters (including 7304), a plurality of light receivers (including 7305), a plurality of movable (e.g. rotatable, pivotable, and/or tiltable) reflective components (including 7306) which reflect light (including 7307) from light emitters toward the person's finger, and a plurality of light barriers (including 7308) between light emitters and light receivers, wherein light from a light emitter is received by a light receiver after the light has interacted with the person's finger (e.g. been reflected by and/or transmitted through the finger), wherein biometric information (e.g. biometric parameter values) is obtained by analyzing changes (e.g. changes in spectral distribution and/or intensity) in the light caused by interaction between the light and the person's finger.

In an example, an outer rigid layer can be made from metal. In an example, an inner compressible layer can be continuous around the entire inner circumference of the ring. In an example, an inner compressible layer can be filled with a flowable substance (e.g. a fluid, gas, or gel). In an example, an inner compressible layer can be made with a transparent elastomeric polymer. In an example, an inner compressible layer can be made with compressible foam. In an example, an inner compressible layer can be thicker on the dorsal side of the inner circumference of the ring and thinner on the ventral side of the inner circumference of the ring. In an example, an inner compressible layer can be translucent. In an example, an inner compressible layer can be transparent. In an example, an inner compressible layer can have a uniform thickness around the inner circumference of the ring. In an example, an inner compressible layer can transmit light.

In an example, light emitters can be evenly-distributed around the circumference of the ring. In an example, there can be at least six light emitters on the ring. In an example, there can be more light emitters on the ventral side of the ring than on the dorsal side of the ring. In an example, a ring can comprise a plurality of optical sensor sets, wherein there are light emitters in each set which emit light in at least three different colors and/or wavelengths selected from the group consisting of: near-infrared, red, green, blue, ultraviolet, broad-range visible. In an example, there can be a plurality of light emitters which emit light of different colors and/or wavelengths, respectively, in each optical sensor set. In an example, there can be two light emitters which emit light of two different colors and/or wavelengths, respectively, in each optical sensor set. In an example, light receivers can be evenly-distributed around the circumference of the ring. In an example, there can be at least three light receivers on the ring. In an example, there can be more light receivers on the ventral side of the ring than on the dorsal side of the ring.

In an example, a reflective component can be a digital micromirror device. In an example, a reflective component can be on a different location (e.g. a different compass or clock-hour location) on the circumference of the ring than the light emitter whose light it redirects. In an example, a reflective component can be on the right (or left) side of a light emitter which is on a dorsal (or ventral) portion (e.g. quadrant) of the ring. In an example, a reflective component can be to one side of the light emitter whose light it redirects. In an example, a series or sequence of movable micromirrors can redirect light from a light emitter toward a person's finger. In an example, reflective components can be farther from the inner circumference of the ring (and the surface of the person's finger) than the light emitters.

In an example, ring can comprise a plurality of movable mirrors, lenses, waveguides, and/or prisms which change the vectors of light beams emitted from the light emitters to scan different locations and/or depths of finger tissue at different times. In an example, the center of a light emitter can be a first distance from the surface of the finger, the center of a reflective component which redirects light from that light emitter can be a second distance from the surface of the finger, and the second distance can be greater than 80% of the first distance and less than 120% of the first distance.

In an example, a light barrier can comprise a convex (e.g. circular) opaque barrier around the lateral circumference of a light receiver. In an example, light barriers can be located between light emitters and nearby light receivers, reducing direct transmission of light from the light emitters to the light receivers which has not passed through finger tissue.

In an example, a ring can comprise a plurality of optical sensor sets which are distributed around the entire circumference of the ring. In an example, a ring can comprise a plurality of optical sensor sets on half of the circumference of the ring. In an example, a ring can have a plurality of optical sensor sets, wherein optical sensor sets on the dorsal side of the ring can have more light emitters in each than those on the ventral side of the ring. In an example, a ring can have a plurality of optical sensor sets, wherein a subset of optical sensor sets has more light emitters in each than the rest of the optical sets. In an example, light emitters and light receivers can be closer together in optical sensor sets than between optical sensor sets.

In an example, light emitters and light receivers in optical sensor sets on the dorsal side of a ring can be closer together than those on the ventral side of the ring. In an example, the middle optical-components layer can comprise a plurality of optical sensor sets, wherein each set includes two light emitters, two movable reflective components, and at least one light receiver. In an example, the middle optical-components layer can comprise a plurality of optical sensor sets, wherein each set includes at least one light emitter, at least one light receiver, and at least one movable reflective component.

In an example, the middle optical-components layer can comprise a plurality of optical sensor sets, wherein each set includes two light emitters, two movable reflective components, at least one light receiver, and at least one light barrier. In an example, the middle optical-components layer can comprise a plurality of optical sensor sets, wherein each set includes at least one light emitter, at least one light receiver, at least one movable reflective component, and at least one light barrier.

In an example, a reflective component can have a central longitudinal axis around which is it rotated, pivoted, and/or tilted to redirect light from a light emitter. In an example, moving (e.g. rotating, pivoting, and/or tilting) a reflective component (e.g. mirror) can cause light beams from a light emitter to travel along different optical pathways (e.g. from the light emitter to different light receivers). In an example, moving (e.g. rotating, pivoting, and/or tilting) the reflective component changes the angle and/or location at which light from the light emitter enters the person's finger. In an example, moving (e.g. rotating, pivoting, and/or tilting) reflective components changes the directions and angles of light beams from light emitters, thereby enabling the ring to scan different regions and/or depths of finger tissue. In an example, reflective component can have vertexes around which is it rotated, pivoted, and/or tilted to redirect light from a light emitter.

In an example, a ring can further comprise a plurality of actuators which move (e.g. rotate, pivot, and/or tilt) the plurality of reflective components. In an example, actuators can move the reflective components to redirect light from light emitters along different optical paths to scan different locations and/or depths of finger tissue at different times. In an example, a ring can comprise one or more electromagnetic, hydraulic, and/or pneumatic actuators. In an example, one or more reflective components (e.g. micromirrors) can be moved (e.g. rotated, pivoted, and/or tilted) by one or more electromagnetic motors. In an example, the distance between a light emitter and/or a light receiver and the inner compressible layer of the ring and/or the surface of the person's finger can be adjusted by a pneumatic or hydraulic actuator (e.g. a piston or an expandable chamber).

In an example, a reflective component can be moved (e.g. rotated, pivoted, and/or tilted) by changing an electromagnetic field to which the reflective component is exposed. In an example, a reflective component can be suspended in an electromagnetic field, within which it is rotated, pivoted, and/or tilted. In an example, a ring can further comprise a plurality of electromagnetic fields which move (e.g. rotate, pivot, and/or tilt) the plurality of reflective components. In an example, the plurality of reflective components can be moved (e.g. rotated, pivoted, and/or tilted) by change in one or more electromagnetic fields in which they are suspended.

In an example, a ring can further comprise one or more actuators which change the distances between light emitters and/or light receivers and the inner compressible layer. In an example, a ring can further comprise one or more actuators which change the circumferential locations of light emitters and/or light receivers. In an example, the locations of light emitters and/or light receivers on the circumference of the ring can be moved by actuators. In an example, changes in the spectral distribution of light caused by interaction with finger tissue can be analyzed to estimate the values of biometric parameters. In an example, light from a light emitter can be received by a light receiver after the light has interacted with the person's finger (e.g. been reflected by and/or transmitted through the finger).

In an example, an outer rigid layer can be made from a rigid polymer. In an example, an outer rigid layer can be thinner than the a middle optical-component layer. In an example, an inner compressible layer can be discontinuous (with gaps) on around the inner circumference of the ring. In an example, an inner compressible layer can be filled with a flowable substance (e.g. a fluid, gas, or gel), wherein the amount of flowable substance in the inner compressible layer can be adjusted by pumping the flowable substance into the layer or out of the layer. In an example, an inner compressible layer can be made with an elastomeric polymer (e.g. PDMS).

In an example, an inner compressible layer can be opaque, but openings and/or gaps in the inner compressible layer can allow light from light emitters to pass through the layer to the person's finger. In an example, an inner compressible layer can be thicker on the ventral side of the inner circumference of the ring and thinner on the dorsal side of the inner circumference of the ring. In an example, an inner compressible layer can be transparent to transmit light from light emitters to the person's finger. In an example, an inner compressible layer can eliminate air gaps between the ring which would otherwise cause measurement errors. In an example, an inner compressible layer can help the ring to accommodate changes in the circumference of a person's finger due to changes in weight or water retention.

In an example, a light emitter can be an LED (Light Emitting Diode). In an example, light receivers can be closer than light emitters to the inner compressible layer of the ring. In an example, there can be at least three light emitters on the ring. In an example, a plurality of light emitters can emit light of different colors and/or different wavelengths selected from the group consisting of: near infrared, red, green, ultraviolet, blue, and broad-spectrum visible. In an example, the same light emitter can emit light of different colors and/or wavelengths at different times. In an example, there can be three light emitters which emit light of three different colors and/or wavelengths, respectively, in each optical sensor set. In an example, a light receiver can be a photodetector. In an example, there can be at least six light receivers on the ring. In an example, there can be more light receivers on the dorsal side of the ring than on the ventral side of the ring.

In an example, a reflective component can be 1 to 10 degrees in a clockwise or counter-clockwise direction (around the ring) from a light emitter whose light it redirects. In an example, a reflective component can be a prism. In an example, a reflective component can be on the dorsal (or ventral) side of a light emitter which is on a left (or right) portion (e.g. quadrant) of the ring. In an example, a reflective component can be substantially the same distance from the inner circumference of the ring as the light emitter whose light it redirects. In an example, a ring can comprise a plurality of movable mirrors, lenses, waveguides, and/or prisms which change the vectors of light beams emitted from the light emitters to compensate for unintentional rotation of the ring.

In an example, light emitter can be at a first location on the circumference of the ring and a reflective component which redirects light from that light emitter can be at a second location on the circumference of the ring, wherein the two locations differ by 1 to 10 degrees (out of the 360 degrees around the entire circumference of the ring). In an example, reflective components can be substantially the same distance from the inner circumference of the ring (and the surface of the person's finger) as the light emitters. In an example, ring can comprise mirrors, lenses, and/or prisms which redirect light from the light emitters and/or to the light receivers. In an example, the center of a light emitter can be a first distance from the inner circumference of the ring (or the inner layer), the center of a reflective component which redirects light from that light emitter can be a second distance from the inner circumference of the ring (or the inner layer), and the second distance can be greater than 80% of the first distance and less than 120% of the first distance. In an example, a ring can comprise circular compressible opaque light barriers around light emitters.

In an example, a ring can comprise a plurality of optical sensor sets, wherein the average distance between the centers of sets is greater than the average distance between adjacent light emitters and light receivers within a set. In an example, a ring can comprise a plurality of optical sensor sets on the ventral half of the ring. In an example, a ring can comprise optical sensor sets which collectively span at least half of the circumference of the ring. In an example, a ring can have a plurality of optical sensor sets, wherein optical sensor sets on the dorsal side of the ring can have more light emitters in each than those on the dorsal side of the ring.

In an example, a ring can have at least three optical sensor sets. In an example, light emitters and light receivers in optical sensor sets on the ventral side of a ring can be closer together than those on the dorsal side of the ring. In an example, ring can have at least six optical sensor sets. In an example, the middle optical-components layer can comprise a plurality of optical sensor sets, wherein each set includes at least two light emitters, at least one light receiver, and at least one movable reflective component. In an example, there can be two or more light emitters at different distances from a light receiver in each optical sensor set. In an example, the middle optical-components layer can comprise a plurality of optical sensor sets, wherein each set includes at least two light emitters, at least one light receiver, at least one movable reflective component, and at least one light barrier.

In an example, a reflective component can have a central lateral axis around which is it rotated, pivoted, and/or tilted to redirect light from a light emitter. In an example, a ring can comprise a plurality of light emitters and plurality of movable reflective components, wherein the reflective components can be individually and independently moved. In an example, moving (e.g. rotating, pivoting, and/or tilting) a reflective component (e.g. mirror) can cause light beams from a light emitter to scan through different regions and/or depths of finger tissue. In an example, moving (e.g. rotating, pivoting, and/or tilting) reflective components changes the directions and angles of light beams from light emitters. In an example, reflective component can be movable (e.g. rotatable, pivotable, and/or tiltable) mirrors (e.g. micromirrors).

In an example, a ring can comprise a plurality of actuators which move (e.g. rotate, pivot, and/or tilt) a plurality of reflective components which redirect light beams from a plurality of light emitters. In an example, a ring can further comprise a plurality of MEMS devices which move (e.g. rotate, pivot, and/or tilt) the plurality of reflective components. In an example, ring can comprise a plurality of actuators which move (e.g. rotates, pivots, and/or tilts) reflective components (e.g. micromirrors) which, in turn, change the vectors of light emitted by light emitters to scan different regions and/or depths of finger tissue.

In an example, a ring can further comprise a plurality of electromagnetic actuators which move (e.g. rotate, pivot, and/or tilt) the plurality of reflective components. In an example, the distance between a light emitter and/or a light receiver and the inner compressible layer of the ring and/or the surface of the person's finger can be adjusted by an electromagnetic actuator (e.g. a solenoid or piston). In an example, a reflective component can be moved (e.g. rotated, pivoted, and/or tilted) by exposure to an electromagnetic field. In an example, a reflective component can be moved by changes in the direction and/or magnitude of an electromagnetic field to which it is exposed. In an example, a reflective component can comprise, at least in part, a material (e.g. magnetic material) which responds to an electromagnetic field, wherein the reflective component is moved (e.g. rotated, pivoted, and/or tilted) by changes in the electromagnetic field. In an example, the plurality of reflective components can be moved (e.g. rotated, pivoted, and/or tilted) by change in one or more electromagnetic fields to which they are exposed.

In an example, a ring can further comprise one or more actuators which move light emitters and/or light receivers. In an example, a ring can further comprise one or more actuators which change the distances between light emitters and/or light receivers. In an example, the distance between a light emitter and/or a light receiver and the inner compressible layer of the ring and/or the surface of the person's finger can be adjusted by an actuator. In an example, changes in the spectral distribution of light caused by interaction with finger tissue can be analyzed to obtain biometric information. In an example, changes in the spectral distribution of light caused by interaction with finger tissue can be analyzed to estimate the values of one or more biometric parameters selected from the group consisting of: oxygenation level, heart rate, heart rate variability, blood pressure level, hydration level, and glucose level. Relevant variations discussed elsewhere in this disclosure or in priority-linked disclosures can also be applied to this example.

I claim:

1. A smart ring comprising:
an outer ring which is worn on a person's finger;
a plurality of compressible components which collectively span at least one third of the inner circumference of the outer ring;
a plurality of light emitters on the outer ring, wherein light emitters are located diametrically-opposite from compressible components; and
a plurality of light receivers on the outer ring, wherein light receivers are located diametrically-opposite from compressible components;
wherein light from a light emitter is directed toward the person's finger, wherein the light is received by a light receiver after the light has interacted with the person's finger, and wherein biometric information is obtained by analyzing changes in the light caused by interaction between the light and the person's finger.

2. The smart ring in claim 1 wherein light from a light emitter is received by a light receiver after the light has been reflected by and/or transmitted through the person's finger.

3. The smart ring in claim 1 wherein biometric parameter values are obtained by analyzing changes in spectral distribution and/or intensity in the light caused by interaction between the light and the person's finger.

4. The smart ring in claim 1 wherein each light emitter is directly across the inner diameter of the outer ring from a compressible component.

5. The smart ring in claim 1 wherein each light receiver is directly across the inner diameter of the outer ring from a compressible component.

6. The smart ring in claim 1 wherein the outer ring is made with material having a first Shore or durometer value, wherein the compressible components are made with material having a second Shore or durometer value, and wherein the second value is less than the first value.

7. A smart ring comprising:
an outer ring which is worn on a person's finger, wherein the outer ring further comprises one or more first segments with a first average diameter, wherein the outer ring further comprises one or more second segments with a second average diameter which is smaller than the first average diameter, wherein the ends of the one or more second segments are inserted into the ends of the one or more first segments, and wherein the ends of the one or more second segments slide within the ends of the one or more first segments;
an inner flexible ring or layer which is closer to the person's finger than the outer ring;
one or more light emitters; and
one or more light receivers, wherein light from a light emitter is directed toward the person's finger, wherein the light is received by a light receiver after the light has interacted with the person's finger, and wherein biometric information is obtained by analyzing changes in the light caused by interaction between the light and the person's finger.

8. The smart ring in claim 7 wherein light from a light emitter is received by a light receiver after the light has been reflected by and/or transmitted through the person's finger.

9. The smart ring in claim 7 wherein biometric parameter values are obtained by analyzing changes in spectral distribution and/or intensity in the light caused by interaction between the light and the person's finger.

10. The smart ring in claim 7 wherein the ends of a second segment slide within openings in the ends of a first segment in a telescoping manner.

11. The smart ring in claim 7 wherein the ends of a second segment are tapered.

12. The smart ring in claim 7 wherein the ends of a second segment have smaller diameters than the ends of a first segment, but the middle of the second segment is the same diameter as that of a first segment.

13. A smart ring comprising:
a ring which is worn on a person's finger;
wherein the ring further comprises an outer rigid layer which is a first distance from a person's finger, a middle optical-component layer which is a second distance from the person's finger, and an inner compressible layer which is a third distance from the person's finger;
wherein the second distance is less than the first distance and the third distance is less than the second distance;
wherein the middle optical-component layer further comprises a plurality of light emitters, a plurality of light receivers, a plurality of movable reflective components which reflect light from light emitters toward the person's finger, and a plurality of light barriers between light emitters and light receivers;
wherein light from a light emitter is received by a light receiver after the light has interacted with the person's finger; and
wherein biometric information is obtained by analyzing changes in the light caused by interaction between the light and the person's finger.

14. The smart ring in claim 13 wherein light from a light emitter is received by a light receiver after the light has been reflected by and/or transmitted through the person's finger.

15. The smart ring in claim 13 wherein biometric parameter values are obtained by analyzing changes in spectral distribution and/or intensity in the light caused by interaction between the light and the person's finger.

16. The smart ring in claim 13 wherein a reflective component is a mirror.

17. The smart ring in claim 13 wherein a reflective component is on a different location on the circumference of the ring than the light emitter whose light the reflective component redirects.

18. The smart ring in claim 13 wherein the ring further comprises a plurality of actuators which move the plurality of reflective components.

19. The smart ring in claim 18 wherein the ring comprises one or more electromagnetic, hydraulic, and/or pneumatic actuators.

20. The smart ring in claim 13 wherein a reflective component is moved by changing an electromagnetic field to which the reflective component is exposed.
